(19) United States
(12) Patent Application Publication
(10) Pub. No.: US 2025/0049032 A1
(43) Pub. Date: Feb. 13, 2025

(54) ETHYLENE SIGNALING ACTIVATOR MODULATES ROOT SYSTEM ARCHITECTURE

(71) Applicant: Salk Institute for Biological Studies, La Jolla, CA (US)

(72) Inventors: Wolfgang Busch, San Diego, CA (US); Wenrong He, La Jolla, CA (US)

(73) Assignee: Salk Institute for Biological Studies, La Jolla, CA (US)

(21) Appl. No.: 18/719,805

(22) PCT Filed: Dec. 16, 2022
(86) PCT No.: PCT/US2022/081873

§ 371 Date: Jun. 13, 2024

Related U.S. Application Data
(60) Provisional application No. 63/291,321, filed on Dec. 17, 2021.

(51) Int. Cl. A01N 43/56; A01P 21/00

(52) U.S. Cl. CPC A01N 43/56; A01P 21/00

(57) ABSTRACT

The present disclosure provides compositions and methods for regulating ethylene signaling in a plant or a plant tissue culture. The present disclosure also provides compositions and methods for modulating gravitropic set-point angle in plant roots via regulation of ethylene signaling. The present disclosure further provides small molecules that regulate ethylene signaling.

### CROSS-REFERENCE TO RELATED APPLICATION

This application claims priority to, and the benefit of U.S. Provisional Patent Application No. 63/291,321, Dec. 17, 2021, which is incorporated by reference herein in its entirety for all purposes.

### DESCRIPTION OF THE TEXT FILE SUBMITTED ELECTRONICALLY

The Sequence Listing XML associated with this application is provided electronically in XML file format and is hereby incorporated by reference into the specification. The name of the XML file containing the Sequence Listing XML is SALK_008_01WO_SeqList_ST26. The XML file is 18,066 bytes, created on Dec. 15, 2022, and is being submitted electronically via USPTO Patent Center.

### FIELD

The present disclosure generally relates to the field of ethylene signaling in plants. More particularly, the present disclosure relates to compositions and methods for modulating gravitropic set-point angle in plant roots via regulation of ethylene signaling.

### BACKGROUND OF THE DISCLOSURE

The lateral root angle or gravitropic set-point angle (GSA) is an important trait for root system architecture (RSA) that determines the radial expansion of the root system. The GSA therefore plays a crucial role in the ability of plants to access nutrients and water in the soil. Despite its importance, only few regulatory pathways and mechanisms that determine GSA are known, and these mostly relate to auxin and cytokinin pathways.

Here we describe the identification of a small molecule that modulates GSA in Arabidopsis thaliana roots and acts via the activation of ethylene signaling. We discovered that this small molecule Mebendazole (MBZ) directly acts on the serine/threonine protein kinase CTR1, which is a negative regulator of ethylene signaling. Our research not only reveals that the ethylene signaling pathway is essential for GSA regulation, but it also identifies a small molecular modulator of RSA and the first specific activator of ethylene signaling.

### SUMMARY OF THE DISCLOSURE

As set forth herein, to identify potential regulators of GSA, we performed chemical genetics screens and identified a small molecule, Mebendazole (MBZ), which dramatically increased GSA. Further genetic analysis revealed that MBZ increased GSA through rapidly activating the ethylene signaling pathway. Both MBZ and 1-aminocy-clopropane-1-carboxylic acid (ACC) treatment induced ethylene signaling, which led to a modulation of lateral root angle. Further molecular and biochemistry studies showed that MBZ activates ethylene signaling by specifically targeting a serine/threonine protein kinase, Constitutive Triple Response 1 (CTR1) and thereby blocking its kinase activity. As set forth herein, this not only exposes MBZ as an efficient small molecular activator of ethylene signaling, but also that ethylene signaling plays an important role in regulating gravitropic set-point angle (GSA).

The compositions and methods of the present disclosure provide plants with modified, shallower root architectures that impart benefits such as making them more useful for phosphorous recovery and for improving their ability to more effectively utilize rain water and surface moisture in dryer regions where water might evaporate faster on or near the surface.

In some embodiments as provided herein are compositions, combinations, processes, systems, and kits comprising a small molecule and their use for changing the root system architecture of a plant or plant tissue culture.

In some embodiments as provided herein are compositions, combinations, processes, systems, and kits comprising a small molecule and their use for changing the lateral root angle of a plant or plant tissue culture.

In some embodiments as provided herein are compositions, combinations, processes, systems, and kits comprising a small molecule and their use for changing the gravitropic set-point angle of a plant or plant tissue culture.

In some embodiments as provided herein are compositions, combinations, processes, systems, and kits comprising a small molecule and their use for regulating ethylene signaling in a plant or plant tissue culture.

In some embodiments as provided herein are methods of modulating an ethylene signaling pathway in a plant or plant tissue culture comprising administering a small molecule that acts as a regulator of ethylene signaling.

In some embodiments of the present disclosure, the small molecules have a molecular weight less than 300 g/mol.

In some embodiments of the present disclosure, the small molecules inhibit or block the kinase activity of CTR1.

In some embodiments of the present disclosure, the small molecules are positive regulators of ethylene signaling.

In some embodiments of the present disclosure, the small molecules inhibit a negative regulator of ethylene signaling.

In some embodiments of the present disclosure, the small molecules modulate the lateral root angle (GSA) of the plant or plant tissue culture.

In some embodiments of the present disclosure, the small molecules modulate the gravitropic set-point angle (GSA) of the plant or plant tissue culture.

In some embodiments of the present disclosure, GSA is significantly increased. In some embodiments of the present disclosure, the resultant modulating leads to changes in root system architecture of the plant or plant tissue culture relative to a check plant or plant tissue culture, respectively, that is not administered the small molecule.

In some embodiments of the present disclosure, the resultant modulating leads to changes in lateral root angle of the plant or plant tissue culture when compared to a check plant or plant tissue culture, respectively, that is not administered the small molecule.

In some embodiments of the present disclosure, the resultant changes in lateral root angle cause the lateral roots of the plant or plant tissue culture to grow in a more horizontal direction when compared to a check plant or plant tissue culture, respectively, that is not administered the small molecule.

In some embodiments of the present disclosure, the administering is accomplished by adding the small molecule to a growing medium used to grow the plant or the plant tissue culture.

In some embodiments of the present disclosure, the administering is accomplished by applying the small molecule to the growing medium used to grow the plant or the plant tissue culture.

In some embodiments of the present disclosure, the applying is accomplished by spraying the small molecule onto the plant or plant tissue culture.

In some embodiments of the present disclosure, the applying is accomplished by using a liquid comprising the small molecule.

In some embodiments of the present disclosure, the small molecule that is used is an anthelmintic agent.

In some embodiments of the present disclosure, the small molecule that is used is a synthetic benzimidazole derivate.

In some embodiments of the present disclosure, the small molecule that is used is a benzimidazole anthelmintic agent. In some embodiments of the present disclosure, the small molecule that is used is Mebendazole.

In some embodiments as provided herein are methods of activating an ethylene signaling pathway in a plant or a plant tissue culture comprising the steps of administering to a plant or a plant tissue culture a small molecule that targets CTR1 protein that is a negative modulator of the ethylene signalling pathway.

In some embodiments of the present disclosure, said small molecule inhibits a kinase activity of CTR1.

In some embodiments of the present disclosure, said small molecule binds to a pocket of the CTR1 kinase domain.

In some embodiments of the present disclosure, the CTR1 kinase domain is present in SEQ ID NO: 3.

In some embodiments of the present disclosure, the small molecule is mebendazole.

Additional embodiments of the present disclosure will be readily ascertained by one skilled in the art of molecular genetics, plant breeding, plant husbandry, agricultural production, and other plant-related technologies upon reading the present application.

### BRIEF DESCRIPTION OF THE DRAWINGS

FIGS. 1A-1F show that MBZ treatment perturbs root system architecture (RSA). FIG. 1A, 14-day-old seedlings of Arabidopsis grown on DMSO and MBZ (1 μM) plates. FIG. 1B, Quantification of gravitropic setpoint angle (GSA) in FIG. 1A. 15 lateral roots (LRs) per seedling on DMSO plates, 12 LRs per seedling on MBZ plates, and 6 seedlings for each condition were used. Unpaired, two-tailed Student's t-tests was used for statistical analysis. **** p<0.0001. FIG. 1C, 5-day-old seedlings on DMSO and MBZ (1 μM) plates. FIG. 1D, Quantification of primary root length (cm) in FIG. 1C. 15-20 seedlings for each treatment were used. **** p<0.0001. FIG. 1E, 12-day-old (upper panel) Arabidopsis seedlings on DMSO plates were transferred to MBZ (1 μM) plates, and 17-day-old (bottom panel) seedlings on MBZ (1 μM) plates were transferred to DMSO plates, followed by continued scanning for 24 h. FIG. 1F, Changes of lateral root angle after plate transfer from DMSO to MBZ (lines above timepoint 0; positive angle changes; MBZ1, MBZ2, MBZ3, MBZ4, and MBZ5) or MBZ to DMSO (lines belove timepoint 0; negative angle changes; DMSO1, DMS02, DMS03, DMSO104, and DMS05) compared to timepoint 0. (Scale bar: FIG. 1A, 1C, 1G, 1 cm; FIG. 1E, 5 mm.). Boxplots: Whiskers: min/max values; hinges: 25^{th} to 75^{th} percentile; mid-line: median.

FIGS. 2A-2E show that MBZ treatment regulates the ethylene pathway. FIG. 2A, Gene ontology (GO) analysis upregulated genes (log 2 fold change >1) upon MBZ treatment. FIG. 2B, Heatmap of genes involved in the ethylene pathway, which were regulated by MBZ treatment. FIGS. 2C-2E, Venn diagrams showing differentially expressed genes (abs(log 2) fold change >1) in 4 h MBZ treatment and 4 h ethylene treatment. All genes (FIG. 2C), upregulated genes (FIG. 2D), downregulated genes (FIG. 2E).

FIGS. 3A-3H show MBZ induces ethylene responses and mimics ACC treatment. FIG. 3A, Quantification of Ethylene Response Factor1 (ERF1) expression upon MS, 50 μM ACC, DMSO, and 10 μM MBZ treatments for 2 hours in the Col-0 wildtype. Unpaired, two-tailed Student's t-tests. * p<0.05. Bars represent mean values±s.d. from two technical replicates. Similar results were obtained from three biological replicates of the experiment. FIG. 3B, GFP fluorescence in root tips of ein3 eil1 35S:EIN3-GFP treated for 2 hours with DMSO, 10 μM MBZ, 50 μM ACC. 6-10 seedlings were observed for each treatment. FIG. 3C, 4d etiolated seedlings grown on MS, 10 μMACC, DMSO, 10 μM MBZ plates. 20 seedlings were observed for each treatment. FIGS. 3D-3E, Quantification of hypocotyl length (FIG. 3D) and root length (FIG. 3E) of seedlings in (FIG. 3C). 20 seedlings were observed for each treatment. **** p<0.0001. FIG. 3F, Confocal microscopy images of median longitudinal optical sections of root meristem and mature zone stained with propidium iodide (PI) of 4d etiolated WT seedlings on DMSO, 2.5 μM MBZ, and 10 μM ACC plates. 6-10 seedlings were observed for each treatment. FIGS. 3G-3H, Meristem length (FIG. 3G) and mature cell size (FIG. 3H) of seedlings in (FIG. 3F). 6-10 seedlings for each treatment were used. Different letters label significant different values (p<0.0001). One-way ANOVA and post hoc Tukey testing were used for statistical analysis in FIG. 3G and FIG. 3H. (Scale bar: FIG. 3B, 500 μm; FIG. 3C, 5 mm; FIG. 3F, 100 μm.). Boxplots: Whiskers: min/max values; hinges: 25^{th} to 75^{th} percentile; mid-line: median.

FIGS. 4A-4E show MBZ acts downstream of ethylene biosynthesis. FIG. 4A, 6-day-old seedlings of Col-0, etr1-1, and etr1-3 grown on DMSO and 1.2 μM MBZ plates. 20 seedlings were observed. FIG. 4B, Quantification of root length in FIG. 4A. 20 seedlings were countified. FIG. 4C, 16-day-old seedlings of Col-0 and etr1-1 grown on DMSO and 1.2 μM MBZ plates. 20 seedlings were observed. FIG. 4D, Quantification of GSA in FIG. 4C. 20 seedlings were countified. FIG. 4E, Quantification of ethylene production by GC-MS of 4d etiolated seedlings of Col-0 in vials with DMSO, 10 μM MBZ, and 10 μMACC medium. 100 seedlings for every vial. 4 biological replicates for each treatment, ND means not detectable. One-way ANOVA and post hoc Tukey testing were used for statistical analysis in FIG. 4B and FIG. 4D. Different letters label significant different values (p<0.0001). (Scale bar: FIG. 4A, 4C, 1 cm.). Boxplots: Whiskers: min/max values; hinges: 25^{th} to 75^{th} percentile; mid-line: median.

FIGS. 5A-5G show MBZ targets the ethylene signaling pathway. FIG. 5A, Diagram of ethylene signaling pathway. FIG. 5B, 7-day-old seedlings of Col-0, ctr1-1, and ein2-5 grown on DMSO and 1.2 μM MBZ plates. FIG. 5C, Quantification of primary root length of seedlings in FIG. 5B. 15-20 seedlings were quantified. Similar results were obtained from three biological replicates of the experiment. FIG. 5D, 19-day-old seedlings of Col-0 and ctr1-1 grown on DMSO and 1.3 μM MBZ plates. FIG. 5E, Quantification of GSA of seedlings in FIG. 5D. 10 seedlings were quantified. Similar results were obtained from three biological replicates of the experiment. FIG. 5F, 16 day-old seedlings of Col-0 and ein2-5, ein3eil1 grown on DMSO and 1.3 μM MBZ plates. FIG. 5G, Quantification of GSA of seedlings in FIG. 5F. One-way ANOVA and post hoc Tukey testing were used for statistical analysis in FIGS. 5C, 5E, and 5G. Different letters label significant different values (p<0.0001). (Scale bar: FIG. 5B, 5D, 5F, 1 cm.). Boxplots: Whiskers: min/max values; hinges: 25^{th} to 75^{th} percentile; mid-line: median.

FIGS. 6A-6D show MBZ inhibits CTR1 kinase activity. FIG. 6A, Sequence alignment of MAPK14 in human and CTR1-KD in Arabidopsis around 4 core amino acids (Highlighted by frames) for MBZ binding. The aligned sequences corresponding to kinase domain are present in SEQ ID NO: 7 (hsMAPK14) and SEQ ID NO: 3 (CTR1), respectively. FIG. 6B, Molecular modeling of the interaction between MBZ (small molecule) and CTR1 kinase domain (CTR1-KD). In the upper panel, the key residues that contribute to the binding with CTR1-KD were highlighted. The bottom panel showed the surface (gray) of the catalytic pocket of CTR1-KD. FIG. 6C, Western blot using antibody of RabMAb for CTR1-KD and MAPK4 kinase activity under different concentration of MBZ treatment. Left panel: CTR1-KD; Right panel: MAPK4. MBP is the substrate in both panels, MBZ concentration is 0, 10, and 100 μM as labeled in different reactions, [ATPyS]CTR1-KD: ATPyS binding CTR1-KD, [ATPyS]MAPK4: ATPyS binding MAPK4, kinase activity of CTR1-KD or MAPK4 was represented by MBP that obtained ATPyS from CTR1-KD or MAPK4, which is labeled as [ATPyS]MBP. FIG. 6D, Coomassie blue staining of the gel shown in (c). CTR1: ˜200 ng, MAPK4: ˜1 μg. MBP: 2 μg, ATPyS: 1 mM. MBZ: 0, 10, or 100 μM. Arrows label the position of these bands on the gel.

FIGS. 7A-7B show working model for MBZ action on ethylene signaling. FIG. 7A, ethylene suppresses CTR1 activity to promote EIN2C translocation and activates ethylene signaling. FIG. 7B, MBZ inhibits CTR1 activity by binding to its kinase domain, and promotes EIN2C translocation to activate the ethylene signaling.

FIGS. 8A-8B show MBZ treatment affects root growth and development FIG. 8A, The chemical structure of MBZ. FIG. 8B, The root hair phenotype of 7-day-old seedlings on DMSO and 1 μM MBZ plates recorded by a scanner (left panel) and a microscope (right panel). (Scale bar: FIG. 8B left panel, 13 mm; FIG. 8B right panel, 1000 μm)

FIGS. 9A-9I show MBZ treatment does not directly regulate auxin or cytokinin pathways. FIG. 9A, 7-day-old Col-0 seedlings grown on ½ MS, 20 nM IAA, DMSO, and 1 μM MBZ plates. FIG. 9B, Quantification of primary root length of seedlings in FIG. 9A. FIG. 9C, GFP fluorescence in root tips of 7-day-old seedlings of DR5-GFP V2 grown on DMSO and 1 μM MBZ plates. 10 seedlings were observed. FIG. 9D, 14-day-old Col-0 seedlings on 12 MS, 20 nM IAA, DMSO, and 1 μM MBZ plates. FIG. 9E, Quantification of GSA of seedlings in (FIG. 9D). 20 seedlings were quantified. FIG. 9F, GFP fluorescence in root tips of 7-day-old seedlings of DR5-GFP V2 upon 3 h treatment with H₂O, 10 μM MBZ, and 1 μM IAA. 6-10 seedlings were observed. FIG. 9G, GFP fluorescence in cells of root tips of 6-day-old seedlings of TUB6-GFP upon 1 h treatment with H₂O, 50 μM MBZ, and 1 μM IAA. 6-10 seedlings were observed. FIG. 911, Quantification of auxin marker genes expression upon 2 h treatment with DMSO, and 10 μM MBZ. FIG. 9I, Quantification of ERF1 and ARR5 expression upon DMSO, and 10 μM MBZ treatment for 3 h, 6 h, and 12 h. One-way ANOVA and post hoc Tukey testing were used for statistical analysis in FIG. 9B and FIG. 9D. Different letters label significant different values (p<0.0001); statistical analysis in (FIG. 9H) and (FIG. 9I) were done using unpaired, two-tailed Student's t-tests. * p<0.05, ** p<0.01, *** p<0.001. The chart represents the mean values±s.d. with two technical replicates. Similar results were obtained from three biological replicates of the experiment. P-values are indicated in the figure. (Scale bar: FIG. 9A, 9D, 1 cm; FIG. 9C, 9F, 500 μm). Whiskers: min/max values; hinges: 25^{th} to 75^{th} percentile; mid-line: median.

FIGS. 10A-10F show etiolated seedling phenotypes in mutants of the ethylene pathway. FIG. 10A, 4-day-old etiolated seedlings of Col-0 and etr1-3 grown on DMSO, 2.5 μM MBZ, and 10 μM ACC plates. FIG. 10B-FIG. 10C, Quantification of hypocotyl length (FIG. 10B) and root length (FIG. 10C) of seedlings in (FIG. 10A). 20 seedlings were quantified. FIG. 10D, 4-day-old etiolated seedlings of Col-0, ctr1-1, ein2-5, and ein3eil1 grown on DMSO, 2.5 μM MBZ, and 10 μM ACC plates. FIG. 10E-FIG. 10F, Quantification of hypocotyl length (FIG. 10E) and root length (FIG. 10F) of seedlings in FIG. 10D. 20 seedlings were quantified. Statistical analysis in FIG. 10B, FIG. 10C and FIG. 10E, FIG. 10F were done using unpaired, two-tailed Student's t-tests. P-values are indicated in the figure. (Scale bar: FIG. 10A, FIG. 10D, 1 cm). Whiskers: min/max values; hinges: 25^{th} to 75^{th} percentile; mid-line: median.

FIGS. 11A-11D show alignment of CTR1-KD in Arabidopsis and MAPK14 in human. FIG. 11A, The structure alignment of MAPK14 and CTR1-KD. FIG. 11B, The protein binding sites in MAPK14 and CTR1-KD with highlighted key residues. FIG. 11C, Sequence alignment of kinase domains, which are found within SEQ ID NO: 7 (hsMAPK14), SEQ ID NO: 8 (AtMAPK4), SEQ ID NO: 9 (AtMAPK6), SEQ ID NO: 10 (AtMAPK9), SEQ ID NO: 11 (AtMAPK11), SEQ ID NO: 12 (AtMAPK12), respectively. Key residues in MAPK14_human and CTR1-KD in Arabidopsis are indicated by red boxes. FIG. 11D, Phylogenetic analysis of kinases aligned in FIG. 11C.

FIGS. 12A-12C show high concentrations or long-term treatment of MBZ induce phenotypes that go beyond ethylene induced effects. FIG. 12A, Confocal microscopy images of a root meristem of 4-day-old etiolated WT seedling grown on 10 μM MBZ plate. FIG. 12B, 38-day-old Col-0 plants grown on DMSO and 1.2 μM MBZ plates. FIG. 12C, 14-day-old seedlings of Col-0 grown on increasing concentrations MBZ and ACC (0.1, 0.2, 0.4, 1, 2, 4, 10 μM). 20 seedlings were observed. Similar results were obtained from three biological replicates of the experiment. (Scale bar: FIG. 12A, 100 μm, FIG. 12B and FIG. 12C, 1 cm).

FIG. 13 provides a list of blast hits in Arabidopsis using the MAPK14_human sequence as BLASTP

### DETAILED DESCRIPTION OF THE DISCLOSURE

### I. Definitions

Unless stated otherwise, all technical and scientific terms used herein have the same meaning as commonly understood by those of ordinary skill in the art to which the disclosure belongs. While the following terms are believed to be well understood by one of ordinary skill in the art, the following definitions are set forth to facilitate explanation of the presently disclosed subject matter. Although any methods and materials similar or equivalent to those described herein can be used in the practice or testing of the present disclosure, preferred methods and materials are described. The following terms are defined below. These definitions are for illustrative purposes and are not intended to limit the common meaning in the art of the defined terms.

The term “a” or “an” refers to one or more of that entity, i.e., can refer to a plural referent. As such, the terms “a” or “an”, “one or more” and “at least one” are used interchangeably herein. In addition, reference to “an element” by the indefinite article “a” or “an” does not exclude the possibility that more than one of the elements is present, unless the context clearly requires that there is one and only one of the elements.

As used in this specification, the term “and/or” is used in this disclosure to mean either “and” or “or” unless indicated otherwise.

Throughout this specification, unless the context requires otherwise, the words “comprise”, or variations such as “comprises” or “comprising”, will be understood to imply the inclusion of a stated element or integer or group of elements or integers but not the exclusion of any other element or integer or group of elements or integers.

As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used herein, a small molecule is a low molecular weight (<900 daltons) organic compound that may regulate a biological process, with a size on the order of 1 nm.

As used herein, the term “at least a portion” or “fragment” of a nucleic acid or polypeptide means a portion having the minimal size characteristics of such sequences, or any larger fragment of the full length molecule, up to and including the full length molecule. A fragment of a polynucleotide of the disclosure may encode a biologically active portion of a genetic regulatory element. A biologically active portion of a genetic regulatory element can be prepared by isolating a portion of one of the polynucleotides of the disclosure that comprises the genetic regulatory element and assessing activity as described herein. Similarly, a portion of a polypeptide may be 4 amino acids, 5 amino acids, 6 amino acids, 7 amino acids, and so on, going up to the full length polypeptide. The length of the portion to be used will depend on the particular application. A portion of a nucleic acid useful as a hybridization probe may be as short as 12 nucleotides; in some embodiments, it is 20 nucleotides. A portion of a polypeptide useful as an epitope may be as short as 4 amino acids. A portion of a polypeptide that performs the function of the full-length polypeptide would generally be longer than 4 amino acids. In some embodiments, a fragment of a polypeptide or polynucleotide comprises at least 10%, 20%, 30%, 40%, 50%, 60%, 70%, 80%, 90%, 95%, 96%, 97%, 98%, or 99% of the entire length of the reference polypeptide or polynucleotide. In some embodiments, a polypeptide or polynucleotide fragment may contain 5, 10, 15, 20, 25, 30, 35, 40, 45, 50, 60, 70, 80, 90, 100, 200, 300, 400, 500, 600, 700, 800, 900, 1000, 2000 or more nucleotides or amino acids.

As used herein, the term “codon optimization” implies that the codon usage of a DNA or RNA is adapted to that of a cell or organism of interest to improve the transcription rate of said recombinant nucleic acid in the cell or organism of interest. The skilled person is well aware of the fact that a target nucleic acid can be modified at one position due to the codon degeneracy, whereas this modification will still lead to the same amino acid sequence at that position after translation, which is achieved by codon optimization to take into consideration the species-specific codon usage of a target cell or organism.

As used herein, the term “endogenous” or “endogenous gene,” refers to the naturally occurring gene, in the location in which it is naturally found within the host cell genome. “Endogenous gene” is synonymous with “native gene” as used herein. An endogenous gene as described herein can include alleles of naturally occurring genes that have been mutated according to any of the methods of the present disclosure, i.e. an endogenous gene could have been modified at some point by traditional plant breeding methods and/or next generation plant breeding methods.

As used herein, the term “exogenous” refers to a substance coming from some source other than its native source. For example, the terms “exogenous protein,” or “exogenous gene” refer to a protein or gene from a non-native source, and that has been artificially supplied to a biological system. As used herein, the term “exogenous” is used interchangeably with the term “heterologous,” and refers to a substance coming from some source other than its native source.

The terms “genetically engineered host cell,” “recombinant host cell,” and “recombinant strain” are used interchangeably herein and refer to host cells that have been genetically engineered by the methods of the present disclosure. Thus, the terms include a host cell (e.g., bacteria, yeast cell, fungal cell, CHO, human cell, plant cell, protoplast derived from plant, callus, etc.) that has been genetically altered, modified, or engineered, such that it exhibits an altered, modified, or different genotype and/or phenotype (e.g., when the genetic modification affects coding nucleic acid sequences), as compared to the naturally-occurring host cell from which it was derived. It is understood that the terms refer not only to the particular recombinant host cell in question, but also to the progeny or potential progeny of such a host cell.

As used herein, the term “heterologous” refers to a substance coming from some source or location other than its native source or location. In some embodiments, the term “heterologous nucleic acid” refers to a nucleic acid sequence that is not naturally found in the particular organism. For example, the term “heterologous promoter” may refer to a promoter that has been taken from one source organism and utilized in another organism, in which the promoter is not naturally found. However, the term “heterologous promoter” may also refer to a promoter that is from within the same source organism, but has merely been moved to a novel location, in which said promoter is not normally located.

Heterologous gene sequences can be introduced into a target cell by using an “expression vector,” which can be a eukaryotic expression vector, for example a plant expression vector.

Methods used to construct vectors are well known to a person skilled in the art and described in various publications. In particular, techniques for constructing suitable vectors, including a description of the functional components such as promoters, enhancers, termination and polyadenylation signals, selection markers, origins of replication, and splicing signals, are reviewed in the prior art. Vectors may include but are not limited to plasmid vectors, phagemids, cosmids, artificial/mini-chromosomes (e.g. ACE), or viral vectors such as baculovirus, retrovirus, adenovirus, adeno-associated virus, herpes simplex virus, retroviruses, bacteriophages. The eukaryotic expression vectors will typically contain also prokaryotic sequences that facilitate the propagation of the vector in bacteria such as an origin of replication and antibiotic resistance genes for selection in bacteria. A variety of eukaryotic expression vectors, containing a cloning site into which a polynucleotide can be operatively linked, are well known in the art and some are commercially available from companies such as Stratagene, La Jolla, Calif.; Invitrogen, Carlsbad, Calif.; Promega, Madison, Wis. or BD Biosciences Clontech, Palo Alto, Calif. In one embodiment the expression vector comprises at least one nucleic acid sequence which is a regulatory sequence necessary for transcription and translation of nucleotide sequences that encode for a peptide/polypeptide/protein of interest.

As used herein, the term “naturally occurring” as applied to a nucleic acid, a polypeptide, a cell, or an organism, refers to a nucleic acid, polypeptide, cell, or organism that is found in nature. The term “naturally occurring” may refer to a gene or sequence derived from a naturally occurring source. Thus, for the purposes of this disclosure, a “non-naturally occurring” sequence is a sequence that has been synthesized, mutated, engineered, edited, or otherwise modified to have a different sequence from known natural sequences. In some embodiments, the modification may be at the protein level (e.g., amino acid substitutions). In other embodiments, the modification may be at the DNA level (e.g., nucleotide substitutions).

As used herein, the term “nucleotide change” or “nucleotide modification” refers to, e.g., nucleotide substitution, deletion, and/or insertion, as is well understood in the art. For example, such nucleotide changes/modifications include mutations containing alterations that produce silent substitutions, additions, or deletions, but do not alter the properties or activities of the encoded protein or how the proteins are made. As another example, such nucleotide changes/modifications include mutations containing alterations that produce replacement substitutions, additions, or deletions, that alter the properties or activities of the encoded protein or how the proteins are made.

As used herein, the term “protein modification” refers to, e.g., amino acid substitution, amino acid modification, deletion, and/or insertion, as is well understood in the art.

The term “next generation plant breeding” refers to a host of plant breeding tools and methodologies that are available to today's breeder. A key distinguishing feature of next generation plant breeding is that the breeder is no longer confined to relying upon observed phenotypic variation, in order to infer underlying genetic causes for a given trait. Rather, next generation plant breeding may include the utilization of molecular markers and marker assisted selection (MAS), such that the breeder can directly observe movement of alleles and genetic elements of interest from one plant in the breeding population to another, and is not confined to merely observing phenotype. Further, next generation plant breeding methods are not confined to utilizing natural genetic variation found within a plant population. Rather, the breeder utilizing next generation plant breeding methodology can access a host of modern genetic engineering tools that directly alter/change/edit the plant's underlying genetic architecture in a targeted manner, in order to bring about a phenotypic trait of interest. In aspects, the plants bred with a next generation plant breeding methodology are indistinguishable from a plant that was bred in a traditional manner, as the resulting end product plant could theoretically be developed by either method. In particular aspects, a next generation plant breeding methodology may result in a plant that comprises: a genetic modification that is a deletion or insertion of any size; a genetic modification that is one or more base pair substitution; a genetic modification that is an introduction of nucleic acid sequences from within the plant's natural gene pool (e.g. any plant that could be crossed or bred with a plant of interest) or from editing of nucleic acid sequences in a plant to correspond to a sequence known to occur in the plant's natural gene pool; and offspring of said plants.

As used herein, the term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., that the coding sequence is under the transcriptional control of the promoter). Coding sequences can be operably linked to regulatory sequences in a sense or antisense orientation. In another example, the complementary RNA regions of the disclosure can be operably linked, either directly or indirectly, 5′ to the target mRNA, or 3′ to the target mRNA, or within the target mRNA, or a first complementary region is 5′ and its complement is 3′ to the target mRNA.

The terms “polynucleotide,” “nucleic acid,” and “nucleotide sequence,” used interchangeably herein, refers to a polymeric form of nucleotides of any length, either ribonucleotides or deoxyribonucleotides, or analogs thereof. This term refers to the primary structure of the molecule, and thus includes double- and single-stranded DNA, as well as double- and single-stranded RNA. This term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. It also includes modified nucleic acids such as methylated and/or capped nucleic acids, nucleic acids containing modified bases, backbone modifications, and the like. “Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as “oligomers” or “oligos” and may be isolated from genes, or chemically synthesized by methods known in the art. The terms “polynucleotide” “nucleic acid,” and “nucleotide sequence” should be understood to include, as applicable to the embodiments being described, single-stranded (such as sense or antisense) and double-stranded polynucleotides.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

As used herein, the phrases “recombinant construct”, “expression construct”, “chimeric construct”, “construct”, and “recombinant DNA construct” are used interchangeably herein. A recombinant construct comprises an artificial combination of nucleic acid fragments, e.g., regulatory and coding sequences that are not found together in nature. For example, a chimeric construct may comprise regulatory sequences and coding sequences that are derived from different sources, or regulatory sequences and coding sequences derived from the same source, but arranged in a manner different than that found in nature. Such construct may be used by itself or may be used in conjunction with a vector. If a vector is used then the choice of vector is dependent upon the method that will be used to transform host cells as is well known to those skilled in the art. For example, a plasmid vector can be used. The skilled artisan is well aware of the genetic elements that must be present on the vector in order to successfully transform, select and propagate host cells comprising any of the isolated nucleic acid fragments of the disclosure. The skilled artisan will also recognize that different independent transformation events will result in different levels and patterns of expression (Jones et al., (1985) EMBO J. 4:2411-2418; De Almeida et al., (1989) Mol. Gen. Genetics 218:78-86), and thus that multiple events must be screened in order to obtain lines displaying the desired expression level and pattern. Such screening may be accomplished by Southern analysis of DNA, Northern analysis of mRNA expression, immunoblotting analysis of protein expression, or phenotypic analysis, among others. Vectors can be plasmids, viruses, bacteriophages, pro-viruses, phagemids, transposons, artificial chromosomes, and the like, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that is not autonomously replicating. As used herein, the term “expression” refers to the production of a functional end-product e.g., an mRNA or a protein (precursor or mature).

The term “traditional plant breeding” refers to the utilization of natural variation found within a plant population as a source for alleles and genetic variants that impart a trait of interest to a given plant. Traditional breeding methods make use of crossing procedures that rely largely upon observed phenotypic variation to infer causative allele association. That is, traditional plant breeding relies upon observations of expressed phenotype of a given plant to infer underlying genetic cause. These observations are utilized to inform the breeding procedure in order to move allelic variation into germplasm of interest. Further, traditional plant breeding has also been characterized as comprising random mutagenesis techniques, which can be used to introduce genetic variation into a given germplasm. These random mutagenesis techniques may include chemical and/or radiation-based mutagenesis procedures. Consequently, one key feature of traditional plant breeding, is that the breeder does not utilize a genetic engineering tool that directly alters/changes/edits the plant's underlying genetic architecture in a targeted manner, in order to introduce genetic diversity and bring about a phenotypic trait of interest.

A “CRISPR-associated effector” as used herein can thus be defined as any nuclease, nickase, or recombinase associated with the CRISPR (Clustered Regularly Interspaced Short Palindromic Repeats), having the capacity to introduce a single- or double-strand cleavage into a genomic target site, or having the capacity to introduce a targeted modification, including a point mutation, an insertion, or a deletion, into a genomic target site of interest. At least one CRISPR-associated effector can act on its own, or in combination with other molecules as part of a molecular complex. The CRISPR-associated effector can be present as fusion molecule, or as individual molecules associating by or being associated by at least one of a covalent or non-covalent interaction with gRNA and/or target site so that the components of the CRISPR-associated complex are brought into close physical proximity.

The term “Cas9 nuclease” and “Cas9” can be used interchangeably herein, which refer to a RNA-guided DNA endonuclease enzyme associated with the CRISPR (Clustered Regularly Interspaced Short Palindromic Repeats), including the Cas9 protein or fragments thereof (such as a protein comprising an active DNA cleavage domain of Cas9 and/or a gRNA binding domain of Cas9). Cas9 is a component of the CRISPR/Cas genome editing system, which targets and cleaves a DNA target sequence to form a DNA double strand breaks (DSB) under the guidance of a guide RNA.

The term “CRISPR RNA” or “crRNA” refers to the RNA strand responsible for hybridizing with target DNA sequences, and recruiting CRISPR endonucleases and/or CRISPR-associated effectors. crRNAs may be naturally occurring, or may be synthesized according to any known method of producing RNA.

The term “tracrRNA” refers to a small trans-encoded RNA. TracrRNA is complementary to and base pairs with crRNA to form a crRNA/tracrRNA hybrid, capable of recruiting CRISPR endonucleases and/or CRISPR-associated effectors to target sequences.

The term “Guide RNA” or “gRNA” as used herein refers to an RNA sequence or combination of sequences capable of recruiting a CRISPR endonuclease and/or CRISPR-associated effectors to a target sequence. Typically gRNA is composed of crRNA and tracrRNA molecules forming complexes through partial complement, wherein crRNA comprises a sequence that is sufficiently complementary to a target sequence for hybridization and directs the CRISPR complex (i.e. Cas9-crRNA/tracrRNA hybrid) to specifically bind to the target sequence. Also, single guide RNA (sgRNA) can be designed, which comprises the characteristics of both crRNA and tracrRNA. Therefore, as used herein, a guide RNA can be a natural or synthetic crRNA (e.g., for Cpf1), a natural or synthetic crRNA/tracrRNA hybrid (e.g., for Cas9), or a single-guide RNA (sgRNA).

The term “guide sequence” or “spacer sequence” refers to the portion of a crRNA or guide RNA (gRNA) that is responsible for hybridizing with the target DNA.

The term “protospacer” refers to the DNA sequence targeted by a guide sequence of crRNA or gRNA. In some embodiments, the protospacer sequence hybridizes with the crRNA or gRNA guide (spacer) sequence of a CRISPR complex.

The term “CRISPR landing site” as used herein, refers to a DNA sequence capable of being targeted by a CRISPR-Cas complex. In some embodiments, a CRISPR landing site comprises a proximately placed protospacer/Protopacer Adjacent Motif combination sequence that is capable of being cleaved by a CRISPR complex.

The term “CRISPR complex”, “CRISPR endonuclease complex”, “CRISPR Cas complex”, or “CRISPR-gRNA complex” are used interchangeably herein. “CRISPR complex” refers to a Cas9 nuclease and/or a CRISPR-associated effectors complexed with a guide RNA (gRNA). The term “CRISPR complex” thus refers to a combination of CRISPR endonuclease and guide RNA capable of inducing a double stranded break at a CRISPR landing site. In some embodiments, “CRISPR complex” of the present disclosure refers to a combination of catalytically dead Cas9 protein and guide RNA capable of targeting a target sequence, but not capable of inducing a double stranded break at a CRISPR landing site because it loses a nuclease activity. In other embodiments, “CRISPR complex” of the present disclosure refers to a combination of Cas9 nickase and guide RNA capable of introducing gRNA-targeted single-strand breaks in DNA instead of the double-strand breaks created by wild type Cas enzymes.

As used herein, the term “directing sequence-specific binding” in the context of CRISPR complexes refers to a guide RNA's ability to recruit a CRISPR endonuclease and/or a CRISPR-associated effectors to a CRISPR landing site.

As used herein the term “targeted” refers to the expectation that one item or molecule will interact with another item or molecule with a degree of specificity, so as to exclude non-targeted items or molecules. For example, a first polynucleotide that is targeted to a second polynucleotide, according to the present disclosure has been designed to hybridize with the second polynucleotide in a sequence specific manner (e.g., via Watson-Crick base pairing). In some embodiments, the selected region of hybridization is designed so as to render the hybridization unique to the one, or more targeted regions. A second polynucleotide can cease to be a target of a first targeting polynucleotide, if its targeting sequence (region of hybridization) is mutated, or is otherwise removed/separated from the second polynucleotide.

Furthermore, “targeted” can be interchangeably used with “site-specific” or “site-directed,” which refers to an action of molecular biology which uses information on the sequence of a genomic region of interest to be modified, and which further relies on information of the mechanism of action of molecular tools, e.g., nucleases, including CRISPR nucleases and variants thereof, TALENs, ZFNs, meganucleases or recombinases, DNA-modifying enzymes, including base modifying enzymes like cytidine deaminase enzymes, histone modifying enzymes and the like, DNA-binding proteins, cr/tracr RNAs, guide RNAs and the like.

The term “seed region” refers to the critical portion of a crRNA's or guide RNA's guide sequence that is most susceptible to mismatches with their targets. In some embodiments, a single mismatch in the seed region of a crRNA/gRNA can render a CRISPR complex inactive at that binding site. In some embodiments, the seed regions for Cas9 endonucleases are located along the last ˜12 nts of the 3′ portion of the guide sequence, which correspond (hybridize) to the portion of the protospacer target sequence that is adjacent to the PAM. In some embodiments, the seed regions for Cpf1 endonucleases are located along the first ˜5 nts of the 5′ portion of the guide sequence, which correspond (hybridize) to the portion of the protospacer target sequence adjacent to the PAM.

The term “sequence identity” refers to the percentage of bases or amino acids between two polynucleotide or polypeptide sequences that are the same, and in the same relative position. As such one polynucleotide or polypeptide sequence has a certain percentage of sequence identity compared to another polynucleotide or polypeptide sequence. For sequence comparison, typically one sequence acts as a reference sequence, to which test sequences are compared. The term “reference sequence” refers to a molecule to which a test sequence is compared. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity.” Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4:11-17 (1988).

“Complementary” refers to the capacity for pairing, through base stacking and specific hydrogen bonding, between two sequences comprising naturally or non-naturally occurring bases or analogs thereof. For example, if a base at one position of a nucleic acid is capable of hydrogen bonding with a base at the corresponding position of a target, then the bases are considered to be complementary to each other at that position. Nucleic acids can comprise universal bases, or inert abasic spacers that provide no positive or negative contribution to hydrogen bonding. Base pairings may include both canonical Watson-Crick base pairing and non-Watson-Crick base pairing (e.g., Wobble base pairing and Hoogsteen base pairing). It is understood that for complementary base pairings, adenosine-type bases (A) are complementary to thymidine-type bases (T) or uracil-type bases (U), that cytosine-type bases (C) are complementary to guanosine-type bases (G), and that universal bases such as such as 3-nitropyrrole or 5-nitroindole can hybridize to and are considered complementary to any A, C, U, or T. Nichols et al., Nature, 1994; 369:492-493 and Loakes et al., Nucleic Acids Res., 1994; 22:4039-4043. Inosine (I) has also been considered in the art to be a universal base and is considered complementary to any A, C, U, or T. See Watkins and Santa Lucia, Nucl. Acids Research, 2005; 33 (19): 6258-6267.

As referred to herein, a “complementary nucleic acid sequence” is a nucleic acid sequence comprising a sequence of nucleotides that enables it to non-covalently bind to another nucleic acid in a sequence-specific, antiparallel, manner (i.e., a nucleic acid specifically binds to a complementary nucleic acid) under the appropriate in vitro and/or in vivo conditions of temperature and solution ionic strength.

Methods of sequence alignment for comparison and determination of percent sequence identity and percent complementarity are well known in the art. Optimal alignment of sequences for comparison can be conducted, e.g., by the homology alignment algorithm of Needleman and Wunsch, (1970) J. Mol. Biol. 48:443, by the search for similarity method of Pearson and Lipman, (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), by manual alignment and visual inspection (see, e.g., Brent et al., (2003) Current Protocols in Molecular Biology), by use of algorithms know in the art including the BLAST and BLAST 2.0 algorithms, which are described in Altschul et al., (1977) Nuc. Acids Res. 25:3389-3402; and Altschul et al., (1990) J. Mol. Biol. 215:403-410, respectively. Software for performing BLAST analyses is publicly available through the National Center for Biotechnology Information. Some alignment programs are MacVector (Oxford Molecular Ltd, Oxford, U.K.), ALIGN Plus (Scientific and Educational Software, Pennsylvania) and AlignX (Vector NTI, Invitrogen, Carlsbad, CA). Another alignment program is Sequencher (Gene Codes, Ann Arbor, Michigan), using default parameters, and MUSCLE (Multiple Sequence Comparison by Log-Expection; a computer software licensed as public domain).

Herein, the term “hybridize” refers to pairing between complementary nucleotide bases (e.g., adenine (A) forms a base pair with thymine (T) in a DNA molecule and with uracil (U) in an RNA molecule, and guanine (G) forms a base pair with cytosine (C) in both DNA and RNA molecules) to form a double-stranded nucleic acid molecule. (See, e.g., Wahl and Berger (1987) Methods Enzymol. 152:399; Kimmel, (1987) Methods Enzymol. 152:507). In addition, it is also known in the art that for hybridization between two RNA molecules (e.g., dsRNA), guanine (G) base pairs with uracil (U). For example, G/U base-pairing is partially responsible for the degeneracy (i.e., redundancy) of the genetic code in the context of tRNA anti-codon base-pairing with codons in mRNA. In the context of this disclosure, a guanine (G) of a protein-binding segment (dsRNA duplex) of a guide RNA molecule is considered complementary to a uracil (U), and vice versa. As such, when a G/U base-pair can be made at a given nucleotide position a protein-binding segment (dsRNA duplex) of a guide RNA molecule, the position is not considered to be non-complementary, but is instead considered to be complementary. It is understood in the art that the sequence of polynucleotide need not be 100% complementary to that of its target nucleic acid to be specifically hybridizable. Moreover, a polynucleotide may hybridize over one or more segments such that intervening or adjacent segments are not involved in the hybridization event (e.g., a loop structure or hairpin structure). A polynucleotide can comprise at least 70%, at least 80%, at least 90%, at least 95%, at least 99%, or 100% sequence complementarity to a target region within the target nucleic acid sequence to which they are targeted.

The term “modified” refers to a substance or compound (e.g., a cell, a polynucleotide sequence, and/or a polypeptide sequence) that has been altered or changed as compared to the corresponding unmodified substance or compound.

“Isolated” refers to a material that is free to varying degrees from components which normally accompany it as found in its native state.

The term “gene edited plant, part or cell” as used herein refers to a plant, part or cell that comprises one or more endogenous genes that are edited by a gene editing system. The gene editing system of the present disclosure comprises a targeting element and/or an editing element. The targeting element is capable of recognizing a target genomic sequence. The editing element is capable of modifying the target genomic sequence, e.g., by substitution or insertion of one or more nucleotides in the genomic sequence, deletion of one or more nucleotides in the genomic sequence, alteration of genomic sequences to include regulatory sequences, insertion of transgenes at a safe harbor genomic site or other specific location in the genome, or any combination thereof. The targeting element and the editing element can be on the same nucleic acid molecule or different nucleic acid molecules.

The term “plant part” includes differentiated and undifferentiated tissues including, but not limited to: plant organs, plant tissues, roots, stems, shoots, rootstocks, scions, stipules, petals, leaves, flowers, ovules, pollens, bracts, petioles, internodes, bark, pubescence, tillers, rhizomes, fronds, blades, stamens, fruits, seeds, tumor tissue and plant cells (e.g., single cells, protoplasts, embryos, and callus tissue). Plant cells include, without limitation, cells from seeds, suspension cultures, embryos, meristematic regions, callus tissue, leaves, roots, shoots, gametophytes, sporophytes, pollen and microspores. The plant tissue may be in a plant or in a plant organ, tissue or cell culture.

As used herein when discussing plants, the term “ovule” refers to the female gametophyte, whereas the term “pollen” means the male gametophyte.

As used herein, the term “plant tissue” refers to any part of a plant. Examples of plant organs include, but are not limited to the leaf, stem, root, tuber, seed, branch, pubescence, nodule, leaf axil, flower, pollen, stamen, pistil, petal, peduncle, stalk, stigma, style, bract, fruit, trunk, carpel, sepal, anther, ovule, pedicel, needle, cone, rhizome, stolon, shoot, pericarp, endosperm, placenta, berry, stamen, and leaf sheath.

As used herein, the term “phenotype” refers to the observable characters of an individual cell, cell culture, organism (e.g., a plant), or group of organisms which results from the interaction between that individual's genetic makeup (i.e., genotype) and the environment.

The terms “transgene” or “transgenic” as used herein refer to at least one nucleic acid sequence that is taken from the genome of one organism, or produced synthetically, and which is then introduced into a host cell or organism or tissue of interest and which is subsequently integrated into the host's genome by means of “stable” transformation or transfection approaches. In contrast, the term “transient” transformation or transfection or introduction refers to a way of introducing molecular tools including at least one nucleic acid (DNA, RNA, single-stranded or double-stranded or a mixture thereof) and/or at least one amino acid sequence, optionally comprising suitable chemical or biological agents, to achieve a transfer into at least one compartment of interest of a cell, including, but not restricted to, the cytoplasm, an organelle, including the nucleus, a mitochondrion, a vacuole, a chloroplast, or into a membrane, resulting in transcription and/or translation and/or association and/or activity of the at least one molecule introduced without achieving a stable integration or incorporation and thus inheritance of the respective at least one molecule introduced into the genome of a cell. The terms “transgene-free” refers to a condition that transgene is not present or found in the genome of a host cell or tissue or organism of interest.

As used herein, the term “tissue culture” indicates a composition comprising isolated cells of the same or a different type or a collection of such cells organized into parts of a plant.

Exemplary types of tissue cultures are protoplasts, calli, plant clumps, and plant cells that can generate tissue culture that are intact in plants or parts of plants, such as embryos, pollen, flowers, seeds, leaves, stems, roots, root tips, anthers, pistils, meristematic cells, axillary buds, ovaries, seed coat, endosperm, hypocotyls, cotyledons and the like. The term “plant organ” refers to plant tissue or a group of tissues that constitute a morphologically and functionally distinct part of a plant. “Progeny” comprises any subsequent generation of a plant.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

By “biologically active portion” is meant a portion of a full-length parent peptide or polypeptide which portion retains an activity of the parent molecule. As used herein, the term “biologically active portion” includes deletion mutants and peptides, for example of at least about 8, 9, 10, 11, 12, 13, 14, 15, 16, 17, 18, 19, 20, 21, 22, 23, 24, 25, 30, 40, 50, 60, 70, 80, 90, 100, 120, 150, 300, 400, 500, 600, 700, 800, 900 or 1000 contiguous amino acids, which comprise an activity of a parent molecule. Portions of this type may be obtained through the application of standard recombinant nucleic acid techniques or synthesized using conventional liquid or solid phase synthesis techniques. For example, reference may be made to solution synthesis or solid phase synthesis as described, for example, in Chapter 9 entitled “Peptide Synthesis” by Atherton and Shephard which is included in a publication entitled “Synthetic Vaccines” edited by Nicholson and published by Blackwell Scientific Publications. Alternatively, peptides can be produced by digestion of a peptide or polypeptide of the disclosure with proteinases such as endoLys-C, endoArg-C, endoGlu-C and staphylococcus V8-protease. The digested fragments can be purified by, for example, high performance liquid chromatographic (HPLC) techniques. Recombinant nucleic acid techniques can also be used to produce such portions.

By “corresponds to” or “corresponding to” is meant a polynucleotide (a) having a nucleotide sequence that is substantially identical or complementary to all or a portion of a reference polynucleotide sequence or (b) encoding an amino acid sequence identical to an amino acid sequence in a peptide or protein. This phrase also includes within its scope a peptide or polypeptide having an amino acid sequence that is substantially identical to a sequence of amino acids in a reference peptide or protein.

The terms “growing” or “regeneration” as used herein mean growing a whole, differentiated plant from a plant cell, a group of plant cells, a plant part (including seeds), or a plant piece (e.g., from a protoplast, callus, or tissue part).

As used herein, the term “derived from” refers to the origin or source, and may include naturally occurring, recombinant, unpurified, or purified molecules. A nucleic acid or an amino acid derived from an origin or source may have all kinds of nucleotide changes or protein modification as defined elsewhere herein.

By “obtained from” is meant that a sample such as, for example, a nucleic acid extract or polypeptide extract is isolated from, or derived from, a particular source. For example, the extract may be isolated directly from plants.

By “variant” polypeptide is intended a polypeptide derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Variant proteins encompassed by the present disclosure are biologically active, that is they continue to possess the desired biological activity of the native protein, that is, modulating or regulatory activity as described herein. Such variants may result from, for example, genetic polymorphism or from human manipulation. Biologically active variants of a native R protein of the disclosure will have at least 40%, 50%, 60%, 70%, generally at least 75%, 80%, 85%, preferably about 90% to 95% or more, and more preferably about 98% or more sequence identity to the amino acid sequence for the native protein as determined by sequence alignment programs described elsewhere herein using default parameters. A biologically active variant of a protein of the disclosure may differ from that protein by as few as 1-15 amino acid residues, as few as 1-10, such as 6-10, as few as 5, as few as 4, 3, 2, or even 1 amino acid residue.

The proteins of the disclosure may be altered in various ways including amino acid substitutions, deletions, truncations, and insertions. Methods for such manipulations are generally known in the art. For example, amino acid sequence variants of the R proteins can be prepared by mutations in the DNA. Methods for mutagenesis and nucleotide sequence alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be preferable.

Individual substitutions deletions or additions that alter, add or delete a single amino acid or a small percentage of amino acids (typically less than 5%, more typically less than 1%) in an encoded sequence are “conservatively modified variations,” where the alterations result in the substitution of an amino acid with a chemically similar amino acid. Conservative substitution tables providing functionally similar amino acids are well known in the art. The following five groups each contain amino acids that are conservative substitutions for one another, Aliphatic: Glycine (G), Alanine (A), Valine (V), Leucine (L), Isoleucine (I); Aromatic: Phenylalanine (F), Tyrosine (Y), Tryptophan (W); Sulfur-containing: Methionine (M), Cysteine (C); Basic: Arginine I, Lysine (K), Histidine (H); Acidic: Aspartic acid (D), Glutamic acid (E), Asparagine (N), Glutamine (Q). See also, Creighton, 1984. In addition, individual substitutions, deletions or additions which alter, add or delete a single amino acid or a small percentage of amino acids in an encoded sequence are also “conservatively modified variations.”

“Expression cassette” as used herein means a DNA sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. The expression cassette may also be one which is naturally occurring but has been obtained in a recombinant form useful for heterologous expression. The expression of the nucleotide sequence in the expression cassette may be under the control of a constitutive promoter or of an inducible promoter which initiates transcription only when the host cell is exposed to some particular external stimulus. In the case of a multicellular organism, the promoter can also be specific to a particular tissue or organ or stage of development in animal and/or plant.

As used herein, the term “vector”, “plasmid”, or “construct” refers broadly to any plasmid or virus encoding an exogenous nucleic acid. The term should also be construed to include non-plasmid and non-viral compounds which facilitate transfer of nucleic acid into virions or cells, such as, for example, polylysine compounds and the like. The vector may be a viral vector that is suitable as a delivery vehicle for delivery of the nucleic acid, or mutant thereof, to a cell, or the vector may be a non-viral vector which is suitable for the same purpose. Examples of viral and non-viral vectors for delivery of DNA to cells and tissues are well known in the art and are described, for example, in Ma et al. (1997, Proc. Natl. Acad. Sci. U.S.A. 94:12744-12746). Examples of viral vectors include, but are not limited to, recombinant plant viruses. Non-limiting examples of plant viruses include, TMV-mediated (transient) transfection into tobacco (Tuipe, T-H et al (1993), J. Virology Meth, 42: 227-239), ssDNA genomes viruses (e.g., family Geminiviridae), reverse transcribing viruses (e.g., families Caulimoviridae, Pseudoviridae, and Metaviridae), dsNRA viruses (e.g., families Reoviridae and Partitiviridae), (−) ssRNA viruses (e.g., families Rhabdoviridae and Bunyaviridae), (+) ssRNA viruses (e.g., families Bromoviridae, Closteroviridae, Comoviridae, Luteoviridae, Potyviridae, Sequiviridae and Tombusviridae) and viroids (e.g., families Pospiviroldae and Avsunviroidae). Detailed classification information of plant viruses can be found in Fauquet et al (2008, “Geminivirus strain demarcation and nomenclature”. Archives of Virology 153:783-821, incorporated herein by reference in its entirety), and Khan et al. (Plant viruses as molecular pathogens; Publisher Routledge, 2002, ISBN 1560228954, 9781560228950). Examples of non-viral vectors include, but are not limited to, liposomes, polyamine derivatives of DNA, and the like.

Also, “vector” is defined to include, inter alia, any plasmid, cosmid, phage or Agrobacterium binary vector in double or single stranded linear or circular form which may or may not be self-transmissible or mobilizable, and which can transform prokaryotic or eukaryotic host either by integration into the cellular genome or exist extrachromosomally (e.g. autonomous replicating plasmid with an origin of replication).

Specifically included are shuttle vectors by which is meant a DNA vehicle capable, naturally or by design, of replication in two different host organisms, which may be selected from actinomycetes and related species, bacteria and eukaryotic (e.g. higher plant, mammalian, yeast or fungal cells).

Preferably the nucleic acid in the vector is under the control of, and operably linked to, an appropriate promoter or other regulatory elements for transcription in a host cell such as a microbial, e.g. bacterial, or plant cell. The vector may be a bi-functional expression vector which functions in multiple hosts. In the case of genomic DNA, this may contain its own promoter or other regulatory elements and in the case of cDNA this may be under the control of an appropriate promoter or other regulatory elements for expression in the host cell.

“Cloning vectors” typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide tetracycline resistance, hygromycin resistance or ampicillin resistance.

As used herein, the term “offspring” refers to any plant resulting as progeny from a vegetative or sexual reproduction from one or more parent plants or descendants thereof. For instance an offspring plant may be obtained by cloning or selfing of a parent plant or by crossing two parents plants and include selfings as well as the F1 or F2 or still further generations. An F1 is a first-generation offspring produced from parents at least one of which is used for the first time as donor of a trait, while offspring of second generation (F2) or subsequent generations (F3, F4, etc.) are specimens produced from selfings of F1's, F2's etc. An F1 may thus be (and usually is) a hybrid resulting from a cross between two true breeding parents (true-breeding is homozygous for a trait), while an F2 may be (and usually is) an offspring resulting from self-pollination of said F1 hybrids.

The term “plant” includes reference to whole plants, plant organs, plant tissues, and plant cells and progeny of same, but is not limited to angiosperms and gymnosperms such as Arabidopsis, potato, tomato, tobacco, alfalfa, lettuce, carrot, strawberry, sugarbeet, cassava, sweet potato, soybean, lima bean, pea, chick pea, maize (corn), turf grass, wheat, rice, barley, sorghum, oat, oak, eucalyptus, walnut, palm and duckweed as well as fern and moss. Thus, a plant may be a monocot, a dicot, a vascular plant reproduced from spores such as fern or a non-vascular plant such as moss, liverwort, hornwort and algae. The word “plant,” as used herein, also encompasses plant cells, seed, plant progeny, propagule whether generated sexually or asexually, and descendants of any of these, such as cuttings or seed. Plant cells include suspension cultures, callus, embryos, meristematic regions, callus tissue, leaves, roots, shoots, gametophytes, sporophytes, pollen, seeds and microspores. Plants may be at various stages of maturity and may be grown in liquid or solid culture, or in soil or suitable media in pots, greenhouses or fields. Expression of an introduced leader, trailer or gene sequences in plants may be transient or permanent. A “selected plant species” may be, but is not limited to, a species of any one of these “plants.”

In the present disclosure, the plants are intended to comprise without limitation angiosperm and gymnosperm plants such as acacia, alfalfa, amaranth, apple, apricot, artichoke, ash tree, asparagus, avocado, banana, barley, beans, beet, birch, beech, blackberry, black raspberry, blueberry, broccoli, Brussel's sprouts, cabbage, cane berry, canola, cantaloupe, carrot, cassava, cauliflower, cedar, a cereal, celery, chestnut, cherry, Chinese cabbage, citrus, Clementine, clover, coffee, corn, cotton, cowpea, cucumber, cypress, eggplant, elm, endive, eucalyptus, fennel, figs, fir, geranium, grape, grapefruit, groundnuts, ground cherry, gum hemlock, hickory, kale, kiwifruit, kohlrabi, larch, lettuce, leek, lemon, lime, locust, pine, maidenhair, maize, mango, maple, melon, millet, mushroom, mustard, nuts, oak, oats, oil palm, okra, onion, orange, an ornamental plant or flower or tree, papaya, palm, parsley, parsnip, pea, peach, peanut, pear, peat, pepper, persimmon, pigeon pea, peach, pine, pineapple, plantain, plum, pomegranate, potato, pumpkin, radicchio, radish, rapeseed, raspberry, rice, rye, sorghum, safflower, sallow, soybean, spinach, spruce, squash, strawberry, sugar beet, sugarcane, sunflower, sweet potato, sweet corn, tangerine, tea, tobacco, tomato, trees, triticale, turf grasses, turnips, vine, walnut, watercress, watermelon, wheat, wild strawberry, yams, yew, and zucchini.

Angiosperm is defined as vascular plants having seeds enclosed in an ovary. Angiosperms are seed plants that produce flowers that bear fruits. Angiosperms are divided into dicotyledonous and monocotyledonous plants.

Dicotyledonous plant (Dicot) is defined as a flowering plant whose embryos have two seed halves or cotyledons, branching leaf veins, and flower parts in multiples of four or five. Examples of dicots include but are not limited to, Eucalyptus, Populus, Liquidamber, Acacia, teak, mahogany, tobacco, Arabidopsis, tomato, potato sugar beet, broccoli, cassava, sweet potato, pepper, poinsettia, bean, rapeseed/canola, alfalfa, radish, crimson clover, field pennycress, soybean, carrot, strawberry, lettuce, oak, maple, walnut, rose, mint, squash, daisy, geranium, avocado, cotton/cottonseed and cactus.

Thlaspi arvense, known by the common name field pennycress (aka pennycress), is a flowering plant in the cabbage family Brassicaceae. CoverCress is a new oilseed crop grown over winter between normal full season corn and soybeans. CoverCress was developed from pennycress. Low fiber pennycress lines are provided in U.S. Pat. No. 10,709,151, which is assigned to CoverCress Inc.

Monocotyledonous Plant (Monocot) is defined as a flowering plant having embryos with one cotyledon or seed leaf, parallel leaf veins, and flower parts in multiples of three. Examples of monocots include, but are not limited to turfgrass, corn/maize, rice, oat, annual ryegrass, wheat, barley, sorghum, orchid, iris, lily, onion, and palm. Examples of turfgrass include, but are not limited to Agrostis spp. (bentgrass species including colonial bentgrass and creeping bentgrasses), Poa pratensis (Kentucky bluegrass), Lolium spp. (ryegrass species including annual ryegrass and perennial ryegrass), Festuca arundinacea (tall fescue) Festuca rubra commutata (Chewings fescue), Cynodon dactylon (bermudagrass, Pennisetum clandestinum (kikuyu grass), Stenotaphrum secundatum (St. Augustine grass), Zoysia japonica (zoysia grass), and Dichondra micrantha.

The methods for targeted gene-editing system can be used to confer desired traits on essentially any plant. A wide variety of plants and plant cell systems may be engineered for the desired physiological and agronomic characteristics described herein using the nucleic acid constructs of the present disclosure and the various transformation methods. In preferred embodiments, target plants and plant cells for engineering include, but are not limited to, those monocotyledonous and dicotyledonous plants, such as crops including grain crops (e.g., wheat, maize, rice, millet, barley), fruit crops (e.g., tomato, apple, grape, peach, pear, plum, raspberry, black raspberry, blackberry, cane berry, cherry, avocado, strawberry, wild strawberry, orange), forage crops (e.g., alfalfa), root vegetable crops (e.g., carrot, potato, sugar beets, yam), leafy vegetable crops (e.g., lettuce, spinach); flowering plants (e.g., petunia, rose, chrysanthemum), conifers and pine trees (e.g., pine fir, spruce); plants used in phytoremediation (e.g., heavy metal accumulating plants); oil crops (e.g., sunflower, rape seed) and plants used for experimental purposes (e.g., Arabidopsis). In some embodiments, fruit crops such as tomato, apple, peach, pear, plum, raspberry, black raspberry, blackberry, cane berry, cherry, avocado, strawberry, wild strawberry, grape and orange.

As used herein, the term “gene” refers to any segment of DNA associated with a biological function. Thus, genes include, but are not limited to, coding sequences and/or the regulatory sequences required for their expression. Genes can also include nonexpressed DNA segments that, for example, form recognition sequences for other proteins. Genes can be obtained from a variety of sources, including cloning from a source of interest or synthesizing from known or predicted sequence information, and may include sequences designed to have desired parameters.

As used herein, the term “genotype” refers to the genetic makeup of an individual cell, cell culture, tissue, organism (e.g., a plant), or group of organisms.

As used herein, the term “allele(s)” means any of one or more alternative forms of a gene, all of which alleles relate to at least one trait or characteristic. In a diploid cell, the two alleles of a given gene occupy corresponding loci on a pair of homologous chromosomes. Since the present disclosure relates to QTLs, i.e. genomic regions that may comprise one or more genes or regulatory sequences, it is in some instances more accurate to refer to “haplotype” (i.e. an allele of a chromosomal segment) instead of “allele”, however, in those instances, the term “allele” should be understood to comprise the term “haplotype”. Alleles are considered identical when they express a similar phenotype. Differences in sequence are possible but not important as long as they do not influence phenotype.

As used herein, the term “locus” (plural: “loci”) refers to any site that has been defined genetically. A locus may be a gene, or part of a gene, or a DNA sequence that has some regulatory role, and may be occupied by different sequences.

As used herein, the term “molecular marker” or “genetic marker” refers to an indicator that is used in methods for visualizing differences in characteristics of nucleic acid sequences. Examples of such indicators are restriction fragment length polymorphism (RFLP) markers, amplified fragment length polymorphism (AFLP) markers, single nucleotide polymorphisms (SNPs), insertion mutations, microsatellite markers (SSRs), sequence-characterized amplified regions (SCARs), cleaved amplified polymorphic sequence (CAPS) markers or isozyme markers or combinations of the markers described herein which defines a specific genetic and chromosomal location. Mapping of molecular markers in the vicinity of an allele is a procedure which can be performed quite easily by the average person skilled in molecular-biological techniques which techniques are for instance described in Lefebvre and Chevre, 1995; Lorez and Wenzel, 2007, Srivastava and Narula, 2004, Meksem and Kahl, 2005, Phillips and Vasil, 2001. General information concerning AFLP technology can be found in Vos et al. (1995, AFLP: a new technique for DNA fingerprinting, Nucleic Acids Res. 1995 Nov. 11; 23(21): 4407-4414).

As used herein, the term “hemizygous” refers to a cell, tissue or organism in which a gene is present only once in a genotype, as a gene in a haploid cell or organism, a sex-linked gene in the heterogametic sex, or a gene in a segment of chromosome in a diploid cell or organism where its partner segment has been deleted.

As used herein, the term “heterozygote” refers to a diploid or polyploid individual cell or plant having different alleles (forms of a given gene) present at least at one locus.

As used herein, the term “heterozygous” refers to the presence of different alleles (forms of a given gene) at a particular gene locus.

As used herein, the term “homozygote” refers to an individual cell or plant having the same alleles at one or more loci.

As used herein, the term “homozygous” refers to the presence of identical alleles at one or more loci in homologous chromosomal segments.

As used herein, the term “homologous” or “homolog” is known in the art and refers to related sequences that share a common ancestor or family member and are determined based on the degree of sequence identity. The terms “homology”, “homologous”, “substantially similar” and “corresponding substantially” are used interchangeably herein. Homologs usually control, mediate, or influence the same or similar biochemical pathways, yet particular homologs may give rise to differing phenotypes. It is therefore understood, as those skilled in the art will appreciate, that the disclosure encompasses more than the specific exemplary sequences. These terms describe the relationship between a gene found in one species, subspecies, variety, cultivar or strain and the corresponding or equivalent gene in another species, subspecies, variety, cultivar or strain. For purposes of this disclosure homologous sequences are compared.

The term “homolog” is sometimes used to apply to the relationship between genes separated by the event of speciation (see “ortholog”) or to the relationship between genes separated by the event of genetic duplication (see “paralog”).

The term “homeolog” refers to a homeologous gene or chromosome, resulting from polyploidy or chromosomal duplication events. This contrasts with the more common ‘homolog’, which is defined immediately above.

The term “ortholog” refers to genes in different species that evolved from a common ancestral gene by speciation. Normally, orthologs retain the same function in the course of evolution. Identification of orthologs is critical for reliable prediction of gene function in newly sequenced genomes.

The term “paralog” refers to genes related by duplication within a genome. While orthologs generally retain the same function in the course of evolution, paralogs can evolve new functions, even if these are related to the original one.

“Homologous sequences” or “homologs” or “orthologs” are thought, believed, or known to be functionally related. A functional relationship may be indicated in any one of a number of ways, including, but not limited to: (a) degree of sequence identity and/or (b) the same or similar biological function. Preferably, both (a) and (b) are indicated. The degree of sequence identity may vary, but in one embodiment, is at least 50% (when using standard sequence alignment programs known in the art), at least 60%, at least 65%, at least 70%, at least 75%, at least 80%, at least 85%, at least 90%, at least about 91%, at least about 92%, at least about 93%, at least about 94%, at least about 95%, at least about 96%, at least about 97%, at least about 98%, or at least 98.5%, or at least about 99%, or at least 99.5%, or at least 99.8%, or at least 99.9%. Homology can be determined using software programs readily available in the art, such as those discussed in Current Protocols in Molecular Biology (F. M. Ausubel et al., eds., 1987) Supplement 30, section 7.718, Table 7.71. Some alignment programs are MacVector (Oxford Molecular Ltd, Oxford, U.K.) and ALIGN Plus (Scientific and Educational Software, Pennsylvania). Other non-limiting alignment programs include Sequencher (Gene Codes, Ann Arbor, Michigan), AlignX, and Vector NTI (Invitrogen, Carlsbad, CA).

As used herein, the term “hybrid” refers to any individual cell, tissue or plant resulting from a cross between parents that differ in one or more genes.

As used herein, the term “inbred” or “inbred line” refers to a relatively true-breeding strain.

The term “single allele converted plant” as used herein refers to those plants which are developed by a plant breeding technique called backcrossing wherein essentially all of the desired morphological and physiological characteristics of an inbred are recovered in addition to the single allele transferred into the inbred via the backcrossing technique.

As used herein, the term “line” is used broadly to include, but is not limited to, a group of plants vegetatively propagated from a single parent plant, via tissue culture techniques or a group of inbred plants which are genetically very similar due to descent from a common parent(s). A plant is said to “belong” to a particular line if it (a) is a primary transformant (TO) plant regenerated from material of that line; (b) has a pedigree comprised of a TO plant of that line; or (c) is genetically very similar due to common ancestry (e.g., via inbreeding or selfing). In this context, the term “pedigree” denotes the lineage of a plant, e.g. in terms of the sexual crosses affected such that a gene or a combination of genes, in heterozygous (hemizygous) or homozygous condition, imparts a desired trait to the plant.

As used herein, the terms “wildtype check”, “wildtype” or “check” all refer to a first cell, tissue culture, part or organism which is essentially genetically the same as a second cell, tissue culture, part or organism, respectively, except that the corresponding second cell, tissue culture, part or organism comprises a heterologous genetic element not present in the first cell, tissue culture, part or organism. Thus, for example, a first plant would be a wildtype check relative to a second plant where the only meaningful genetic difference between the two is that the second plant comprises a heterologous gene not present in the first plant.

As used herein, the terms “introgression”, “introgressed” and “introgressing” refer to the process whereby genes of one species, variety or cultivar are moved into the genome of another species, variety or cultivar, by crossing those species. The crossing may be natural or artificial. The process may optionally be completed by backcrossing to the recurrent parent, in which case introgression refers to infiltration of the genes of one species into the gene pool of another through repeated backcrossing of an interspecific hybrid with one of its parents. An introgression may also be described as a heterologous genetic material stably integrated in the genome of a recipient plant.

As used herein, the term “population” means a genetically homogeneous or heterogeneous collection of plants sharing a common genetic derivation.

As used herein, the term “variety” or “cultivar” means a group of similar plants that by structural features and performance can be identified from other varieties within the same species. The term “variety” as used herein has identical meaning to the corresponding definition in the International Convention for the Protection of New Varieties of Plants (UPOV treaty), of Dec. 2, 1961, as Revised at Geneva on Nov. 10, 1972, on Oct. 23, 1978, and on Mar. 19, 1991. Thus, “variety” means a plant grouping within a single botanical taxon of the lowest known rank, which grouping, irrespective of whether the conditions for the grant of a breeder's right are fully met, can be i) defined by the expression of the characteristics resulting from a given genotype or combination of genotypes, ii) distinguished from any other plant grouping by the expression of at least one of the said characteristics and iii) considered as a unit with regard to its suitability for being propagated unchanged.

A variety is deemed to be essentially derived from another variety (‘the initial variety’) when: (i) it is predominantly derived from the initial variety, or from a variety that is itself predominantly derived from the initial variety, while retaining the expression of the essential characteristics that result from the genotype or combination of genotypes of the initial variety; (ii) it is clearly distinguishable from the initial variety; and, (iii) except for the differences which result from the act of derivation, it conforms to the initial variety in the expression of the essential characteristics that result from the genotype or combination of genotypes of the initial variety. UPOV, Article 14(5)(b).

As used herein, the term “mass selection” refers to a form of selection in which individual plants are selected and the next generation propagated from the aggregate of their seeds. More details of mass selection are described herein in the specification.

As used herein, the term “open pollination” refers to a plant population that is freely exposed to some gene flow, as opposed to a closed one in which there is an effective barrier to gene flow.

As used herein, the terms “open-pollinated population” or “open-pollinated variety” refer to plants normally capable of at least some cross-fertilization, selected to a standard, that may show variation but that also have one or more genotypic or phenotypic characteristics by which the population or the variety can be differentiated from others. A hybrid, which has no barriers to cross-pollination, is an open-pollinated population or an open-pollinated variety.

As used herein, the term “self-crossing”, “self pollinated” or “self-pollination” means the pollen of one flower on one plant is applied (artificially or naturally) to the ovule (stigma) of the same or a different flower on the same plant.

As used herein, the term “cross”, “crossing”, “cross pollination” or “cross-breeding” refer to the process by which the pollen of one flower on one plant is applied (artificially or naturally) to the ovule (stigma) of a flower on another plant.

As used herein, the term “derived from” refers to the origin or source, and may include naturally occurring, recombinant, unpurified, or purified molecules. A nucleic acid or an amino acid derived from an origin or source may have all kinds of nucleotide changes or protein modification as defined elsewhere herein.

The term “primer” as used herein refers to an oligonucleotide which is capable of annealing to the amplification target allowing a DNA polymerase to attach, thereby serving as a point of initiation of DNA synthesis when placed under conditions in which synthesis of primer extension product is induced, i.e., in the presence of nucleotides and an agent for polymerization such as DNA polymerase and at a suitable temperature and pH. The (amplification) primer is preferably single stranded for maximum efficiency in amplification. Preferably, the primer is an oligodeoxyribonucleotide. The primer must be sufficiently long to prime the synthesis of extension products in the presence of the agent for polymerization. The exact lengths of the primers will depend on many factors, including temperature and composition (A/T and G/C content) of primer. A pair of bi-directional primers consists of one forward and one reverse primer as commonly used in the art of DNA amplification such as in PCR amplification.

A probe comprises an identifiable, isolated nucleic acid that recognizes a target nucleic acid sequence. A probe includes a nucleic acid that is attached to an addressable location, a detectable label or other reporter molecule and that hybridizes to a target sequence. Typical labels include radioactive isotopes, enzyme substrates, co-factors, ligands, chemiluminescent or fluorescent agents, haptens, and enzymes. Methods for labelling and guidance in the choice of labels appropriate for various purposes are discussed, for example, in Sambrook et al. (ed.), Molecular Cloning: A Laboratory Manual, 2^{nd} ed., vol. 1-3, Cold Spring Harbor Laboratory Press, Cold Spring Harbor, NY, 1989 and Ausubel et al. Short Protocols in Molecular Biology, 4^{th} ed., John Wiley & Sons, Inc., 1999.

Methods for preparing and using nucleic acid probes and primers are described, for example, in Sambrook et al. (ed.), Molecular Cloning: A Laboratory Manual, 2^{nd} ed., vol. 1-3, Cold Spring Harbor Laboratory Press, Cold Spring Harbor, NY, 1989; Ausubel et al. Short Protocols in Molecular Biology, 4^{th} ed., John Wiley & Sons, Inc., 1999; and Innis et al. PCR Protocols, A Guide to Methods and Applications, Academic Press, Inc., San Diego, CA, 1990. Amplification primer pairs can be derived from a known sequence, for example, by using computer programs intended for that purpose such as PRIMER (Version 0.5, 1991, Whitehead Institute for Biomedical Research, Cambridge, MA). One of ordinary skill in the art will appreciate that the specificity of a particular probe or primer increases with its length. Thus, in order to obtain greater specificity, probes and primers can be selected that comprise at least 20, 25, 30, 35, 40, 45, 50 or more consecutive nucleotides of a target nucleotide sequences.

For PCR amplifications of the polynucleotides disclosed herein, oligonucleotide primers can be designed for use in PCR reactions to amplify corresponding DNA sequences from cDNA or genomic DNA extracted from any organism of interest. Methods for designing PCR primers and PCR cloning are generally known in the art and are disclosed in Sambrook et al. (2001) Molecular Cloning: A Laboratory Manual (3rd ed., Cold Spring Harbor Laboratory Press, Plainview, New York). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

The present disclosure provides an isolated nucleic acid sequence comprising a sequence selected from the group consisting of CTR1, homologs of CTR1, orthologs of CTR1, paralogs of CTR1, and fragments and variations thereof. In one embodiment, the present disclosure provides an isolated polynucleotide encoding a protein produced by the nucleic acid sequence for CTR1, comprising a nucleic acid sequence that shares at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identity to CTR1. In some embodiments the isolated nucleic acid sequence coding for CTR1 has one or more nucleic acid insertions or deletions resulting in a frame-shift that changes the reading of subsequent codons and, therefore, alters the entire amino acid sequence that follows the mutation.

Methods of alignment of sequences for comparison are well known in the art. Various programs and alignment algorithms are described in: Smith and Waterman (Adv. Appl. Math., 2:482, 1981); Needleman and Wunsch (J. Mol. Biol., 48:443, 1970); Pearson and Lipman (Proc. Natl. Acad. Sci., 85:2444, 1988); Higgins and Sharp (Gene, 73:237-44, 1988); Higgins and Sharp (CABIOS, 5:151-53, 1989); Corpet et al. (Nuc. Acids Res., 16:10881-90, 1988); Huang et al. (Comp. Appls Biosci., 8:155-65, 1992); and Pearson et al. (Meth. Mol. Biol., 24:307-31, 1994). Altschul et al. (Nature Genet., 6:119-29, 1994) presents a detailed consideration of sequence alignment methods and homology calculations.

The present disclosure also provides a chimeric gene comprising the isolated nucleic acid sequence of any one of the polynucleotides described above operably linked to suitable regulatory sequences. In some embodiments, a chimeric gene comprises the isolated nucleic acid sequence comprising a sequence selected from the group consisting of CTR1, homologs of CTR1, orthologs of CTR1, paralogs of CTR1, and fragments and variations thereof.

In some embodiments, a chimeric gene comprises an isolated nucleic acid sequence described above, which is operably linked to suitable regulatory sequences including, but not limited to native promoters.

The present disclosure also provides a recombinant construct comprising the chimeric gene as described above. In one embodiment, said recombinant construct is a gene silencing construct, such as used in RNAi gene silencing. In another embodiment, said recombinant construct is a gene editing construct, such as used in CRISPR-Cas gene editing system.

The expression vectors of the present disclosure may include at least one selectable marker. Such markers include dihydrofolate reductase, G418 or neomycin resistance for eukaryotic cell culture and tetracycline, kanamycin or ampicillin resistance genes for culturing in E. co/i and other bacteria.

The present disclosure also provides a transformed host cell comprising the chimeric gene as described above. In one embodiment, said host cell is selected from the group consisting of bacteria, yeasts, filamentous fungi, algae, animals, and plants.

New breeding techniques (NBTs) refer to various new technologies developed and/or used to create new characteristics in plants through genetic variation, the aim being targeted mutagenesis, targeted introduction of new genes or gene silencing (RdDM). The following breeding techniques are within the scope of NBTs: targeted sequence changes facilitated through the use of Zinc finger nuclease (ZFN) technology (ZFN-1, ZFN-2 and ZFN-3, see U.S. Pat. No. 9,145,565, incorporated by reference in its entirety), Oligonucleotide directed mutagenesis (ODM, a.k.a., site-directed mutagenesis), Cisgenesis and intragenesis, epigenetic approaches such as RNA-dependent DNA methylation (RdDM, which does not necessarily change nucleotide sequence but can change the biological activity of the sequence), Grafting (on GM rootstock), Reverse breeding, Agro-infiltration for transient gene expression (agro-infiltration “sensu stricto”, agro-inoculation, floral dip), Transcription Activator-Like Effector Nucleases (TALENs, see U.S. Pat. Nos. 8,586,363 and 9,181,535, incorporated by reference in their entireties), the CRISPR/Cas system (see U.S. Pat. Nos. 8,697,359; 8,771,945; 8,795,965; 8,865,406; 8,871,445; 8,889,356; 8,895,308; 8,906,616; 8,932,814; 8,945,839; 8,993,233; and 8,999,641, which are all hereby incorporated by reference), engineered meganuclease, re-engineered homing endonucleases, DNA guided genome editing (Gao et al., Nature Biotechnology (2016), doi: 10.1038/nbt.3547, incorporated by reference in its entirety), and Synthetic genomics. A major part of today's targeted genome editing, another designation for New Breeding Techniques, is the applications to induce a DNA double strand break (DSB) at a selected location in the genome where the modification is intended. Directed repair of the DSB allows for targeted genome editing. Such applications can be utilized to generate mutations (e.g., targeted mutations or precise native gene editing) as well as precise insertion of genes (e.g., cisgenes, intragenes, or transgenes). The applications leading to mutations are often identified as site-directed nuclease (SDN) technology, such as SDN1, SDN2 and SDN3. For SDN1, the outcome is a targeted, non-specific genetic deletion mutation: the position of the DNA DSB is precisely selected, but the DNA repair by the host cell is random and results in small nucleotide deletions, additions or substitutions. For SDN2, a SDN is used to generate a targeted DSB and a DNA repair template (a short DNA sequence identical to the targeted DSB DNA sequence except for one or a few nucleotide changes) is used to repair the DSB: this results in a targeted and predetermined point mutation in the desired gene of interest. As to the SDN3, the SDN is used along with a DNA repair template that contains new DNA sequence (e.g. gene). The outcome of the technology would be the integration of that DNA sequence into the plant genome. The most likely application illustrating the use of SDN3 would be the insertion of cisgenic, intragenic, or transgenic expression cassettes at a selected genome location. A complete description of each of these techniques can be found in the report made by the Joint Research Center (JRC) Institute for Prospective Technological Studies of the European Commission in 2011 and titled “New plant breeding techniques—State-of-the-art and prospects for commercial development”, which is incorporated by reference in its entirety.

The present disclosure provides polypeptides and amino acid sequences comprising at least a portion of the CTR1 protein, homologs of CTR1, orthologs of CTR1, homeologs of CTR1, paralogs of CTR1, and fragments and variations thereof.

The present disclosure also provides an amino acid sequence of CTR1 protein, homologs of CTR1, orthologs of CTR1, paralogs of CTR1, and/or fragments and variations thereof. In some embodiments, the present disclosure provides an isolated polypeptide comprising an amino acid sequence that shares at least about 70%, about 75%, about 80%, about 85%, at least about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to an amino acid sequence of CTR1 protein, homologs of CTR1, orthologs of CTR1, paralogs of CTR1, and/or fragments and variations thereof. In one embodiment, the present disclosure provides an isolated polypeptide comprising an amino acid sequence which encodes an amino acid sequence that shares at least about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to an amino acid sequence of CTR1 (SEQ ID NO: 3).

The present disclosure provides polypeptides and amino acid sequences comprising at least a portion of the MAPK4 protein, homologs of MAPK4, orthologs of MAPK4, homeologs of MAPK4, paralogs of MAPK4, and fragments and variations thereof.

The present disclosure also provides an amino acid sequence of MAPK4 protein, homologs of MAPK4, orthologs of MAPK4, paralogs of MAPK4, and/or fragments and variations thereof. In some embodiments, the present disclosure provides an isolated polypeptide comprising an amino acid sequence that shares at least about 70%, about 75%, about 80%, about 85%, at least about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to an amino acid sequence of MAPK4 protein, homologs of MAPK4, orthologs of MAPK4, paralogs of MAPK4, and/or fragments and variations thereof. In one embodiment, the present disclosure provides an isolated polypeptide comprising an amino acid sequence which encodes an amino acid sequence that shares at least about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to an amino acid sequence of MAPK4 (SEQ ID NO: 8).

The disclosure also encompasses variants and fragments of proteins of an amino acid sequence of CTR1 protein and/or MAPK4. The variants may contain alterations in the amino acid sequences of the constituent proteins. The term “variant” with respect to a polypeptide refers to an amino acid sequence that is altered by one or more amino acids with respect to a reference sequence. The variant can have “conservative” changes, or “nonconservative” changes, e.g., analogous minor variations can also include amino acid deletions or insertions, or both.

Functional fragments and variants of a polypeptide include those fragments and variants that maintain one or more functions of the parent polypeptide. It is recognized that the gene or cDNA encoding a polypeptide can be considerably mutated without materially altering one or more of the polypeptide's functions. First, the genetic code is well-known to be degenerate, and thus different codons encode the same amino acids. Second, even where an amino acid substitution is introduced, the mutation can be conservative and have no material impact on the essential function(s) of a protein. See, e.g., Stryer Biochemistry 3rd Ed., 1988. Third, part of a polypeptide chain can be deleted without impairing or eliminating all of its functions. Fourth, insertions or additions can be made in the polypeptide chain for example, adding epitope tags, without impairing or eliminating its functions (Ausubel et al. J. Immunol. 159(5): 2502-12, 1997). Other modifications that can be made without materially impairing one or more functions of a polypeptide can include, for example, in vivo or in vitro chemical and biochemical modifications or the incorporation of unusual amino acids. Such modifications include, but are not limited to, for example, acetylation, carboxylation, phosphorylation, glycosylation, ubiquination, labelling, e.g., with radionucleotides, and various enzymatic modifications, as will be readily appreciated by those well skilled in the art. A variety of methods for labelling polypeptides, and labels useful for such purposes, are well known in the art, and include radioactive isotopes such as 32P, ligands which bind to or are bound by labelled specific binding partners (e.g., antibodies), fluorophores, chemiluminescent agents, enzymes, and anti-ligands. Functional fragments and variants can be of varying length. For example, some fragments have at least 10, 25, 50, 75, 100, 200, or even more amino acid residues. These mutations can be natural or purposely changed. In some embodiments, mutations containing alterations that produce silent substitutions, additions, or deletions, but do not alter the properties or activities of the proteins or how the proteins are made are an embodiment of the disclosure.

Conservative amino acid substitutions are those substitutions that, when made, least interfere with the properties of the original protein, that is, the structure and especially the function of the protein is conserved and not significantly changed by such substitutions. Conservative substitutions generally maintain (a) the structure of the polypeptide backbone in the area of the substitution, for example, as a sheet or helical conformation, (b) the charge or hydrophobicity of the molecule at the target site, or (c) the bulk of the side chain. Further information about conservative substitutions can be found, for instance, in Ben Bassat et al. (J. Bacteriol., 169:751 757, 1987), O'Regan et al. (Gene, 77:237 251, 1989), Sahin Toth et al. (Protein Sci., 3:240 247, 1994), Hochuli et al. (Bio/Technology, 6:1321 1325, 1988) and in widely used textbooks of genetics and molecular biology. The Blosum matrices are commonly used for determining the relatedness of polypeptide sequences. The Blosum matrices were created using a large database of trusted alignments (the BLOCKS database), in which pairwise sequence alignments related by less than some threshold percentage identity were counted (Henikoff et al., Proc. Natl. Acad. Sci. USA, 89:10915-10919, 1992). A threshold of 90% identity was used for the highly conserved target frequencies of the BLOSUM90 matrix. A threshold of 65% identity was used for the BLOSUM65 matrix. Scores of zero and above in the Blosum matrices are considered “conservative substitutions” at the percentage identity selected. The following table shows exemplary conservative amino acid substitutions.

TABLE 1
Exemplary conservative amino acid substitutions listed

| | Very Highly - | Highly Conserved | |
|---|---|---|---|
| Original | Conserved | Substitutions (from the | Conserved Substitutions |
| Residue | Substitutions | Blosum90 Matrix) | (from the Blosum65 Matrix) |
| Ala | Ser | Gly, Ser, Thr | Cys, Gly, Ser, Thr, Val |
| Arg | Lys | Gln, His, Lys | Asn, Gln, Glu, His, Lys |
| Asn | Gln; His | Asp, Gln, His, Lys, Ser, | Arg, Asp, Gln, Glu, His, Lys, |
| | | Thr | Ser, Thr |
| Asp | Glu | Asn, Glu | Asn, Gln, Glu, Ser |
| Cys | Ser | None | Ala |
| Gln | Asn | Arg, Asn, Glu, His, Lys, | Arg, Asn, Asp, Glu, His, Lys, |
| | | Met | Met, Ser |
| Glu | Asp | Asp, Gln, Lys | Arg, Asn, Asp, Gln, His, Lys, |
| | | | Ser |
| Gly | Pro | Ala | Ala, Ser |
| His | Asn; Gln | Arg, Asn, Gln, Tyr | Arg, Asn, Gln, Glu, Tyr |
| Ile | Leu; Val | Leu, Met, Val | Leu, Met, Phe, Val |
| Leu | Ile; Val | Ile, Met, Phe, Val | Ile, Met, Phe, Val |
| Lys | Arg; Gln; Glu | Arg, Asn, Gln, Glu | Arg, Asn, Gln, Glu, Ser, |
| Met | Leu; Ile | Gln, Ile, Leu, Val | Gln, Ile, Leu, Phe, Val |
| Phe | Met; Leu; Tyr | Leu, Trp, Tyr | Ile, Leu, Met, Trp, Tyr |
| Ser | Thr | Ala, Asn, Thr | Ala, Asn, Asp, Gln, Glu, Gly, |
| | | | Lys, Thr |
| Thr | Ser | Ala, Asn, Ser | Ala, Asn, Ser, Val |
| Trp | Tyr | Phe, Tyr | Phe, Tyr |
| Tyr | Trp; Phe | His, Phe, Trp | His, Phe, Trp |
| Val | Ile; Leu | Ile, Leu, Met | Ala, Ile, Leu, Met, Thr |

In some examples, variants can have no more than 3, 5, 10, 15, 20, 25, 30, 40, 50, or 100 conservative amino acid changes (such as very highly conserved or highly conserved amino acid substitutions). In other examples, one or several hydrophobic residues (such as Leu, Ile, Val, Met, Phe, or Trp) in a variant sequence can be replaced with a different hydrophobic residue (such as Leu, Ile, Val, Met, Phe, or Trp) to create a variant functionality similar to the disclosed an amino acid sequences of interest, such as SEQ ID NOs:1-12.

In some embodiments, variants may differ from the disclosed sequences by alteration of the coding region to fit the codon usage bias of the particular organism into which the molecule is to be introduced. In other embodiments, the coding region may be altered by taking advantage of the degeneracy of the genetic code to alter the coding sequence such that, while the nucleotide sequence is substantiality altered, it nevertheless encodes a protein having an amino acid sequence substantially similar to the disclosed an amino acid sequences of interest, such as SEQ ID NOs:1-12.

In some embodiments, functional fragments derived from SEQ ID NOs:1-12 of the present disclosure are provided. In some embodiments, the functional fragments share at least 80%, 85%, 90%, 95%, 96%, 97%, 98%, 99%, or more identity to SEQ ID NOs: 1-12 of the present disclosure.

The present disclosure provides agricultural compositions, combinations, processes, systems, and kits comprising the small molecules and their use for regulating ethylene signaling in a plant or plant tissue culture.

In some embodiments of the present disclosure, the agricultural compositions, combinations, processes, systems and kits involve using a dry granular formulation or a liquid formulation comprising the small molecules.

In some embodiments of the present disclosure, the agricultural compositions, combinations, processes, systems, and kits comprise using an adjuvant combined with the small molecules.

In some embodiments of the present disclosure, the agricultural compositions, combinations, processes, systems and kits involve combining the small molecules with or sequentially or simultaneously administering the small molecules with additional micronutrients, macronutrients, synthetic herbicides, different biological compounds, and/or inorganic compounds.

In some embodiments of the present disclosure, the agricultural compositions, combinations, processes, systems, and kits involve using seed coatings or seed inoculants comprising the small molecules.

In some embodiments of the present disclosure, the agricultural compositions, combinations, processes, systems, and kits comprise administering or applying the small molecules at an effective rate to regulate ethylene signaling in a plant or a plant tissue culture.

In some embodiments of the present disclosure, the agricultural compositions, combinations, processes, systems, and kits comprise administering or applying the small molecules at an effective rate to cause a change in the root system architecture of a plant or a plant tissue culture.

In one embodiment of any one of the methods disclosed herein, the small molecule(s) and agricultural composition(s) are applied simultaneously or sequentially.

### II. Root Systems of Plants

The root systems of plants play a crucial role for plant survival and productivity as they are the key organs to capture water and nutrients from the soil. For efficiently foraging the soil for nutrients, the distribution of roots, root system architecture (RSA), is of enormous relevance. Consequently, to sustain and improve crop productivity in the light of a changing climate, RSA and specific changes to it will be of key relevance (Lynch, 2021). For instance, to enhance the capacity for root systems to efficiently uptake nitrogen and water, deeper roots are required, yet for improving the uptake of phosphate and other nutrients shallower soil layers have to be accessed by roots (Lynch, 2019). Deeper rooting is also associated with higher carbon permanence (Poirier et al., 2018), a key trait for soil carbon sequestration and the ability of plants to survive terminal droughts (Uga et al., 2013). A key determinant of RSA is the lateral root angle or gravitropic set-point angle (GSA). Previous studies have shown that auxin plays a critical role in regulating GSA (Roychoudhry et al., 2013; Rosquete et al., 2013; Xiao et al., 2020). Application of exogenous IAA or synthetic auxin leads to lateral root growth toward the vector of gravity thereby decreasing GSA (Roychoudhry et al., 2013; Rosquete et al., 2013). Another phytohormone, cytokinin, was also found to be involved in the regulation of GSA. Application of cytokinin showed an anti-gravitropic effect and thereby increased GSA (Waidmann, 2019).

Ethylene is an ancient plant hormone whose signaling pathway is highly conserved and which has evolved in plants over 450 million years ago (Ju, 2015). Ethylene is produced from the conversion of S-adenosyl-L-methionine (SAM) to 1-aminocy-clopropane-1-carboxylic acid (ACC) and then to ethylene, a step which is catalyzed by enzymes including ACC synthase (ACS) and ACC oxidase (ACO) (Yang & Hoffman, 1984; Pattyn et al., 2021). Ethylene or ACC treatment induce a specific phenotype, coined the triple response, which includes shortened and thickened roots and hypocotyls, and an exaggerated apical hook (Guzman, 1990). Based on the triple response, previous studies have elucidated a comprehensive model of the ethylene signaling pathway in Arabidopsis thaliana (Arabidopsis) (Guo & Ecker, 2004). When ethylene binds to its receptors (ETRs) (Chang et al., 1993; Sakai et al., 1998; Hua et al., 1998), it inactivates a serine/threonine protein kinase, CONSTITUTIVE TRIPLE RESPONSE1 (CTR1), which is the most upstream negative regulator of ethylene signaling (Kieber et al., 1993). In the absence of ethylene, CTR1 blocks ethylene downstream responses by phosphorylating the C-terminal of ETHYLENE-INSENSITIVE2 (EIN2), an ER-associated membrane protein that works as a positive regulator of ethylene signaling (Alonso et al., 1999; Ju et al., 2012; Wen et al., 2012). This leads to the degradation of EIN2-CEND by the Ub/26S proteasome (Ju et al., 2012; Wen et al., 2012; Qiao et al., 2012). When CTR1 is inactivated by ethylene binding to ETRs, EIN2-CEND is stabilized and moves into the nucleus to transduce the ethylene signal to downstream transcription factors, such as EIN3, EIN3 LIKE1 (EIL1) (Chao et al., 1997; An et al., 2010) and ETHYLENE RESPONSE FACTORs (ERFs) (Muller & Munne-Bosch, 2015). These transcription factors activate the downstream ethylene response. The ethylene pathway was found to play an important role in numerous of growth and developmental processes in the root, such as the inhibition of root elongation (Kieber et al., 1993; Vaseva et al., 2018; Le et al., 2001), induction of root hair growth (Feng et al., 2017), and inhibition the initiation of lateral roots (LRs) (Negi et al., 2008).

### III. Benzimidazole Compounds

Mebendazole (MBZ; PubChem CID 4030; C₁₆H₁₃N₃O₃), also known as Vermox, Telmin and Mebenvet, has a molecular weight of 295.29 g/mol. Mebendazole is a white to slightly yellow powder, has a pleasant taste, and is practically water insoluble.

Mebendazole is an anthelmintic agent used commonly for roundworm (pinworm and hookworm) infections, trichinosis, capillariasis and toxocariasis and other parasitic worm infections. Mebendazole when given for prolonged periods in high doses has been associated with elevations in serum enzyme levels, and rare instances of acute, clinically apparent liver injury have been linked to its use.

Mebendazole is a synthetic benzimidazole derivate and anthelmintic agent. Mebendazole interferes with the reproduction and survival of helminths by inhibiting the formation of their cytoplasmic microtubules, thereby selectively and irreversibly blocking glucose uptake. This results in a depletion of glycogen stores and leads to reduced formation of ATP required for survival and reproduction of the helminth. This eventually causes the helminths death.

Deok-Soo Son et al. (The Antitumor Potentials of Benzimidazole Anthelmintics as Repurposing Drugs, Immune Network, 2020 August:20(4):e29, 20 pages, pISSN 1598-2629.eISSN 2092-6685) summarizes the central literature regarding the anticancer effects of benzimidazole anthelmintics, including MBZ, in cancer cell lines, animal tumor models, and clinical trials.

The present disclosure includes the use of benzimidazole compounds to activate the ethylene signaling pathways in plants, plant cells, plant tissues and plant parts. Examples of suitable benzimidazole drugs include but are not limited to albendazole, mebendazole, flubendazole, fenbendazole, and the derivatives thereof, analogues thereof, and isoforms thereof. The present disclosure includes the use of mebendazole and derivatives, analogues and isoforms of mebendazole which activate the ethylene signaling pathway in plants, plant cells, plant tissues and plant parts. Isoforms of mebendazole include but are not limited to mebendazole C1 (M-C1) and mebendazole C2 (M-C2). See, e.g., Xu et al., 2019, In vitro efficacies of solubility-improved mebendazole derivatives against Echinococcus multilocularis, Parasitology, Vol. 146, Issue 10. Derivatives of mebendazole include but are not limited to carbamate and acyloxymethyl derivatives of mebendazole. See, e.g., Studenovsky et al., 2021, Polymers, Vol. 13, Issue 15.

Chemical structure depiction of MBZ:

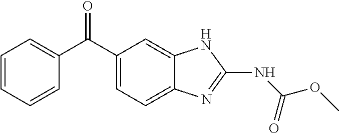 [SMILES: COC(=O)Nc1nc2ccc(C(=O)c3ccccc3)cc2[nH]1]

### IV. Application of Small Molecules

Various methods of application of small molecules are possible, and each may be preferred under certain circumstances. One method of application is in the form of a foliar spray. A solution comprising a small molecule (i.e., MBZ) is prepared. The concentration of the solution is determined by its uses, and the liquid solution sprayed onto the plants. A topical spray for herbs, crop plants, vegetables, or fruit trees with the solution comprising the small molecule(s) can be used.

In some embodiments, spray solutions contain a wetting agent.

An alternative method of application involves adding a liquid form of solution or a dry form of powder, each of which comprises a small molecule (i.e., MBZ) directly to the soil.

The liquid solution with small molecule(s) can be added to the soil as a drench, added to the soil near the root zone of plants or banded near the root zone of row crops (commercial crops, vines, trees).

Another method of application involves direct inoculation of plants with a solution of MBZ. It may also be applied as a powder to plants.

In some embodiments, a small molecule (i.e., MBZ) is applied to the plant by any convenient method, e.g., spraying or coating with a powder, emulsion, suspension, or solution.

In other embodiments, MBZ is applied to the soil by any convenient method, e, g., spraying a solution, emulsion, or suspension or applying a powder.

Additional method of application involves applying a small molecule to a growing medium used to grow the plant or the plant tissue culture.

In some embodiments, the applying is accomplished by spraying the small molecule onto the plant or plant tissue culture or the soil. The applying is accomplished by using a liquid comprising the small molecule or a dry form of powder comprising the small molecule.

### Examples

The present disclosure is further illustrated by the following examples that should not be construed as limiting. The contents of all references, patents, and published patent applications cited throughout this application, as well as the Figures, are incorporated herein by reference in their entirety for all purposes.

### Methods

Plant material and growth conditions. Arabidopsis mutants and transgenic lines used in this study are in the Col-0 background if not mentioned otherwise. etr1-1 (CS237) and etr1-3 (CS3070) were ordered from ABRC, ctr1-1 (Kieber, 1993) ein2-5 (Alonso, 1999), ein3-1eil1-1 (Alonso, 2003), ein3eil135S: EIN3-GFP (He, 2011) were lab stock.35S:EIN2-YFP (gift from Dr. Caren Chang, University of Maryland), UBQ10p: TUB6-GFP, (Nakamura et al., 2004) 35S:DR5v2:3×YFP-NLS (Liao, 2015) (Gift from Weijers lab, Col-Ultrecht background).

Arabidopsis thaliana seeds were sterilized as previous described (Li et al., 2019). Briefly, the seeds were sterilized using chlorine gas produced from the mixture of 200 ml 8.25% sodium hypochlorite (Bleach, Clorox) and 3.5 ml 37% hydrochloric acid in a sealed box for 1 h, and then were stratified in water at 4° C. for 3 days in dark. The seeds were sown on the 12 MS media (pH5.70), 0.1% MES, 1% Sucrose, 1% agar plates with chemicals or control (DMSO) and then these plates were vertically positioned in racks in a walk-in growth chamber in long day conditions (16/8 h) at 21° C., 50 uM light intensity, 60% humidity. During night time, temperature was decreased to 15° C.

Small Molecule Library and Screen Information. The SP2000 small molecule library was received from the University of California, Riverside. The library was screened at a concentration of 50˜100 μM in 200 μL of liquid MS media. After being grown in the liquid media for 7 days, seedlings were transferred to vertical plates (3 replicates for every chemical treatment) to grow for another 2 days. The root phenotypes were acquired by CCD flatbed scanners. After first round of screen, several candidates were picked. Then, a second round of screen by gradient concentration of candidate small molecules from 1 μM to 100 μM on vertical plates was performed to confirm the phenotype and optimal concentration.

Chemical Solutions. The stock solutions for preparing media for the plant experiments were Mebendazole (10 mM) in DMSO, ACC (10 mM) in dH2O, IAA (1 mM) in ethanol.

Root phenotyping. Root phenotype images on plates were acquired with CCD flatbed scanners (EPSON Perfection V600 Photo, Seiko Epson CO., Nagano, Japan). Root lengths and lateral root angles were measured using Fiji (http://fiji.sc/Fiji).

Time lapse of lateral root angle phenotyping. 14 d old seedling on DMSO plate or 19 d old seedling on mebendazole plate were transferred to DMSO or 1 μM mebendazole plates as described. Root phenotype images of these plates were acquired by CCD vertical scanners (EPSON Perfection V600 Photo, Seiko Epson CO., Nagano, Japan) every 10 mins over a period of 24 hours using the tool described (Ogura et al., 2022). Root length and lateral root angles were measured using Fiji.

mRNA-seq analysis. 14 d old seedlings on DMSO plates were transferred to 12 MS liquid plates with DMSO or 10 μM mebedazole treatment for 4 h, root tissues were collected for RNA extraction and RNA-seq.

Read alignment and generation of counts. The TAIR10 genome file and annotation file were obtained from the Arabidopsis Information resource web site (arabidopsis.org). The An aligner, called the Splice Transcripts Alignments to Reference (STAR) version 2.7.0a⁴⁴, was used to align short reads in the FASTQ files. In order to use STAR, a STAR index was built using the following parameters:

$ STAR --runThreadN 4 \
--runMode genomeGenerate \
--genomeDir ara_star_index \
--genomeFastaFiles <TAIR10.fa>\
--sjdbGTFfile <TAIR10.gtf> \
--sjdbOverhang 99

Then, short reads in the FASTQ files were aligned and gene counts were obtained using the following STAR command:

$ STAR --genomeDir ara_star_index \
--run ThreadN 8 \
--sjdbOverhang 99 \
--sjdbGTFfile <TAIR10.gtf> \
--outSAMtype BAM SortedByCoordinate \
--outFileNamePrefix /output/small_ \
--outReadsUnmapped Fastx \
--quantMode GeneCounts \
--readFilesIn <fastq>

A custom R script was used to combine counts per gene from count data produced from STAR cross all samples.

Differential expression analysis. Differential expression of genes between treatments and control was determined using the R package, edgeR (version 3.36.0) (Robinson et al., 2010). The CPM (counts per million) function from edgeR was used to normalize the counts and differentially expressed genes (DEGs) were identified using glmLRT function from edgeR. A false discovery rate (FDR<0.05) and |log 2FC|>1 were used as the criterial values for identification of up-regulated and down-regulated DEGs.

Analysis of published Ethylene RNA-Seq. The fastq-dump utility from the Short Read Archive (SRA) Toolkit (v2.6.3) (github.com/ncbi/sra-tools) was used to download transcriptome libraries (SRA accession IDs: SRR648276, SRR648275, SRR648274, SRR648300, SRR648299, and SRR648298) from SRA database (ncbi.nlm.nih.gov/sra) (Leinonen et al., 2011). The color space sequencing data were converted to base-space sequencing data using Perl script ‘csfq2fq.pl’ (gist.github.com/pcantalupo/9c30709fe802c96ea2b3). The converted FASTQ files were aligned and analyzed using above methods.

Measurement of ethylene production. One hundred (100) seeds of Col-0 were planted and sealed in a screw-top 20 mL vial containing 10 mL 12 MS medium supplemented with DMSO (control), 10 μM mebendazole, and 10 μM ACC treatments (4 replicates). These vials were incubated at 22° C. for 3 days under dark environment. 500-1000 μL sample of the headspace gas was sampled with an autosampler (TriPlus RSH, Thermo Scientific) and injected into a gas chromatograph (Trace 1310 GC, Thermo Scientific) that was equipped with a HP-PLOTQ column (30 mm, 320 μm, 20 μm) and a mass spectrometer (TSQ8000 Evo MS, Thermo Scientific), scanning from 25-27.5 m/z. Separations were carried out at 35° C. using He as the carrier gas. The area of the ethylene peak (RT: 4 min) was integrated using Thermo Xcalibur Qual Browser. A calibration curve was generated by varying the injection volume (100 μL, 250 μL, 500 μL, and 1000 μL) of a 10 ppm ethylene standard (in nitrogen), and sample results are expressed as concentrations calculated from linear regression of calibration samples.

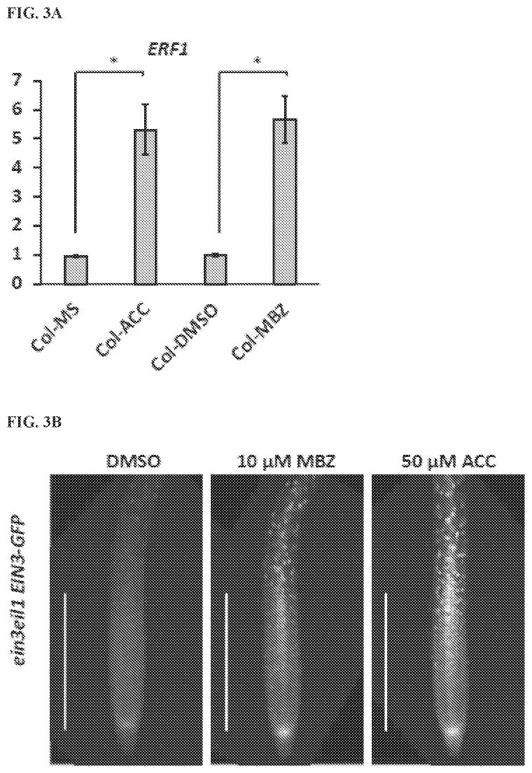

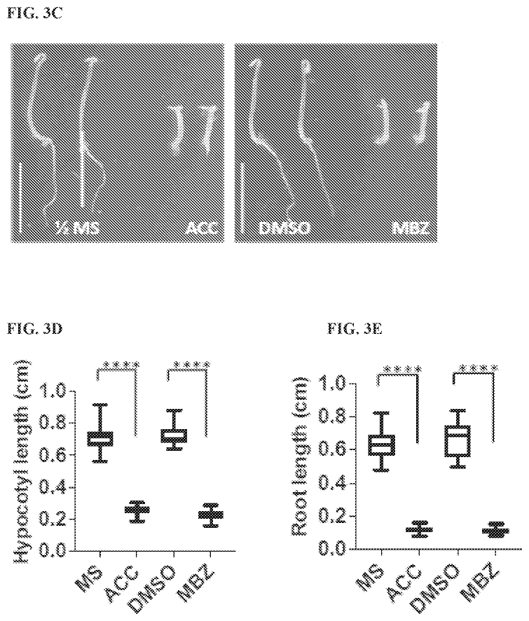

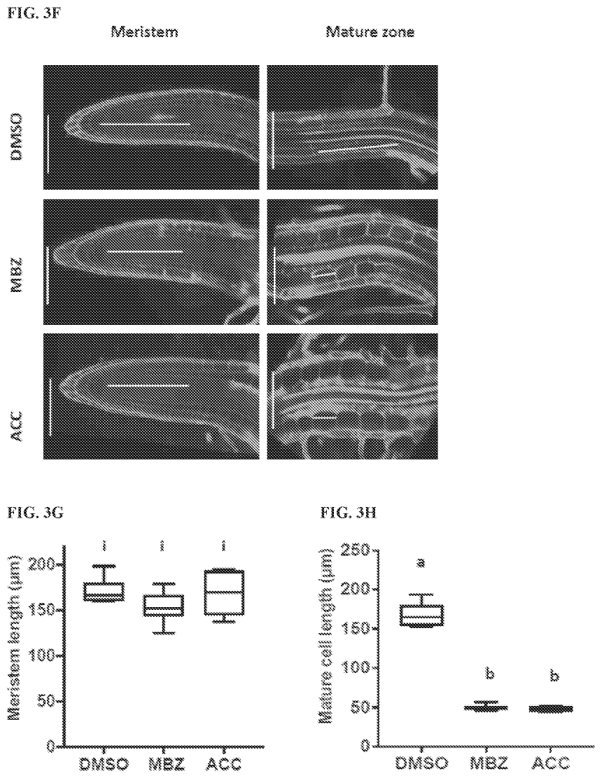

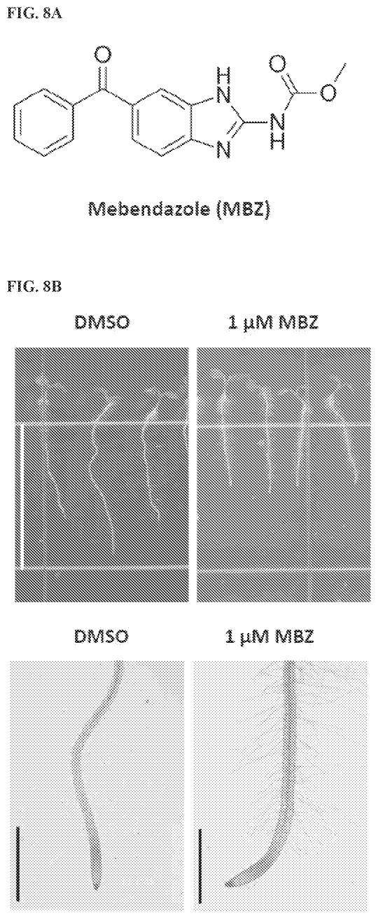

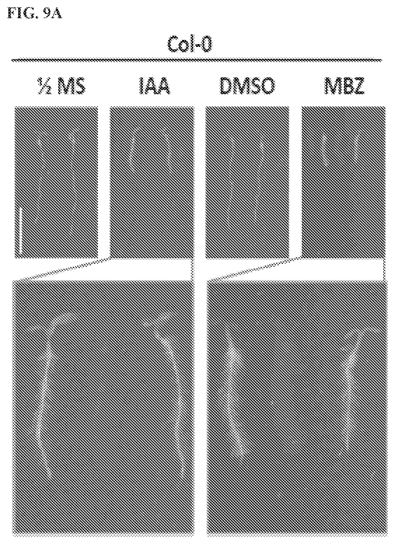

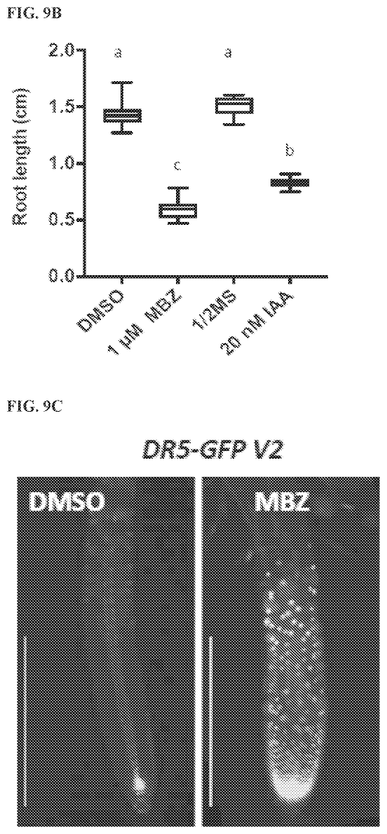

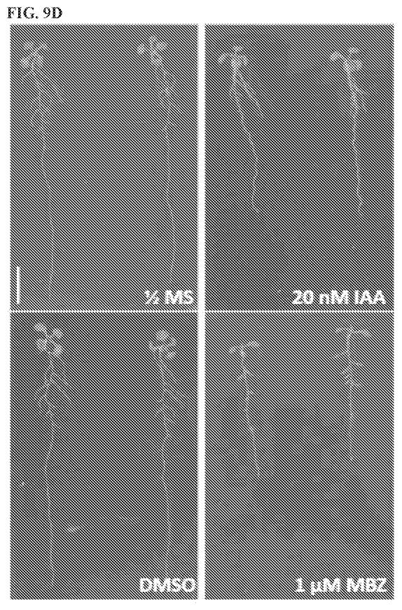

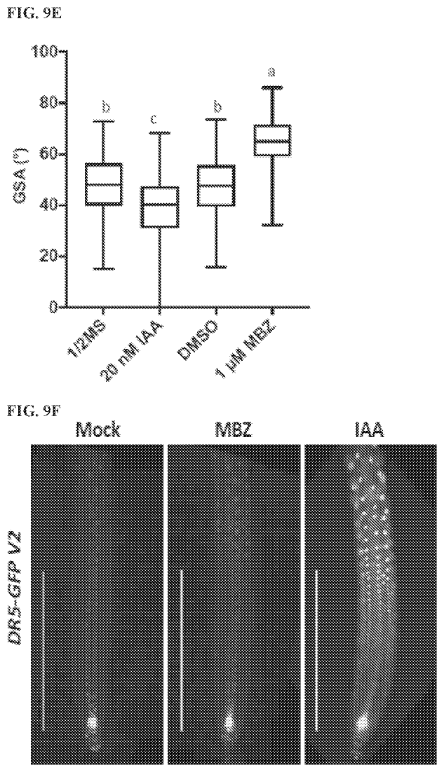

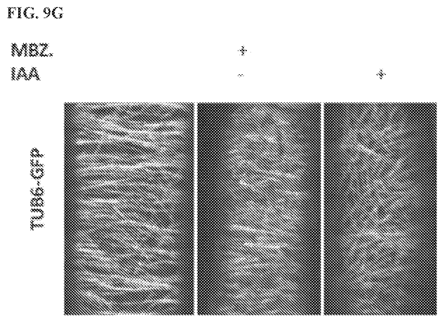

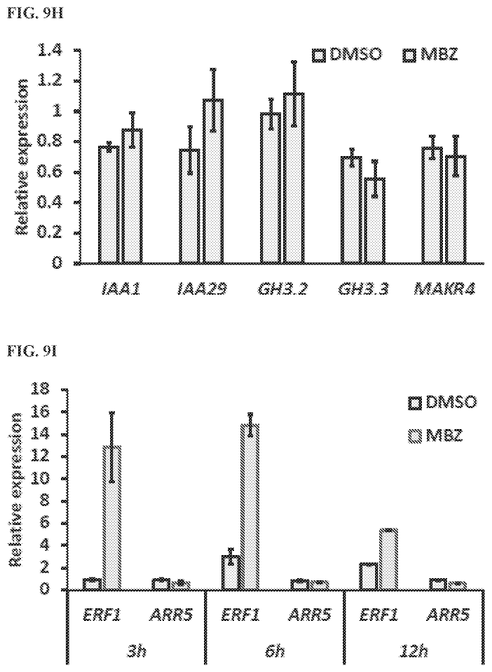

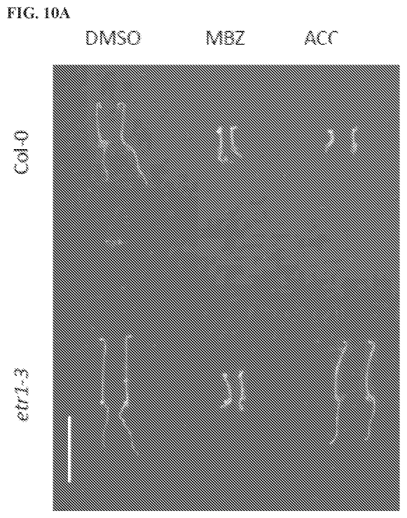

LaserMicroscopy. A Zeiss 710 confocal microscope with 20×, 40×, and 63× objectives were used to detect GFP and propidium iodide (PI) fluorescence. A Keyence BZ-X810 microscope with a 20× objective was used to detect bright field images and GFP fluorescence in FIG. 3B, FIG. 8B right panel, FIG. 9C, FIG. 9F.

Computational Docking and Molecular Modeling. The AutoDock Vina and Chimera software were used to dock mebendazole (CHEMBL685) into crystal structure of CTR1 (PDB: 3PPZ) and MAPK14 (PDB: 6SFO). Figures for those structures were visualized and generated by Chimera and Pymol.

Protein purification. The CTR1 Kinase domain (540 aa-821 aa) (CTR1-KD) and MAPK4 were cloned into a modified pET28a with 6× His tag at their N-terminal. Proteins were expressed in Rosetta™ 2 (DE3)pLysS Competent Cells. Bacteria was cultured in 30 mL LB media containing 50 μg/mL kanamycin overnight at 37° C., 200 rpm. The culture was transferred into 1 L LB media with kanamycin. After the culture reached to 0.7 to 0.9 at OD 600 nm, 0.4 mM isopropylthiop-D-galactoside (IPTG) was added, and the culture was grown for 22 h at 20° C., 200 rpm. Cells were harvested by centrifugation at 4° C. for 10 mins at 3,000×g. The pellet was washed with 0.9% NaCl and resuspended in 40 mL lysis buffer (50 mM pH 7.5 Tris-HCl, 1 mM EDTA, 100 mM NaCl, 20% glycerol, EDTA-free protease inhibitor cocktail, 10 mM imidazole). Following resuspension, cells were sonicated for 1 min 40 sec (for 2 rounds) in 50 mL beaker sitting in ice water mixture bath-2 seconds on/2 seconds off (Amplitude 50%). The cell lysate was clarified at 13,000×g 20 min at 4° C. The supernatant was filtered with 0.8 μm Syringe filter (Acrodisc) and first purified using QIAGEN Ni-NTA Agarose. The elute from Ni-NTA was collected and further purified by FPLC using superdex 200 Increase 10/300 column (Berardini et al., 2015). The fractions corresponding to single peak from FPLC were combined, and concentrated to a desired volume. Protein purity was verified by Coomassie blue gel staining. Protein concentration was measured by their absorbance at 280 nm.

In vitro kinase assay with non-radioactive ATP-7-S. The in vitro kinase assay followed the previous published method (Dobin et al., 2013). 100 ng purified recombinant CTR1-KD or MPK4 was mixed with 2 μg Myelin Basic Protein (MBP, a common kinase substrate, Sigma Cat. M1891) and 1 mM ATPyS in kinase reaction buffer (10 mM HEPES pH 7.4, 150 mM NaCl, 10 mM MgCl2, 1×Roche Comlete Protease Inhibitor mixture) and different concentration of MBZ at room temperature and vortexed for 1 hour. The reactions were terminated by 20 mM EDTA, and the proteins were alkylated by adding 1.5 uL of 50 mM PNBM (p-Nitrobenzyl mesylate from CAYMAN CHEMICAL CO, Cat. 21456-1) at room temperature and vertexed for 2 hours. Then NuPAGE™ LDS Sample Buffer (Invitrogen™ Cat. NP0008) supplemented with 1× NuPAGE™ Sample Reducing Agent (Invitrogen™, Cat. NP0009) was added. The protein samples were denatured for 10 minutes at 90° C. and then centrifuged at 13,000 rpm for 10 minutes. The supernatant protein samples were separated by NUPAGE 10% Bis-Tris Plus Gel (Invitrogen™, Cat. NW00105BOX) and transferred onto Nitrocellulose membrane by the iBlot 2 Dry Blotting system (Invitrogen™, Cat. 1323001). The phosphorylation status of MBP, CTR1-KD and MPK4 were analyzed by western blot using Recombinant Anti-Thiophosphate ester antibody (Abcam, Cat. Ab92570, 1:5000) followed by Goat Anti-Rabbit IgG (H+L)-HRP Conjugate antibody (Bio-Rad, Cat. No. 170-6515, 1:5000). The MBP, CTR1-KD and MPK4 proteins loading amounts were measured by Coomassie Blue R250 staining.

Statistical analysis. A two-sided Student's t-test was used for comparisons of two conditions. The comparison of meristem length, mature cell length, root length, GSA between multiple treatments or genotypes was performed by One-way ANOVA and post hoc Tukey testing. Detailed information was prepared in figure legends. Statistical significance of overlap between DEGs from MBZ treatment and ethylene treatment was assessed by hypergeometric distribution test. Hypergeometric test function in R was used to calculate statistical significance:
$ phyper(q−1, m, n-m, k, lower.tail=FALSE, log.p=FALSE)
q=the number of genes in common between two sets
m=the number of genes in Set 1;
n=the total number of genes in RNA-Seq counts table (33,602)
k=the number of genes in Set 2

### Example 1: A Small Compound Profoundly Affects Root System Architecture in Arabidopsis

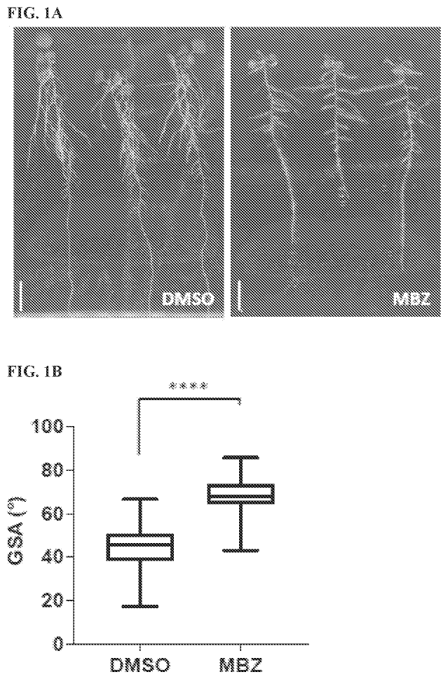

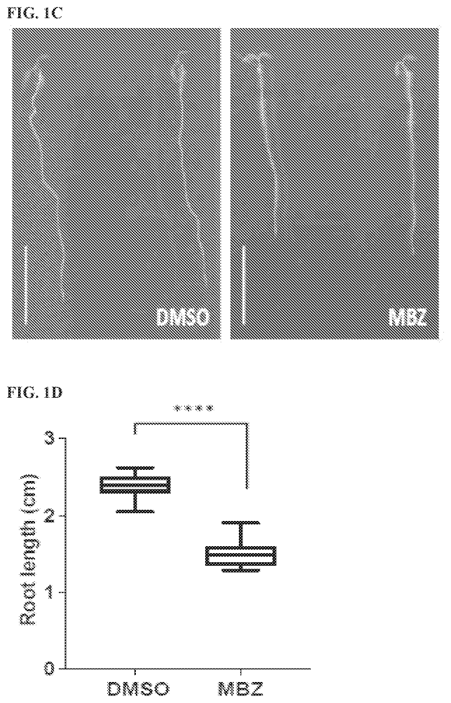

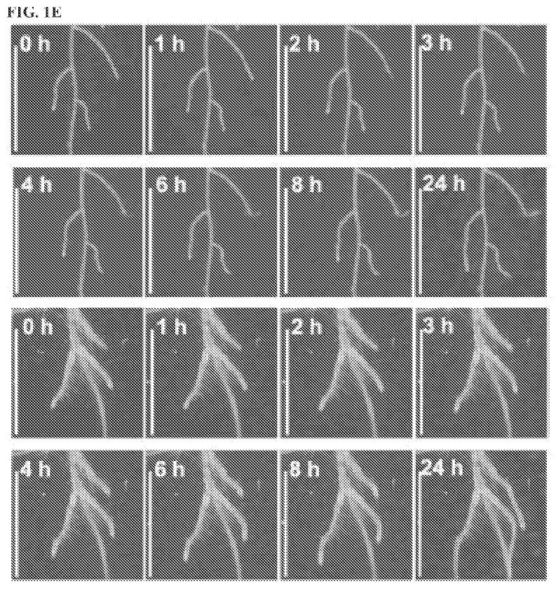

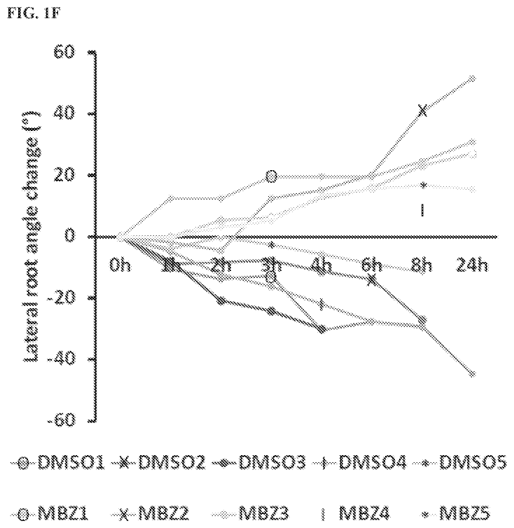

To identify small molecules that affect RSA, we screened chemicals for their ability to change RSA of young seedlings from a small molecular library with 2000 diverse generic chemicals (SP 2000) (He et al., 2011; Sun et al., 2017; Zhu et al., 2019). We found that treatment with Mebendazole (MBZ) (FIG. 8A) led to a distinct increase of lateral root angles that caused lateral roots to grow in a more horizontal direction (FIG. 1A). The lateral root gravitropic setpoint angle (GSA) of untreated two-week-old Arabidopsis seedlings was approx. 46°, and the GSA of lateral roots of seedlings grown on 1 μM MBZ plates was approx. 70° (FIG. 1A, FIG. 1B). While the lateral root angle was the most striking phenotype, at a much earlier developmental stage (one week after germination), seedlings on 1 μM MBZ displayed a decreased root length (in average reduced by 37%) and an increased root hair initiation and elongation (FIG. 1C, FIG. 1D, FIG. 8B).

To test how quickly MBZ would affect root growth, we grew seedlings for 14 days on 12 MS medium with DMSO, then transferred them to 12 MS medium supplemented with 1 μM MBZ and acquired images every 10 minutes. All of the five measured lateral root tips started to grow at a more horizontal (less steep) angle within three hours of MBZ treatment (FIG. 1E upper panel, FIG. 1F).

To test whether the action of MBZ was reversible, we germinated and grew seedlings on 2 MS for 5 days, and then transferred them to medium containing 1 μM MBZ for 12 days, thereby inducing lateral roots of MBZ treated seedlings to grow more horizontal. Finally, we transferred the seedlings back to control conditions and acquired images every 10 minutes. All 5 lateral root tips we measured started to grow at a distinctly steeper angle within four hours upon removal from MBZ (FIG. 1E bottom panel, FIG. 1F). These data showed that the treatment of Arabidopsis roots with MBZ changes lateral root angles within three hours and that this effect is fully reversible. In summary, we found that MBZ changes lateral root angles, and important RSA traits associated with the development of a shallow root system.

### Example 2: MBZ Treatment Specifically Impacts the Ethylene Pathway

Plant hormone pathways play a major role in determining RSA traits (Ristova et al., 2018; Ogura et al., 2019) and two plant hormones, auxin and cytokinin, have been shown to be involved in setting the GSA of lateral roots (Roychoudhry et al., 2013; Rosquete et al., 2013; Waidmann et al., 2019). To unravel the mode of action of MBZ impact, we therefore first tested whether MBZ specifically acts on auxin or cytokinin. We compared root phenotypes of 8-day-old Arabidopsis seedlings grown on media plates with 1 μM MBZ and 10 nM IAA. We found that both treatments inhibited root elongation and enhanced root hair growth (FIG. 9A, FIG. 9B). In addition, MBZ treatment induced DR5-GFP V2 expression in 7-day-old roots (FIG. 9C), suggesting auxin responses were induced by MBZ treatment. However, our data had shown that MBZ treatment distinctly increased lateral root angle, which is opposite to previous described auxin treated plants that showed a decrease in lateral root angle (Roychoudhry et al., 2013; Rosquete et al., 2013) and that could be reconfirmed under our conditions (FIG. 9D, FIG. 9E). As auxin signaling can be regulated downstream of other signaling pathways, we looked at the timing of the onset of the MBZ triggered auxin responses.

Based on our time-lapse experiment we could see a change of GSA within one to four hours of MBZ treatment (FIG. 1E, FIG. 1F), we assumed that any relevant direct response to MBZ should be observable within this timeframe. However, no increased DR5-GFP V2³⁴ expression in roots treated with MBZ was observed within 3 hours compared to mock treatment (FIG. 9F). To further exclude an involvement of the auxin response in the immediate response to MBZ, we measured changes in the orientation of microtubules occur, which is a hallmark response to auxin treatment (Chen et al., 2014). The orientation of P-tubulin in roots significantly changed under 1 μM IAA but not upon 50 μM MBZ within 1 h (FIG. 9G). Consistent with the results obtained from the DR5-GFP V2 reporter and the microtubule reorientation experiments, expression of all marker genes of auxin signaling that we checked, including IAA1, IAA29, GH3.2, GH3.3, and MAKR4 did not show any significant change upon MBZ treatment for 2 h (FIG. 911). Together these data strongly suggested that the increased DR5-GFP V2 signal under long-term treatment is caused by an indirect regulation of auxin signaling or a feedback effect. We therefore concluded that MBZ was not directly acting upon the auxin pathway.

We also tested whether the cytokinin response was elicited by MBZ treatment. For this, we first measured the expression of ARR5, which is one of the immediate-early cytokinin target genes. ARR5 expression did not respond to MBZ treatment (FIG. 9I) at 3 h, 6 h or 12 hours, suggesting that MBZ does not act upon the cytokinin pathway in the timeframe that we observe responses.

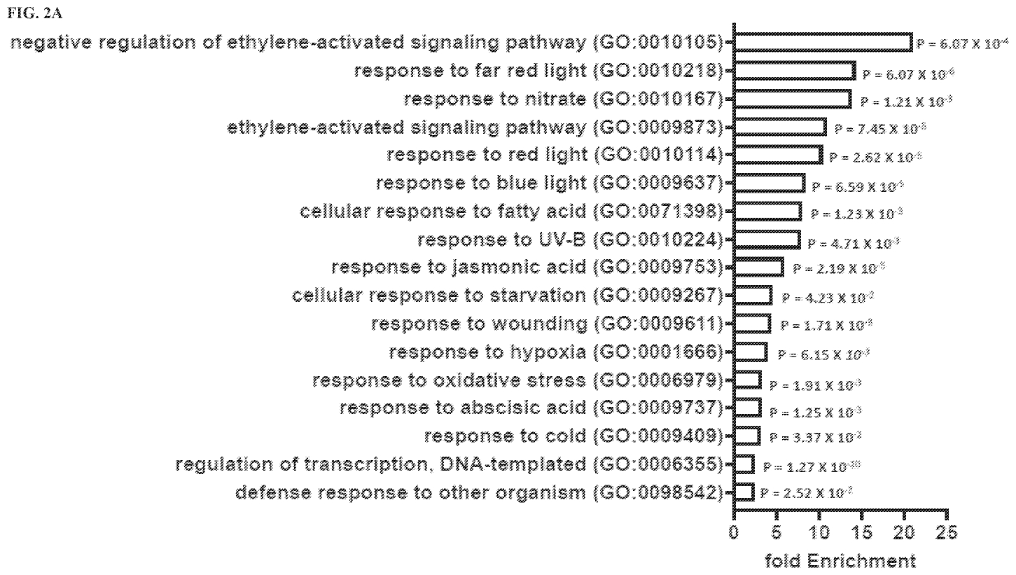

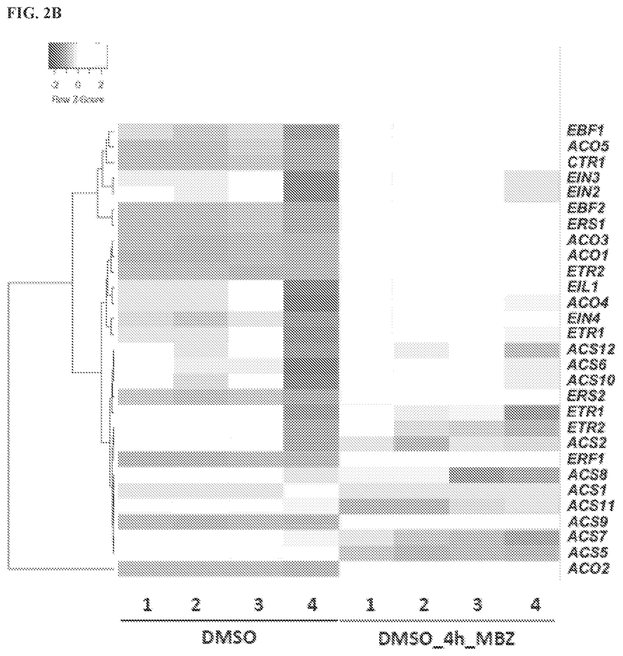

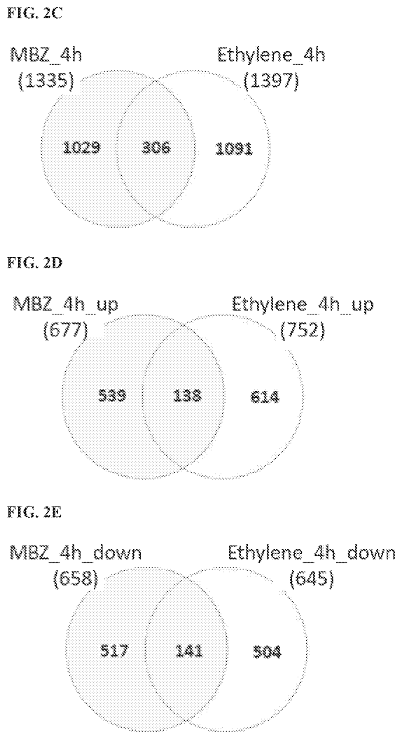

As we had not found evidence supporting the direct involvement of auxin and cytokinin in the immediate response to MBZ, we utilized a genome-scale expression profiling approach. For this, we performed an mRNA-seq experiment using roots of 14-days-old Arabidopsis grown on ½ MS media treated with 10 μM MBZ for 4 hours. The analysis revealed that 677 genes were induced compared to mock treatment (log 2 fold change >1) and 658 genes were repressed (log 2 fold change <−1). A gene ontology (GO) analysis revealed that among the genes that MBZ impacted, genes involved in ethylene responses were strongly overrepresented. In particular, the GO process “negative regulation of ethylene-activated signaling pathway” (GO:0010105) was the most enriched process (21.03 fold enrichment, P-value: 6.07E−04) upon MBZ treatment (FIG. 2A), and the 4^{th} most enriched GO process was “ethylene-activated signaling pathway” (GO:0009873) (10.83 fold enrichment, P-value:7.45E−05) (FIG. 2A).

To explore this in more detail, we directly looked at the responses of genes involved in the ethylene hormone pathway. A major portion of these genes were differentially regulated by MBZ compared to mock treatment (FIG. 2A, FIG. 2B). To further investigate this in a gene annotation independent manner, we compared the set of genes that had responded to MBZ with published data of 4 h ethylene treatment (Chang et al., 2013). Of the 1335 differentially expressed genes (DEGs) upon the 4 hours of MBZ treatment, 306 were overlapping with 1397 DEGs upon 4 hours of ethylene treatment (P-value: p<4.657e−143)(FIG. 2C). More than 90% of these common DEGs retained the directionality of their expression change (i.e. genes upregulated by MBZ were also upregulated by ethylene treatment and vice versa) (FIG. 2D, FIG. 2E). Overall, these data indicated that MBZ treatment acted on the ethylene pathway.

### Example 3: MBZ Treatment Mimics Effects of the Ethylene Precursor ACC to Induce Ethylene Responses

To investigate how MBZ treatment acts upon the ethylene response, we compared plant responses to MBZ and the ethylene precursor ACC. We focused on early responses within a two-hour timeframe, as we had observed the first changes in GSA of lateral roots to occur within three hours (FIG. 1F). The transcript level of ERF, a marker gene for ethylene responses, was strongly induced by 10 μM MBZ as well as ACC treatment in root tissue after treatment for 2 hours (FIG. 3A). Moreover, EIN3, a major transcription factor involved in ethylene signaling, is also dramatically induced by both treatments according to the 35S-EIN3-GFP protein level in ein3eil1 background after 2 hours (FIG. 3B). These results suggested that MBZ has a similar activity to ACC for triggering ethylene responses. We therefore reasoned that if MBZ induced similar effects to ACC, we should observe similar growth regulatory responses.

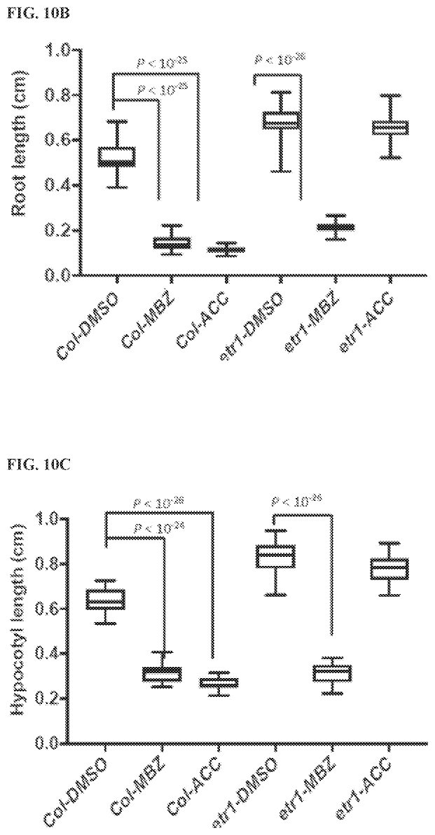

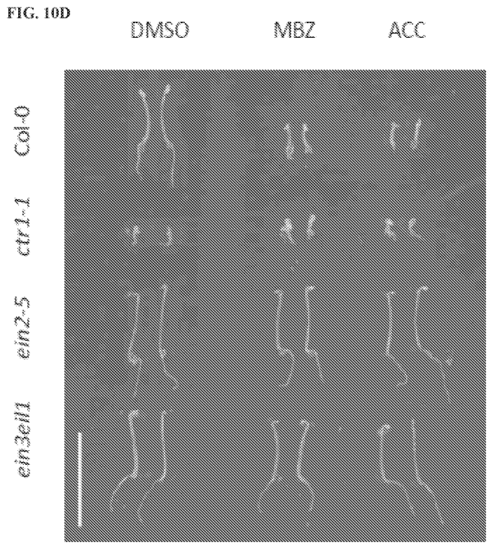

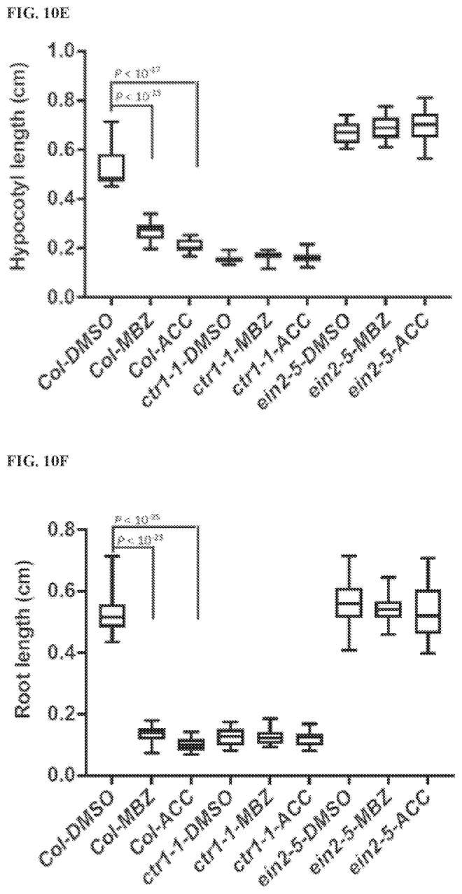

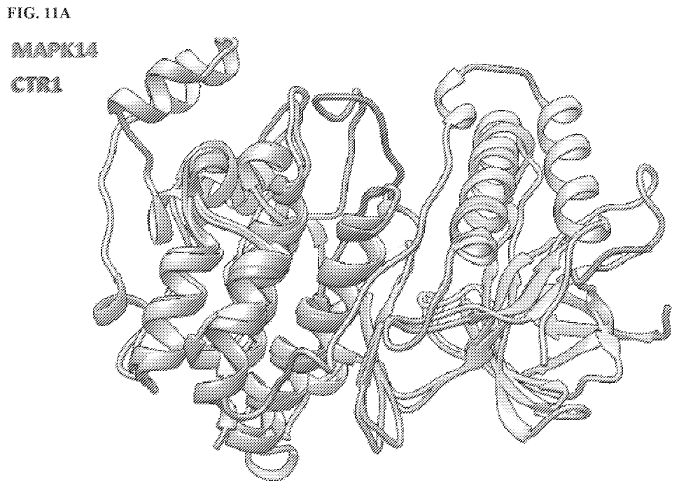

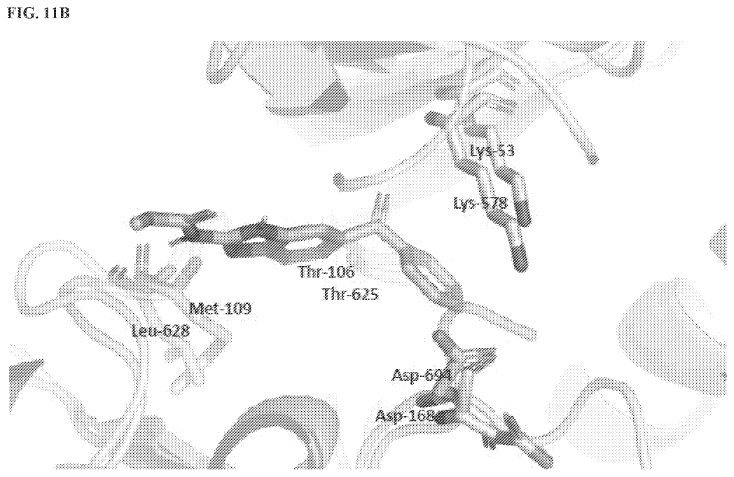

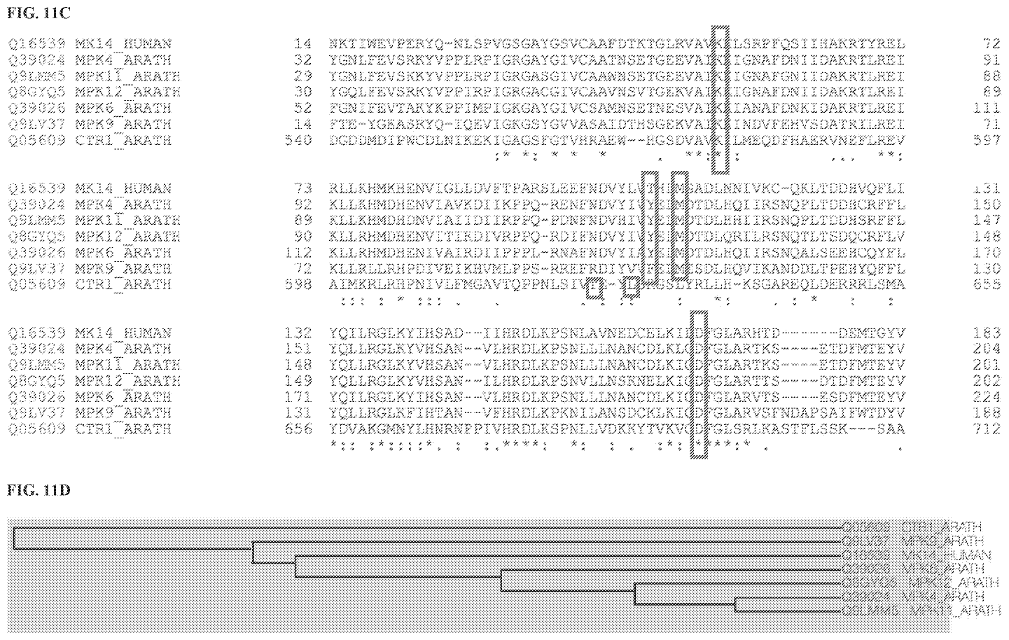

The most characteristic response to ACC and ethylene treatment is the triple response (Guzman, 1990). Consistent with our hypothesis, 3-day-old etiolated seedlings treated with 10 μM MBZ showed a significant triple response phenotype (FIG. 3C to FIG. 3E). Using different doses of MBZ, we found that 2.5 μM of MBZ was sufficient to induce the triple response phenotype in a dark environment (FIG. 10A to FIG. 10F). Consistent with MBZ mimicking ethylene treatment, this MBZ induced triple response was abolished in the ethylene insensitive mutants ein2-5 and ein3eil1 (FIG. 10D to FIG. 10F).

Ethylene treatment inhibits root elongation mainly by repressing cell elongation (Vaseva, 2018; De Cnodder et al., 2005). We therefore measured the length of root cells of 4-day-old etiolated seedlings treated with 2.5 μM MBZ and 10 μM ACC, and found that mature cell size was reduced by both treatments (FIG. 3F to FIG. 311). Altogether, our data strongly suggested that MBZ elicits its effects via the ethylene pathway.

### Example 4: MBZ Targets Ethylene Signaling

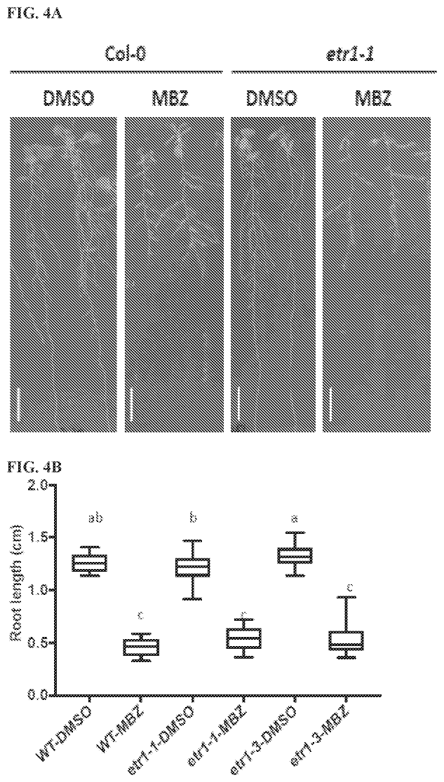

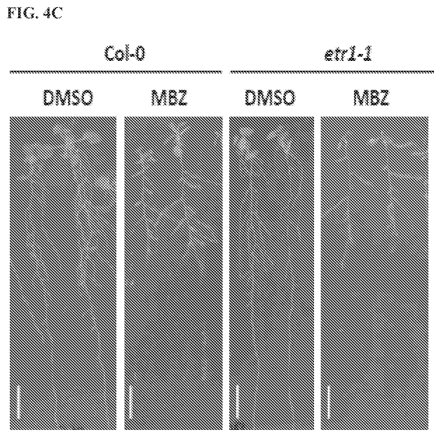

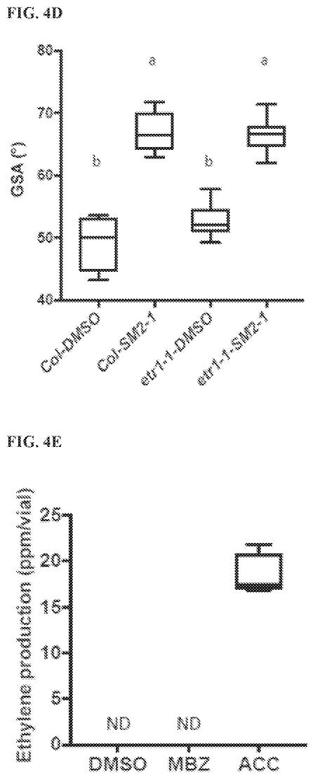

Next, we set out to locate the target of MBZ in the ethylene pathway. First, we tested whether MBZ induces ethylene biosynthesis, or acts on the perception of ethylene, or on the ethylene signaling pathway. To test whether biosynthesis or perception of ethylene is affected, we utilized the etr1 ethylene receptor mutants. Both strong alleles of etr1, etr1-1 and etr1-3, still strongly responded to MBZ treatment (FIG. 4A to FIG. 4D). Moreover, 4-days-old, etiolated seedlings of etr1-3 treated with 2.5 μM MBZ showed the typical triple response phenotype, demonstrating that ethylene responses are triggered by MBZ even without the perception of ethylene. In contrast to the response to MBZ, etr1-3 plants were completely insensitive to the treatment with 10 μM of ACC (FIG. 10A to FIG. 10C).

To further test the independence of MBZ action of ethylene biosynthesis, we measured ethylene production by GC-MS in 3-days-old, etiolated Col-0 seedlings that were treated with 10 μM MBZ or ACC. Consistent with our hypothesis, ethylene production was dramatically induced by 10 μM ACC, but not detected in DMSO or 10 μM MBZ treated seedlings (FIG. 4E). Collectively, these results show that MBZ induces ethylene responses by targeting factors downstream of ethylene biosynthesis and perception.

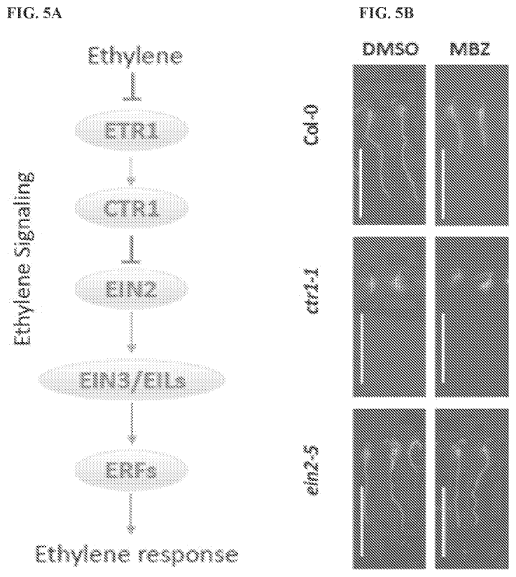

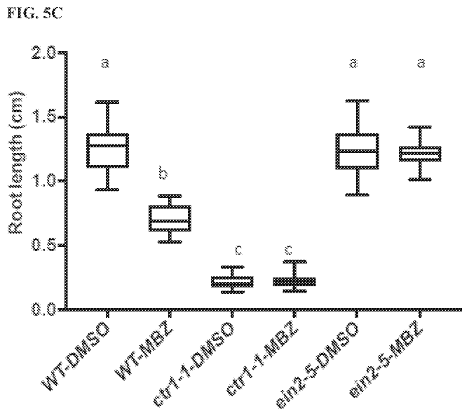

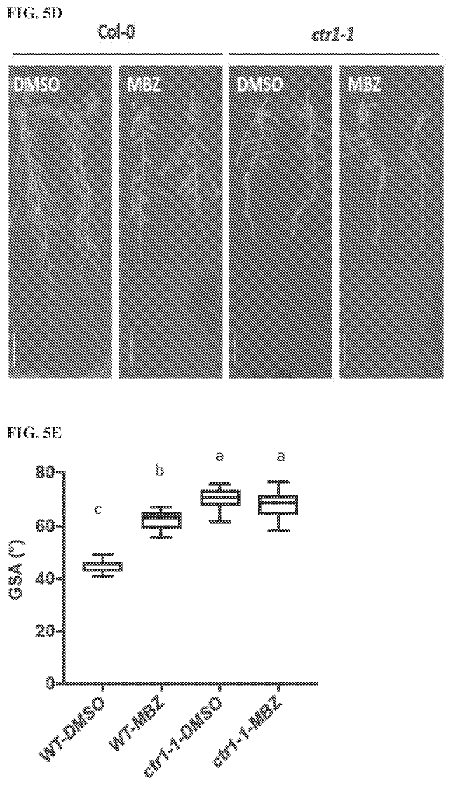

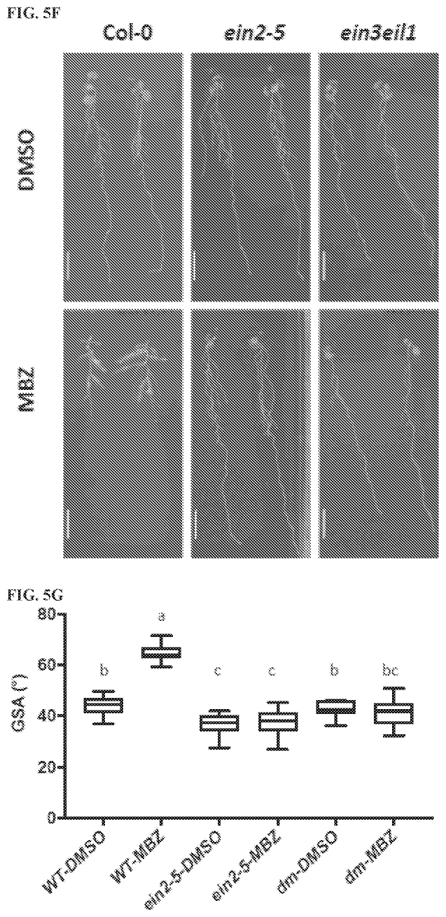

To uncover which components of ethylene signaling are targeted by MBZ, we studied MBZ responses in key mutants of the ethylene signaling pathway. First, we checked the root inhibition phenotype in ctr1-1, ein2-5, ein3eil1 mutants treated with MBZ and compared these responses to the Col-0 wildtype. CTR1 is the first component of ethylene signaling, and EIN2 acts downstream of CTR1 to trigger ethylene signaling. EIN3/EIL1 are core transcription factors of ethylene signaling, which work downstream of EIN2 (FIG. 5A). Roots of Col-0 were sensitive to 1.2 μM MBZ treatment and showed short roots and increased root hair development (FIG. 5B, FIG. 5C). However, ctr1-1 mutants in which ethylene signaling is constitutively active, showed short roots and abundant root hairs without any treatment at an early stage. Due to this pronounced early-seedling phenotype, we could not confidently assess changes in response to MBZ. The root length of ein2-5 and ein3eil1 mutant plants was clearly not affected by MBZ treatment (FIG. 5B, FIG. 5C).

We further measured lateral root angles at a later stage of development, which we had found to be a major effects of MBZ treatment. 2 to 3-weeks-old ctr1-1, ein2-5, and ein3eil1 plants were all insensitive to MBZ with regards to lateral root angles (FIG. 5D to FIG. 5G), suggesting that mutations in these genes interferes with the function of MBZ. Consistent with this hypothesis, ein2-5 and ein3eil1 didn't show a triple response upon MBZ treatment (FIG. 10D to FIG. 10F). ctr1-1 showed a constitutive triple response no matter whether it was treated with MBZ or not. Taken together our data showed that MBZ activates ethylene signaling by interfering with CTR1 or EIN2, two core factors in ethylene signaling pathway.

### Example 5: MBZ Inhibits CTR1 Kinase Activity

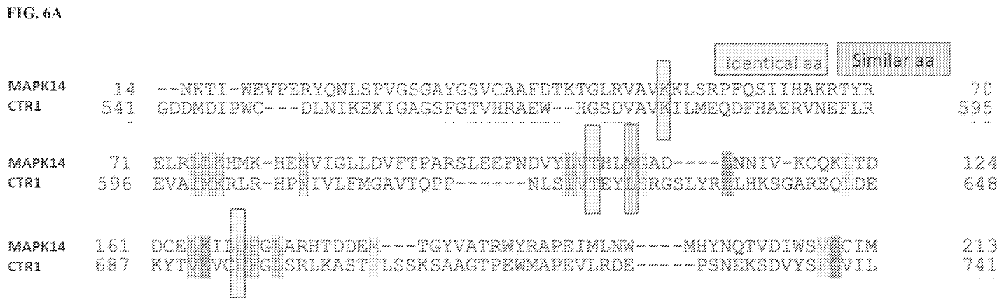

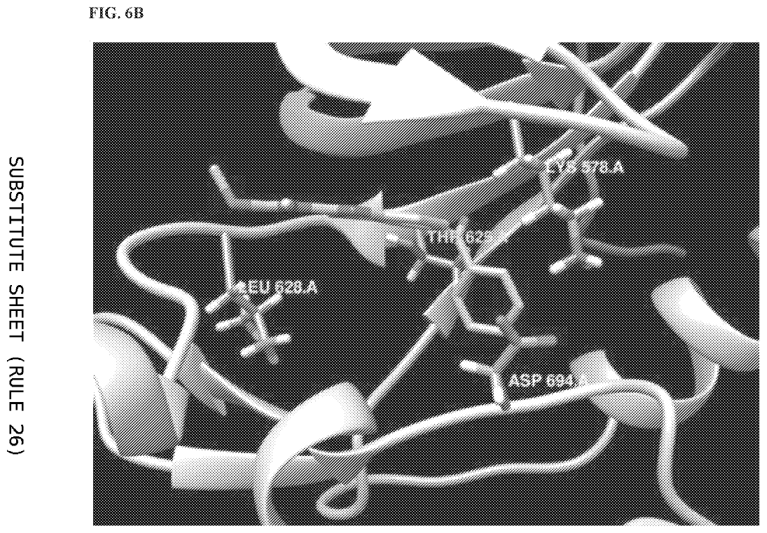

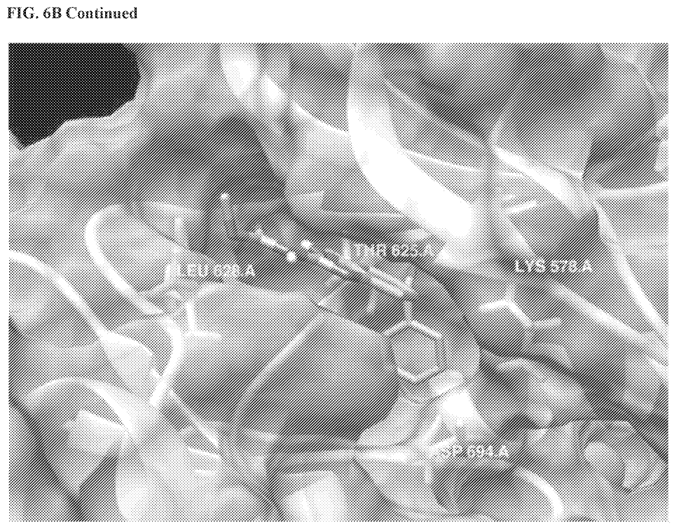

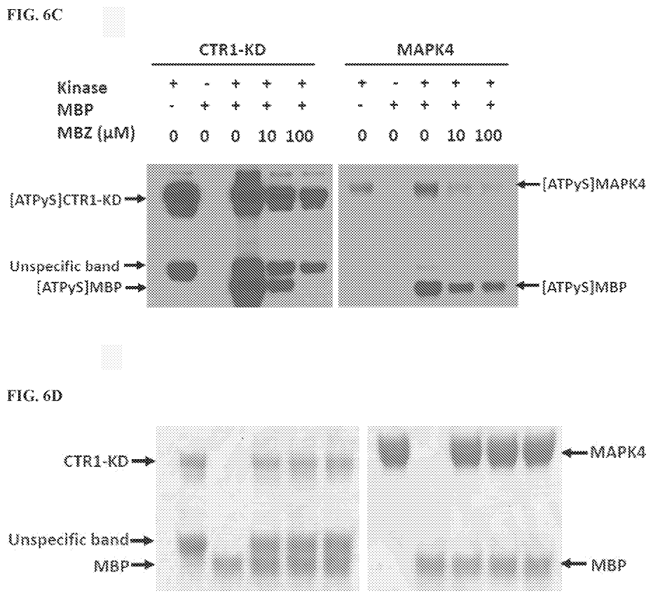

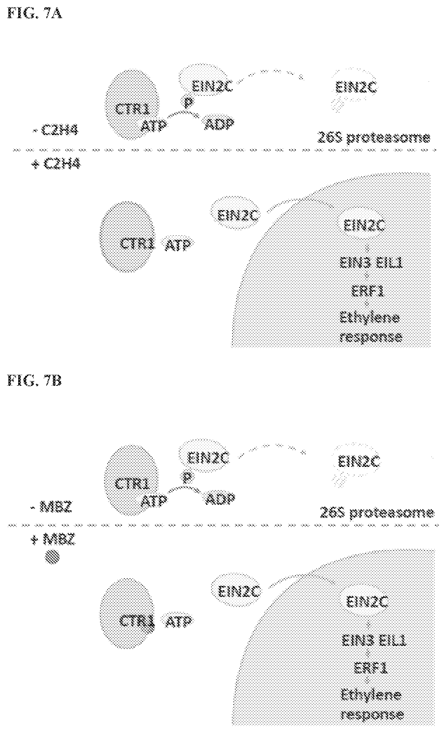

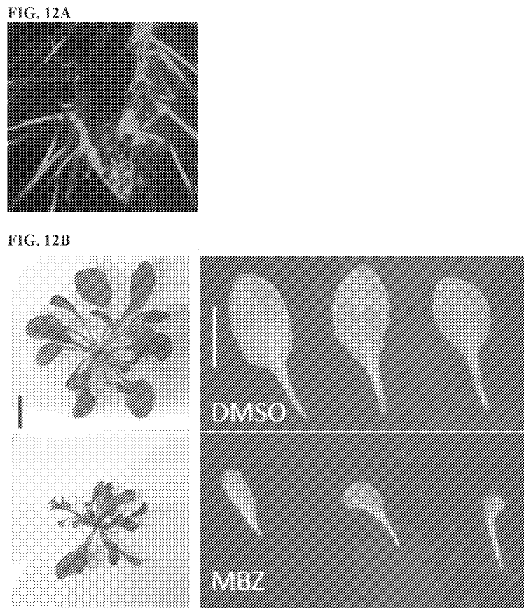

To pinpoint whether CTR1 and EIN2 is the target of MBZ, we capitalized on what is known about the activity of MBZ in mammalian systems. There, MBZ has been reported to be an inhibitor of the serine/threonine kinase Homo sapiens (hs) MAPK14 (Ariey-Bonnet et al., 2020). While EIN2 is a membrane protein with an N-terminal 12 transmembrane domain and a hydrophilic C-terminus (Alonso et al., 1999), CTR1 is a serine/threonine protein kinase (Kieber et al., 1993) that phosphorates EIN2 at the C-terminus (Ju et al., 2012; Wen et al., 2012; Qiao et al., 2012). Because both of hsMAPK14 and CTR1 are serine/threonine kinases, we hypothesized that CTR1 is the direct target of MBZ in plants. Since it was shown that there are 4 key amino acid residues of the catalytic site of hsMAPK14 that are relevant for MBZ binding (Ariey-Bonnet et al., 2020), we first tested in silico whether these residues were conserved between hsMAPK14 and CTR1. For this, we aligned the amino-acid sequence of the Arabidopsis CTR1 and hsMAPK14 kinase domains (FIG. 6A, FIG. 11A, FIG. 11B). We found 3 (Lys578, Thr 625, and Asp 694) of the 4 critical amino acid residues were conserved between the CTR1 kinase domain and that of hsMAPK14 (FIG. 6A). The different key residue is Met 109 in hsMAPK14, which has been changed to Leu 628 in CTR1. Because both Met and Leu are amino acids with hydrophobic side chain and similar structure, it appeared possible that the replacement of Met by Leu in the CTR1 kinase domain does not significantly change the binding pocket structure for MBZ. We therefore hypothesized that the kinase domains of CTR1 and MAPK14 might have similar structures and function with regards to binding MBZ (FIG. 11B).

To test this hypothesis, we first performed a molecular docking analysis using the published crystal structures of CTR1³⁹ (PDB: 3PPZ) and MBZ (CHEMBL685). We found that MBZ is predicted to bind into the pocket of the CTR1 kinase domain (FIG. 6B) formed by the aforementioned key amino acids (FIG. 6A, FIG. 6B, FIG. 11B). Overall, these analyses indicated that CTR1 could be the target of MBZ in Arabidopsis.

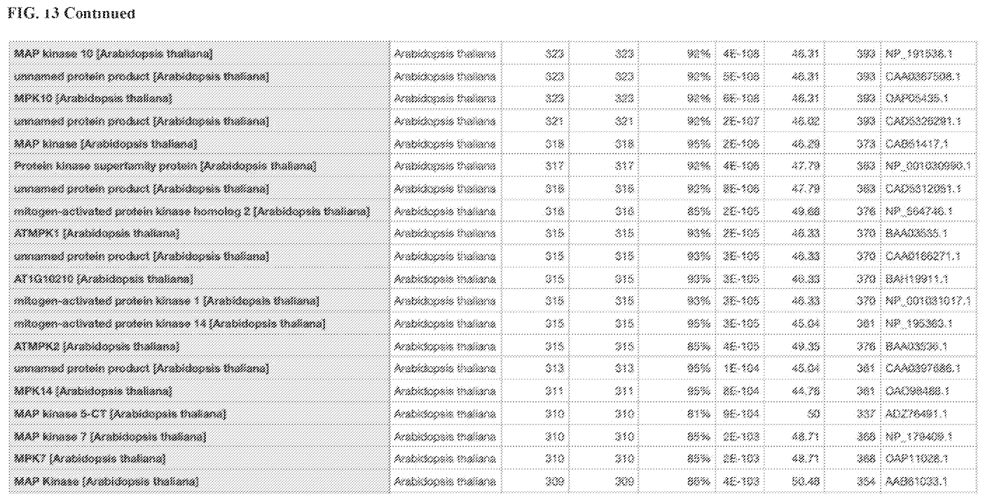

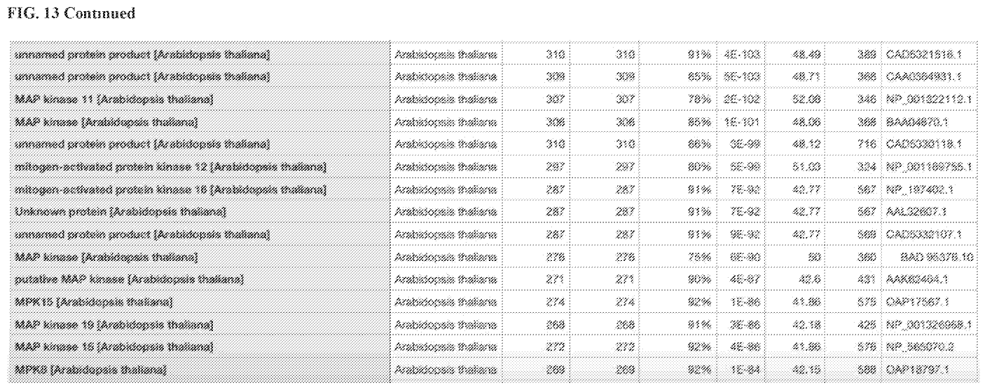

To further test this model, we expressed the recombinant kinase domains (KD) of CTR1 and MAPK4 (a MAPK protein in Arabidopsis with a highly similar amino acid sequence to hsMAPK14) (FIG. 13, FIG. 11C, FIG. 11D). We then confirmed the kinase inhibitory activity of MBZ through an in vitro phosphorylation assay via anti-Thiophosphate-Ester antibodies. MBZ inhibited the kinase activities of both CTR1-KD and MPK4 in a concentration dependent manner, but the inhibition for CTR1-KD was much more pronounced (FIG. 6C). In particular, 10 μM MBZ strongly inhibited the kinase activity of CTR1-KD, while 100 μM MBZ totally blocked its activity. This assay demonstrated that MBZ directly inhibited the kinase activity of CTR1-KD, strongly suggesting that CTR1 is a direct target of MBZ in Arabidopsis.

Taken together, our data support a model in which MBZ is a small molecular inhibitor of the CTR1 kinase activity and thereby triggers the ethylene signaling pathway by permitting EIN2C translocation into the nucleus, which in turn triggers ethylene downstream responses (FIGS. 7A-7b).

### Example 6: MBZ is a Potent Inhibitor of the Kinase Activity of CTR1

Our chemical genetic screen led to the identification of MBZ as a potent inhibitor of the kinase activity of CTR1, which is a negative modulator of the ethylene signaling pathway. MBZ therefore is an activator of ethylene signaling, and to our knowledge is the first specific small molecule that acts as a regulator of ethylene signaling.

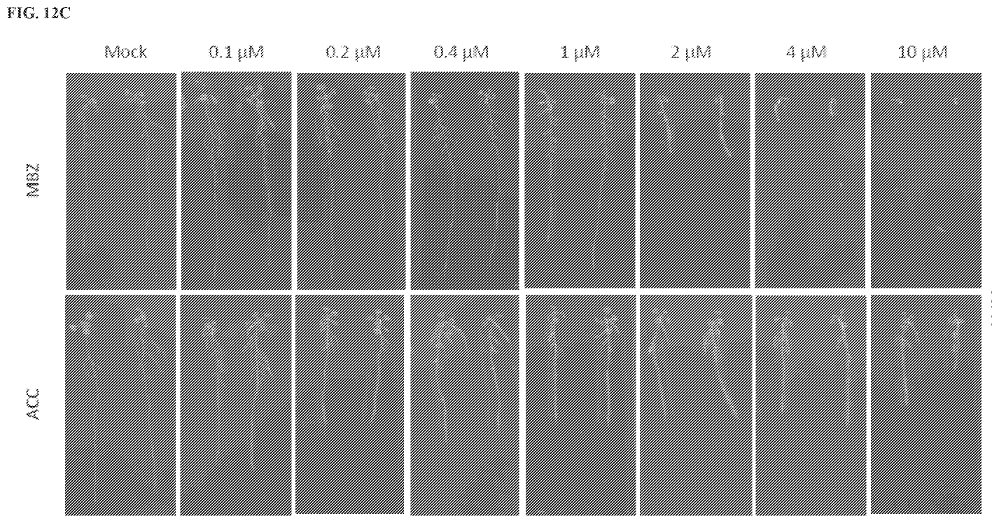

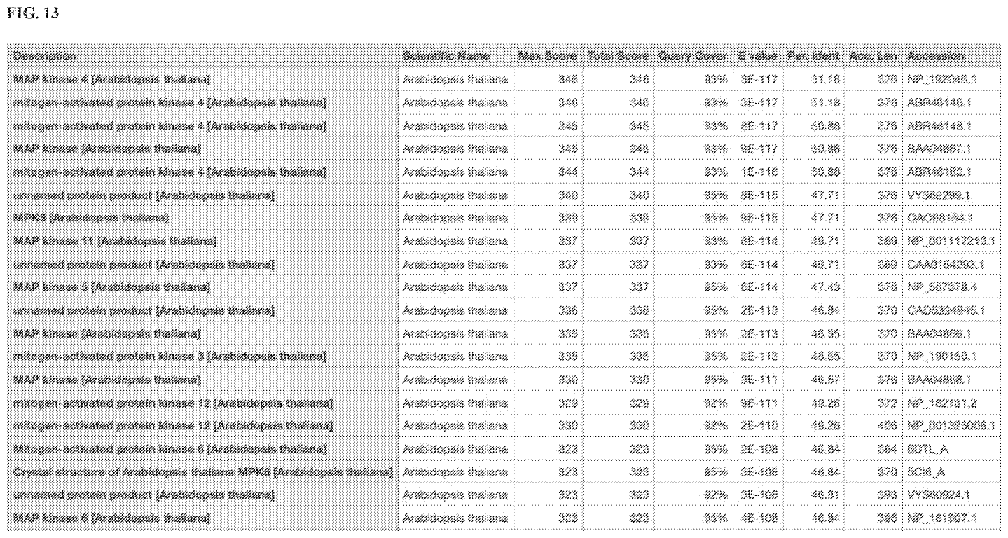

While most of the effects of MBZ are related to its activation of ethylene signaling, our RNA profiling indicated additional effects of MBZ. Consistently, treatment with high concentration of MBZ or long term treatment with it, led to strongly inhibited plant growth and development, including a disrupted root apical meristem and morphological changes of leaves. These phenotypes have not been observed upon ethylene treatment (FIG. 12A, FIG. 12B), raising the possibility that MBZ may have other targets in plants. In that case the prime candidate for being and additional target is MAPK4, which is the Arabidopsis kinase with the highest amino acid sequence similarity to the human MBZ target hsMAPK14 (FIG. 13). However, compared to CTR1, the effect of MBZ on MAPK4 kinase activity was much less pronounced (FIG. 6C). A careful sequence alignment showed that despite the high similarity, key amino acids in MAPK4 that are relevant for MBZ binding are not conserved (FIG. 11C). One explanation for the observed effects of MBZ that go beyond observed ethylene effects, might be that continuous activation of ethylene signaling via CTR1 might have more complicated or compounded effects than are elicited by external ethylene or ACC treatment (FIG. 12C). This could be due to cascading effects upon continuous stimulation of ethylene signaling or due to the transport of MBZ within the plant, which could activate ethylene signaling predominantly in tissues that are more or less permeable to MBZ compared to ACC or ethylene. Support for the additional effects of MBZ being caused by continuous ethylene signaling activation comes from the ctr1-1 mutant, which is dwarfed and shows aberration in leaf forms and thereby displays much stronger phenotypes than elicited with ACC or ethylene treatment (Kieber et al., 1993) (FIG. 5B, FIG. 5D, FIG. 12C). Future work will determine whether MBZ has additional relevant targets in Arabidopsis beyond CTR1.

As demonstrated by the Examples provided above, one of the most intriguing effects of MBZ on the plant is its potent ability to change GSA. This suggests that ethylene signaling is involved in the regulation of the gravitropic setpoint angle in lateral roots. Consistently, we found that at certain concentrations, ACC treatment had a weak but distinct effect on root angle and displayed concentration-dependent effects when applying a large range of concentrations (FIG. 12C). However, even at higher concentrations, ACC treatment could not entirely mimic the effects of MBZ treatment in increasing lateral root angle. Since MBZ acts directly on ethylene signaling and ACC needs to be converted into ethylene in plant, it could be expected a number of downstream processes are different upon ACC and MBZ treatments. These include transport, cell-type specific permeability of ACC and MBZ, as well as cell-type specific conversion rates of ACC to ethylene. Moreover, ACC was recently found to be a signaling molecule to regulate pollen tube attraction, which is independent of ethylene pathway (Mou, 2020). Overall, ACC and MBZ are therefore expected to give rise to different dosage-dependent, and distinct differences in direct and indirect responses. However, strong direct evidence for the impact of ethylene signaling in regulating lateral root setpoint angle is provided by the RSA phenotype of the ctr1-1 mutant. In ctr1-1, ethylene signaling is constitutively activated. Much like upon MBZ treatment, an increased lateral root angle phenotype is observed in the ctr1-1 mutant (FIG. 5D, FIG. 5E). One reason why ethylene has so far not been implicated as a major player in the regulation of lateral root angles might be that it reduces lateral root formation by itself (Negi et al., 2008) making the study of lateral root angles more difficult. Our study strongly suggests that it will be worthwhile to explore the role of ethylene in root gravitropism and RSA. Since ethylene signaling is a widely conserved process in land plants, exploring the ability of MBZ or genetic perturbation of the ethylene pathway to change RSA in crop species might be highly worthwhile for RSA engineering.

### Example 7: Application of MBZ for Regulating Lateral Root Angles and Root System Architecture in Tobacco

The small molecule Mebendazole (MBZ) disclosed herewith can be applied to tobacco or other plant species in order to activate ethylene signalling and modulate lateral root angles and enhance the capacity for root systems to efficiently update water and nutrients, thereby improving tobacco plant survival and productivity.

The effects of MBZ application to tobacco plants, tobacco plant parts, or tobacco plant tissue cultures in a dry or liquid form are being assessed with regards to increased lateral root angle or gravitropic set-point angle. The small molecule, MBZ, can be used as an ethylene signalling activator to sustain and improve productivity in tobacco despite environmental changes and/or stresses.

### Example 8: Application of MBZ for Regulating Lateral Root Angles and Root System Architecture in Rice

The small molecule Mebendazole (MBZ) disclosed herewith can be applied to rice or other plant species in order to activate ethylene signalling and modulate lateral root angles and enhance the capacity for root systems to efficiently update water and nutrients, thereby improving rice plant survival and productivity.

The effects of MBZ application to rice plants, rice plant parts, or rice plant tissue cultures in a dry or liquid form are being assessed with regards to increased lateral root angle or gravitropic set-point angle. The small molecule, MBZ, can be used as an ethylene signalling activator to sustain and improve productivity in rice despite environmental changes and/or stresses.

### Example 9: Application of MBZ for Regulating Lateral Root Angles and Root System Architecture in Corn

The small molecule Mebendazole (MBZ) disclosed herewith can be applied to corn or other plant species in order to activate ethylene signalling and modulate lateral root angles and enhance the capacity for root systems to efficiently update water and nutrients, thereby improving corn plant survival and productivity.

The effects of MBZ application to corn plants, corn plant parts, or corn plant tissue cultures in a dry or liquid form are being assessed with regards to increased lateral root angle or gravitropic set-point angle. The small molecule, MBZ, can be used as an ethylene signalling activator to sustain and improve productivity in corn despite environmental changes and/or stresses.

### Example 10: Application of MBZ for Regulating Lateral Root Angles and Root System Architecture in Soybean

The small molecule Mebendazole (MBZ) disclosed herewith can be applied to soybean or other plant species in order to activate ethylene signalling and modulate lateral root angles and enhance the capacity for root systems to efficiently update water and nutrients, thereby improving soybean plant survival and productivity.

The effects of MBZ application to soybean plants, soybean plant parts, or soybean plant tissue cultures in a dry or liquid form are being assessed with regards to increased lateral root angle or gravitropic set-point angle. The small molecule, MBZ, can be used as an ethylene signalling activator to sustain and improve productivity in soybean despite environmental changes and/or stresses.

### Example 11: Application of MBZ for Regulating Lateral Root Angles and Root System Architecture in Wheat

The small molecule Mebendazole (MBZ) disclosed herewith can be applied to wheat or other plant species in order to activate ethylene signalling and modulate lateral root angles and enhance the capacity for root systems to efficiently update water and nutrients, thereby improving wheat plant survival and productivity.

The effects of MBZ application to wheat plants, wheat plant parts, or wheat plant tissue cultures in a dry or liquid form are being assessed with regards to increased lateral root angle or gravitropic set-point angle. The small molecule, MBZ, can be used as an ethylene signalling activator to sustain and improve productivity in wheat despite environmental changes and/or stresses.

### Example 12: Application of MBZ for Regulating Lateral Root Angles and Root System Architecture in Cotton

The small molecule Mebendazole (MBZ) disclosed herewith can be applied to cotton or other plant species in order to activate ethylene signalling and modulate lateral root angles and enhance the capacity for root systems to efficiently update water and nutrients, thereby improving cotton plant survival and productivity.

The effects of MBZ application to cotton plants, cotton plant parts, or cotton plant tissue cultures in a dry or liquid form are being assessed with regards to increased lateral root angle or gravitropic set-point angle. The small molecule, MBZ, can be used as an ethylene signalling activator to sustain and improve productivity in cotton despite environmental changes and/or stresses.

### Example 13: Application of MBZ for Regulating Lateral Root Angles and Root System Architecture in Canola

The small molecule Mebendazole (MBZ) disclosed herewith can be applied to canola or other plant species in order to activate ethylene signalling and modulate lateral root angles and enhance the capacity for root systems to efficiently update water and nutrients, thereby improving canola plant survival and productivity.

The effects of MBZ application to canola plants, canola plant parts, or canola plant tissue cultures in a dry or liquid form are being assessed with regards to increased lateral root angle or gravitropic set-point angle. The small molecule, MBZ, can be used as an ethylene signalling activator to sustain and improve productivity in canola despite environmental changes and/or stresses.

### Example 14: Application of MBZ for Regulating Lateral Root Angles and Root System Architecture in Barley

The small molecule Mebendazole (MBZ) disclosed herewith can be applied to barley or other plant species in order to activate ethylene signalling and modulate lateral root angles and enhance the capacity for root systems to efficiently update water and nutrients, thereby improving barley plant survival and productivity.

The effects of MBZ application to barley plants, barley plant parts, or barley plant tissue cultures in a dry or liquid form are being assessed with regards to increased lateral root angle or gravitropic set-point angle. The small molecule, MBZ, can be used as an ethylene signalling activator to sustain and improve productivity in barley despite environmental changes and/or stresses.

### Example 15: Application of MBZ for Regulating Lateral Root Angles and Root System Architecture in Sorghum

The small molecule Mebendazole (MBZ) disclosed herewith can be applied to sorghum or other plant species in order to activate ethylene signalling and modulate lateral root angles and enhance the capacity for root systems to efficiently update water and nutrients, thereby improving sorghum plant survival and productivity.

The effects of MBZ application to sorghum plants, sorghum plant parts, or sorghum plant tissue cultures in a dry or liquid form are being assessed with regards to increased lateral root angle or gravitropic set-point angle. The small molecule, MBZ, can be used as an ethylene signalling activator to sustain and improve productivity in sorghum despite environmental changes and/or stresses.

### Example 16: Application of MBZ for Regulating Lateral Root Angles and Root System Architecture in Radish

The small molecule Mebendazole (MBZ) disclosed herewith can be applied to radish or other plant species in order to activate ethylene signalling and modulate lateral root angles and enhance the capacity for root systems to efficiently update water and nutrients, thereby improving radish plant survival and productivity.

The effects of MBZ application to radish plants, radish plant parts, or radish plant tissue cultures in a dry or liquid form are being assessed with regards to increased lateral root angle or gravitropic set-point angle. The small molecule, MBZ, can be used as an ethylene signalling activator to sustain and improve productivity in radish despite environmental changes and/or stresses.

### Example 17: Application of MBZ for Regulating Lateral Root Angles and Root System Architecture in Crimson Clover

The small molecule Mebendazole (MBZ) disclosed herewith can be applied to Crimson Clover or other plant species in order to activate ethylene signalling and modulate lateral root angles and enhance the capacity for root systems to efficiently update water and nutrients, thereby improving Crimson Clover plant survival and productivity.

The effects of MBZ application to Crimson Clover plants, Crimson Clover plant parts, or Crimson Clover plant tissue cultures in a dry or liquid form are being assessed with regards to increased lateral root angle or gravitropic set-point angle. The small molecule, MBZ, can be used as an ethylene signalling activator to sustain and improve productivity in Crimson Clover despite environmental changes and/or stresses.

### Example 18: Application of MBZ for Regulating Lateral Root Angles and Root System Architecture in Field Pennycress/CoverCress

The small molecule Mebendazole (MBZ) disclosed herewith can be applied to Field Pennycress/CoverCress or other plant species in order to activate ethylene signalling and modulate lateral root angles and enhance the capacity for root systems to efficiently update water and nutrients, thereby improving Field Pennycress/CoverCress plant survival and productivity.

The effects of MBZ application to Field Pennycress/CoverCress plants, Field Pennycress/CoverCress plant parts, or Field Pennycress/CoverCress plant tissue cultures in a dry or liquid form are being assessed with regards to increased lateral root angle or gravitropic set-point angle. The small molecule, MBZ, can be used as an ethylene signalling activator to sustain and improve productivity in Field Pennycress/CoverCress despite environmental changes and/or stresses.

### Example 19: Application of MBZ for Regulating Lateral Root Angles and Root System Architecture in Annual Ryegrass

The small molecule Mebendazole (MBZ) disclosed herewith can be applied to Annual Ryegrass or other plant species in order to activate ethylene signalling and modulate lateral root angles and enhance the capacity for root systems to efficiently update water and nutrients, thereby improving Annual Ryegrass plant survival and productivity.

The effects of MBZ application to Annual Ryegrass plants, Annual Ryegrass plant parts, or Annual Ryegrass plant tissue cultures in a dry or liquid form are being assessed with regards to increased lateral root angle or gravitropic set-point angle. The small molecule, MBZ, can be used as an ethylene signalling activator to sustain and improve productivity in Annual Ryegrass despite environmental changes and/or stresses.

Table 1 presents sequence information disclosed herewith.

TABLE 1
Sequence Information

| Name | Gene Model | NCBI Accession (UniProtKB) | SEQ ID NO |
|---|---|---|---|
| ERF1 | AT3G23240 | NP_188965.1 (Q8LDC8) | SEQ ID NO: 1 |
| ETR1 | AT1G66340 | NP_176808.3 (P49333) | SEQ ID NO: 2 |
| CTR1 | AT5g03730 | NP_195993.1 (Q05609) | SEQ ID NO: 3 |
| EIN2 | AT5G03280 | NP_195948.1 (Q9S814) | SEQ ID NO: 4 |
| EIN3 | AT3g20770 | NP_188713.1 (O24606) | SEQ ID NO: 5 |
| EIL1 | AT2G27050 | NP_180273.1 (Q9SLH0) | SEQ ID NO: 6 |
| hsMAPK14 | HGNC:6876 | AAH31574.1 (Q16539) | SEQ ID NO: 7 |
| MAPK4 | AT4G01370 | NP_192046.1 (Q39024) | SEQ ID NO: 8 |
| MAPK6 | AT2G43790 | NP_181907.1 (Q39026) | SEQ ID NO: 9 |
| MAPK9 | AT3G18040 | NP_566595.1 (Q9LV37) | SEQ ID NO: 10 |
| MAPK11 | ATIG01560 | NP_001117210.1 (Q9LMM5) | SEQ ID NO: 11 |
| MAPK12 | AT2G46070 | NP_182131.2 (Q8GYQ5) | SEQ ID NO: 12 |

### FURTHER NUMBERED EMBODIMENTS OF THE DISCLOSURE

Other subject matter contemplated by the present disclosure is set out in the following numbered embodiments:
1. A method of modulating an ethylene signaling pathway in a plant or plant tissue culture comprising administering a small molecule that acts as a regulator of ethylene signaling.
2. The method of embodiment 1 wherein the small molecule has a molecular weight less than 300 g/mol.
3. The method of embodiment 1, wherein the small molecule inhibits or blocks the kinase activity of CTR1.
4. The method of embodiment 1, wherein the small molecule is a positive regulator of ethylene signaling.
5. The method of embodiment 1, wherein the small molecule inhibits a negative regulator of ethylene signaling.
6. The method of embodiment 1, wherein the small molecule modulates gravitropic set-point angle (GSA) of the plant or plant tissue culture.
7. The method of embodiment 6, wherein the GSA is significantly increased.
8. The method of embodiment 1, wherein the modulating leads to changes in root system architecture of the plant or plant tissue culture relative to a check plant or plant tissue culture, respectively, that is not administered the small molecule.
9. The method of embodiment 1, wherein the modulating leads to changes in lateral root angle of the plant or plant tissue culture when compared to a check plant or plant tissue culture, respectively, that is not administered the small molecule.
10. The method of embodiment 9, wherein the changes in lateral root angle cause the lateral roots of the plant or plant tissue culture to grow in a more horizontal direction when compared to a check plant or plant tissue culture, respectively, that is not administered the small molecule.
11. The method of embodiment 1, wherein the administering is accomplished by adding the small molecule to a growing medium used to grow the plant or the plant tissue culture.
12. The method of embodiment 1, wherein the administering is accomplished by applying the small molecule to the growing medium used to grow the plant or the plant tissue culture.
13. The method of embodiment 12, wherein the applying is accomplished by spraying the small molecule onto the plant or plant tissue culture.
14. The method of embodiment 12, wherein the applying is accomplished by using a liquid comprising the small molecule.
15. The method of embodiment 1, wherein the small molecule is an anthelmintic agent.
16. The method of embodiment 1, wherein the small molecule is a synthetic benzimidazole derivate.
17. The method of embodiment 1, wherein the small molecule is a benzimidazole anthelmintic agent.
18. The method of embodiment 1, wherein the small molecule is mebendazole.
19. A method of activating an ethylene signaling pathway in a plant or a plant tissue culture comprising the steps of administering to a plant or a plant tissue culture a small molecule that targets CTR1 protein that is a negative modulator of the ethylene signalling pathway.
20. The method of embodiment 19, wherein said small molecule inhibits a kinase activity of CTR1.
21. The method of embodiment 19, wherein said small molecule binds to a pocket of the CTR1 kinase domain.
22. The method of embodiment 21, wherein the CTR1 kinase domain is present in SEQ ID NO: 3.
23. The method of embodiment 19, wherein the small molecule is mebendazole.
24. The method of embodiment 19, wherein the small molecule modulates gravitropic set-point angle (GSA) of the plant or plant tissue culture.
25. The method of embodiment 24, wherein the GSA is significantly increased.
26. The method of embodiment 19, wherein the administering of said small molecule leads to changes in root system architecture of the plant or plant tissue culture relative to a check plant or plant tissue culture, respectively, that is not administered the small molecule.
27. The method of embodiment 19, wherein the administering of said small molecule leads to changes in lateral root angle of the plant or plant tissue culture when compared to a check plant or plant tissue culture, respectively, that is not administered the small molecule.
28. The method of embodiment 27, wherein the changes in lateral root angle cause the lateral roots of the plant or plant tissue culture to grow in a more horizontal direction when compared to a check plant or plant tissue culture, respectively, that is not administered the small molecule.
29. The method of embodiment 19, wherein the administering is accomplished by adding the small molecule to a growing medium used to grow the plant or the plant tissue culture.
30. The method of embodiment 19, wherein the administering is accomplished by applying the small molecule to the growing medium used to grow the plant or the plant tissue culture.
31. The method of embodiment 30, wherein the applying is accomplished by spraying the small molecule onto the plant or plant tissue culture.
32. The method of embodiment 30, wherein the applying is accomplished by using a liquid comprising the small molecule.

### INCORPORATION BY REFERENCE

All references, articles, publications, patents, patent publications, and patent applications cited herein within the above text and/or cited below are incorporated by reference in their entireties for all purposes. However, mention of any reference, article, publication, patent, patent publication, and patent application cited herein is not, and should not be taken as acknowledgment or any form of suggestion that they constitute valid prior art or form part of the common general knowledge in any country in the world.

### REFERENCES

1 Lynch, J. P. Harnessing root architecture to address global challenges. Plant J, doi:10.1111/tpj.15560 (2021).
2 Lynch, J. P. Root phenotypes for improved nutrient capture: an underexploited opportunity for global agriculture. New Phytol 223, 548-564, doi:10.1111/nph.15738 (2019).
3 Poirier, V., Roumet, C. & Munson, A. D. The root of the matter: Linking root traits and soil organic matter stabilization processes. Soil Biology and Biochemistry 120, 246-259, (2018).
4 Uga, Y. et al. Control of root system architecture by DEEPER ROOTING 1 increases rice yield under drought conditions. Nat Genet 45, 1097-1102, doi:10.1038/ng.2725 (2013).
Roychoudhry, S., Del Bianco, M., Kieffer, M. & Kepinski, S. Auxin controls gravitropic setpoint angle in higher plant lateral branches. Curr Biol 23, 1497-1504, doi:10.1016/j.cub.2013.06.034 (2013).
6 Rosquete, M. R. et al. An auxin transport mechanism restricts positive orthogravitropism in lateral roots. Curr Biol 23, 817-822, doi:10.1016/j.cub.2013.03.064 (2013).
7 Xiao, G. & Zhang, Y. Adaptive Growth: Shaping Auxin-Mediated Root System Architecture. Trends Plant Sci 25, 121-123, doi:10.1016/j.tplants.2019.12.001 (2020).
8 Waidmann, S. et al. Cytokinin functions as an asymmetric and anti-gravitropic signal in lateral roots. Nat Commun 10, 3540, doi:10.1038/s41467-019-11483-4 (2019).
9 Ju, C. et al. Conservation of ethylene as a plant hormone over 450 million years of evolution. Nat Plants 1, 14004, doi:10.1038/nplants.2014.4 (2015).
10 S F Yang, a. & Hoffman, N. E. Ethylene Biosynthesis and its Regulation in Higher Plants. Annual Review of Plant Physiology 35, 155-189, doi:10.1146/annurev.pp. 35.060184.001103 (1984).
11 Pattyn, J., Vaughan-Hirsch, J. & Van de Poel, B. The regulation of ethylene biosynthesis: a complex multilevel control circuitry. New Phytol 229, 770-782, doi:10.1111/nph.16873 (2021).
12 Guzman, P. & Ecker, J. R. Exploiting the triple response of Arabidopsis to identify ethylene-related mutants. Plant Cell 2, 513-523, doi:10.1 105/tpc.2.6.513 (1990).
13 Guo, H. & Ecker, J. R. The ethylene signaling pathway: new insights. Curr Opin Plant Biol 7, 40-49, doi:10.1016/j.pbi.2003.11.011 (2004).
14 Chang, C., Kwok, S. F., Bleecker, A. B. & Meyerowitz, E. M. Arabidopsis ethylene-response gene ETR1: similarity of product to two-component regulators. Science 262, 539-544, doi:10.1126/science.8211181 (1993).
15 Sakai, H. et al. ETR2 is an ETR1-like gene involved in ethylene signaling in Arabidopsis. Proc Natl Acad Sci USA 95, 5812-5817, doi:10.1073/pnas.95.10.5812 (1998).
16 Hua, J. et al. EIN4 and ERS2 are members of the putative ethylene receptor gene family in Arabidopsis. Plant Cell 10, 1321-1332, doi:10.1105/tpc.10.8.1321 (1998).
17 Kieber, J. J., Rothenberg, M., Roman, G., Feldmann, K. A. & Ecker, J. R. CTR1, a negative regulator of the ethylene response pathway in Arabidopsis, encodes a member of the raf family of protein kinases. Cell 72, 427-441, doi:10.1016/0092-8674(93)90119-b (1993).
18 Alonso, J. M., Hirayama, T., Roman, G., Nourizadeh, S. & Ecker, J. R. EIN2, a bifunctional transducer of ethylene and stress responses in Arabidopsis. Science 284, 2148-2152, doi:10.1126/science.284.5423.2148 (1999).
19 Ju, C. et al. CTR1 phosphorylates the central regulator EIN2 to control ethylene hormone signaling from the ER membrane to the nucleus in Arabidopsis. Proc Natl Acad Sci USA 109, 19486-19491, doi:10.1073/pnas.1214848109 (2012).
20 Wen, X. et al. Activation of ethylene signaling is mediated by nuclear translocation of the cleaved EIN2 carboxyl terminus. Cell Res 22, 1613-1616, doi:10.1038/cr.2012.145 (2012).
21 Qiao, H. et al. Processing and subcellular trafficking of ER-tethered EIN2 control response to ethylene gas. Science 338, 390-393, doi:10.1126/science.1225974 (2012).
22 Chao, Q. et al. Activation of the ethylene gas response pathway in Arabidopsis by the nuclear protein ETHYLENE-INSENSITIVE3 and related proteins. Cell89, 1133-1144, doi:10.1016/s0092-8674(00)80300-1 (1997).
23 An, F. et al. Ethylene-induced stabilization of ETHYLENE INSENSITIVE3 and EIN3-LIKE1 is mediated by proteasomal degradation of EIN3 binding F-box 1 and 2 that requires EIN2 in Arabidopsis. Plant Cell 22, 2384-2401, doi:10.1105/tpc.110.076588 (2010).
24 Muller, M. & Munne-Bosch, S. Ethylene Response Factors: A Key Regulatory Hub in Hormone and Stress Signaling. Plant Physiol 169, 32-41, doi:10.1104/pp. 15.00677 (2015).
25 Vaseva, I I et al. The plant hormone ethylene restricts Arabidopsis growth via the epidermis. Proc Natl Acad Sci USA 115, E4130-E4139, doi:10.1073/pnas.1717649115 (2018).
26 Le, J., Vandenbussche, F., Van Der Straeten, D. & Verbelen, J. P. In the early response of Arabidopsis roots to ethylene, cell elongation is up- and down-regulated and uncoupled from differentiation. Plant Physiol 125, 519-522, doi:10.1104/pp. 125.2.519 (2001).
27 Feng, Y. et al. Ethylene promotes root hair growth through coordinated EIN3/EIL1 and RHD6/RSL1 activity in Arabidopsis. Proc Natl Acad Sci USA 114, 13834-13839, doi:10.1073/pnas.1711723115 (2017).
28 Negi, S., Ivanchenko, M. G. & Muday, G. K. Ethylene regulates lateral root formation and auxin transport in Arabidopsis thaliana. Plant J 55, 175-187, doi:10.1111/j.1365-313X.2008.03495.x (2008).
29 He, W. et al. A small-molecule screen identifies L-kynurenine as a competitive inhibitor of TAA1/TAR activity in ethylene-directed auxin biosynthesis and root growth in Arabidopsis. Plant Cell 23, 3944-3960, doi:10.1105/tpc.111.089029 (2011).
30 Sun, X. et al. Pyrazinamide and derivatives block ethylene biosynthesis by inhibiting ACC oxidase. Nat Commun 8, 15758, doi:10.1038/ncomms15758 (2017).
31 Zhu, Y. et al. A phenotype-directed chemical screen identifies ponalrestat as an inhibitor of the plant flavin monooxygenase YUCCA in auxin biosynthesis. J Biol Chem 294, 19923-19933, doi:10.1074/jbc.RA119.010480 (2019).
32 Ristova, D., Giovannetti, M., Metesch, K. & Busch, W. Natural genetic variation shapes root system responses to phytohormones in Arabidopsis. Plant J 96, 468-481, doi:10.1111/tpj.14034 (2018).
33 Ogura, T. et al. Root System Depth in Arabidopsis Is Shaped by EXOCYST70A3 via the Dynamic Modulation of Auxin Transport. Cell 178, 400-412 e416, doi:10.1016/j.cell.2019.06.021 (2019).
34 Liao, C. Y. et al. Reporters for sensitive and quantitative measurement of auxin response. Nat Methods 12, 207-210, 202 p following 210, doi:10.1038/nmeth.3279 (2015).
35 Chen, X. et al. Inhibition of cell expansion by rapid ABP1-mediated auxin effect on microtubules. Nature 516, 90-93, doi:10.1038/nature13889 (2014).
36 Chang, K. N. et al. Temporal transcriptional response to ethylene gas drives growth hormone cross-regulation in Arabidopsis. Elife 2, e00675, doi:10.7554/eLife.00675 (2013).
37 De Cnodder, T., Vissenberg, K., Van Der Straeten, D. & Verbelen, J. P. Regulation of cell length in the Arabidopsis thaliana root by the ethylene precursor 1-aminocyclopropane-1-carboxylic acid: a matter of apoplastic reactions. New Phytol 168, 541-550, doi:10.1111/j.1469-8137.2005.01540.x (2005).
38 Ariey-Bonnet, J. et al. In silico molecular target prediction unveils mebendazole as a potent MAPK14 inhibitor. Mol Oncol 14, 3083-3099, doi:10.1002/1878-0261.12810 (2020).
39 Mayerhofer, H., Panneerselvam, S. & Mueller-Dieckmann, J. Protein kinase domain of CTR1 from Arabidopsis thaliana promotes ethylene receptor cross talk. J Mol Biol 415, 768-779, doi:10.1016/j.jmb.2011.11.046 (2012).
40 Mou, W. et al. Ethylene-independent signaling by the ethylene precursor ACC in Arabidopsis ovular pollen tube attraction. Nat Commun 11, 4082, doi:10.1038/s41467-020-17819-9 (2020).
41 Nakamura, M., Naoi, K., Shoji, T. & Hashimoto, T. Low concentrations of propyzamide and oryzalin alter microtubule dynamics in Arabidopsis epidermal cells. Plant Cell Physiol 45, 1330-1334, doi:10.1093/pcp/pch300 (2004).
42 Li, B. et al. GSNOR provides plant tolerance to iron toxicity via preventing iron-dependent nitrosative and oxidative cytotoxicity. Nat Commun 10, 3896, doi:10.1038/s41467-019-11892-5 (2019).
43 Berardini, T. Z. et al. The Arabidopsis information resource: Making and mining the “gold standard” annotated reference plant genome. Genesis 53, 474-485, doi:10.1002/dvg.22877 (2015).
44 Dobin, A. et al. STAR: ultrafast universal RNA-seq aligner. Bioinformatics 29, 15-21, doi:10.1093/bioinformatics/bts635 (2013).
45 Robinson, M. D., McCarthy, D. J. & Smyth, G. K. edgeR: a Bioconductor package for differential expression analysis of digital gene expression data. Bioinformatics 26, 139-140, doi:10.1093/bioinformatics/btp616 (2010).
46 Leinonen, R., Sugawara, H., Shumway, M. & International Nucleotide Sequence Database, C. The sequence read archive. Nucleic Acids Res 39, D19-21, doi:10.1093/nar/gkq1019 (2011).
47 Mayerhofer, H., Mueller-Dieckmann, C. & Mueller-Dieckmann, J. Cloning, expression, purification and preliminary X-ray analysis of the protein kinase domain of constitutive triple response 1 (CTR1) from Arabidopsis thaliana. Acta Crystallogr Sect F Struct Biol Cryst Commun 67, 117-120, doi:10.1107/S1744309110047640 (2011).
48 Allen, J. J. et al. A semisynthetic epitope for kinase substrates. Nat Methods 4, 511-516, doi:10.1038/nmeth1048 (2007).
49 Ogura, T., Goeschl, C. & Busch, W. A Multiplexed, Time-Resolved Assay of Root Gravitropic Bending on Agar Plates. Methods Mol Biol 2368, 61-70, doi:10.1007/978-1-0716-1677-2_4 (2022).

1. A method of modulating an ethylene signaling pathway in a plant or plant tissue culture comprising administering a small molecule that acts as a regulator of ethylene signaling.

2. The method of claim 1, wherein the small molecule has a molecular weight less than 300 g/mol.

3. The method of claim 1, wherein the small molecule inhibits or blocks the kinase activity of Constitutive Triple Response1 (CTR1).

4. The method of claim 1, wherein the small molecule is a positive regulator of ethylene signaling.

5. The method of claim 1, wherein the small molecule inhibits a negative regulator of ethylene signaling.

6. The method of claim 1, wherein the small molecule modulates gravitropic set-point angle (GSA) of the plant or plant tissue culture, and the GSA is significantly increased.

7. (canceled)

8. The method of claim 1, wherein the modulating leads to changes in root system architecture of the plant or plant tissue culture relative to a check plant or plant tissue culture, respectively, that is not administered the small molecule.

9. The method of claim 1, wherein the modulating leads to changes in lateral root angle of the plant or plant tissue culture when compared to a check plant or plant tissue culture, respectively, that is not administered the small molecule.

10. The method of claim 9, wherein the changes in lateral root angle cause the lateral roots of the plant or plant tissue culture to grow in a more horizontal direction when compared to a check plant or plant tissue culture, respectively, that is not administered the small molecule.

11.-14. (canceled)

15. The method of claim 1, wherein the small molecule is
an anthelmintic agent,
a synthetic benzimidazole derivate, or
a benzimidazole anthelmintic agent.

16.-17. (canceled)

18. The method of claim 1, wherein the small molecule is mebendazole.

19. A method of activating an ethylene signaling pathway in a plant or a plant tissue culture comprising administering to a plant or a plant tissue culture a small molecule that targets CTR1 protein that is a negative modulator of the ethylene signaling pathway.

20. The method of claim 19, wherein said small molecule inhibits a kinase activity of CTR1.

21. The method of claim 19, wherein said small molecule binds to a pocket of the CTR1 kinase domain.

22. The method of claim 21, wherein the CTR1 kinase domain is present in SEQ ID NO: 3.

23. The method of claim 19, wherein the small molecule is mebendazole.

24. The method of claim 19, wherein the small molecule modulates gravitropic set-point angle (GSA) of the plant or plant tissue culture, and the GSA is significantly increased.

25. (canceled)

26. The method of claim 19, wherein the administering of said small molecule leads to changes in root system architecture of the plant or plant tissue culture relative to a check plant or plant tissue culture, respectively, that is not administered the small molecule.

27. The method of claim 19, wherein the administering of said small molecule leads to changes in lateral root angle of the plant or plant tissue culture when compared to a check plant or plant tissue culture, respectively, that is not administered the small molecule.

28. The method of claim 27, wherein the changes in lateral root angle cause the lateral roots of the plant or plant tissue culture to grow in a more horizontal direction when compared to a check plant or plant tissue culture, respectively, that is not administered the small molecule.

29.-32. (canceled)


SEQUENCE LISTING (12 sequences)

SEQ ID NO: 1 - protein, 218 aa, Arabidopsis thaliana
MDPFLIQSPFSGFSPEYSIGSSPDSFSSSSSNNYSLPFNENDSEEMFLYGLIEQSTQQTYIDSDSQDLPIKSVSSRKSEKSYRGVRRRPWGKFAAEIRDSTRNGIRVWLGTFESAEEAALAYDQAAFSMRGSSAILNFSAERVQESLSEIKYTYEDGCSPVVALKRKHSMRRRMTNKKTKDSDFDHRSVKLDNVVVFEDLGEQYLEELLGSSENSGTW

SEQ ID NO: 2 - protein, 738 aa, Arabidopsis thaliana
MEVCNCIEPQWPADELLMKYQYISDFFIAIAYFSIPLELIYFVKKSAVFPYRWVLVQFGAFIVLCGATHLINLWTFTTHSRTVALVMTTAKVLTAVVSCATALMLVHIIPDLLSVKTRELFLKNKAAELDREMGLIRTQEETGRHVRMLTHEIRSTLDRHTILKTTLVELGRTLALEECALWMPTRTGLELQLSYTLRHQHPVEYTVPIQLPVINQVFGTSRAVKISPNSPVARLRPVSGKYMLGEVVAVRVPLLHLSNFQINDWPELSTKRYALMVLMLPSDSARQWHVHELELVEVVADQVAVALSHAAILEESMRARDLLMEQNVALDLARREAETAIRARNDFLAVMNHEMRTPMHAIIALSSLLQETELTPEQRLMVETILKSSNLLATLMNDVLDLSRLEDGSLQLELGTFNLHTLFREVLNLIKPIAVVKKLPITLNLAPDLPEFVVGDEKRLMQIILNIVGNAVKFSKQGSISVTALVTKSDTRAADFFVVPTGSHFYLRVKVKDSGAGINPQDIPKIFTKFAQTQSLATRSSGGSGLGLAISKRFVNLMEGNIWIESDGLGKGCTAIFDVKLGISERSNESKQSGIPKVPAIPRHSNFTGLKVLVMDENGVSRMVTKGLLVHLGCEVTTVSSNEECLRVVSHEHKVVFMDVCMPGVENYQIALRIHEKFTKQRHQRPLLVALSGNTDKSTKEKCMSFGLDGVLLKPVSLDNIRDVLSDLLEPRVLYEGM

SEQ ID NO: 3 - protein, 821 aa, Arabidopsis thaliana
MEMPGRRSNYTLLSQFSDDQVSVSVTGAPPPHYDSLSSENRSNHNSGNTGKAKAERGGFDWDPSGGGGGDHRLNNQPNRVGNNMYASSLGLQRQSSGSSFGESSLSGDYYMPTLSAAANEIESVGFPQDDGFRLGFGGGGGDLRIQMAADSAGGSSSGKSWAQQTEESYQLQLALALRLSSEATCADDPNFLDPVPDESALRTSPSSAETVSHRFWVNGCLSYYDKVPDGFYMMNGLDPYIWTLCIDLHESGRIPSIESLRAVDSGVDSSLEAIIVDRRSDPAFKELHNRVHDISCSCITTKEVVDQLAKLICNRMGGPVIMGEDELVPMWKECIDGLKEIFKVVVPIGSLSVGLCRHRALLFKVLADIIDLPCRIAKGCKYCNRDDAASCLVRFGLDREYLVDLVGKPGHLWEPDSLLNGPSSISISSPLRFPRPKPVEPAVDFRLLAKQYFSDSQSLNLVFDPASDDMGFSMFHRQYDNPGGENDALAENGGGSLPPSANMPPQNMMRASNQIEAAPMNAPPISQPVPNRANRELGLDGDDMDIPWCDLNIKEKIGAGSFGTVHRAEWHGSDVAVKILMEQDFHAERVNEFLREVAIMKRLRHPNIVLFMGAVTQPPNLSIVTEYLSRGSLYRLLHKSGAREQLDERRRLSMAYDVAKGMNYLHNRNPPIVHRDLKSPNLLVDKKYTVKVCDFGLSRLKASTFLSSKSAAGTPEWMAPEVLRDEPSNEKSDVYSFGVILWELATLQQPWGNLNPAQVVAAVGFKCKRLEIPRNLNPQVAAIIEGCWTNEPWKRPSFATIMDLLRPLIKSAVPPPNRSDL

SEQ ID NO: 4 - protein, 1294 aa, Arabidopsis thaliana
MEAEIVNVRPQLGFIQRMVPALLPVLLVSVGYIDPGKWVANIEGGARFGYDLVAITLLFNFAAILCQYVAARISVVTGKHLAQICNEEYDKWTCMFLGIQAEFSAILLDLTMVVGVAHALNLLFGVELSTGVFLAAMDAFLFPVFASFLENGMANTVSIYSAGLVLLLYVSGVLLSQSEIPLSMNGVLTRLNGESAFALMGLLGASIVPHNFYIHSYFAGESTSSSDVDKSSLCQDHLFAIFGVFSGLSLVNYVLMNAAANVFHSTGLVVLTFHDALSLMEQVFMSPLIPVVFLMLLFFSSQITALAWAFGGEVVLHDFLKIEIPAWLHRATIRILAVAPALYCVWTSGADGIYQLLIFTQVLVAMMLPCSVIPLFRIASSRQIMGVHKIPQVGEFLALTTFLGFLGLNVVFVVEMVFGSSDWAGGLRWNTVMGTSIQYTTLLVSSCASLCLILWLAATPLKSASNRAEAQIWNMDAQNALSYPSVQEEEIERTETRRNEDESIVRLESRVKDQLDTTSVTSSVYDLPENILMTDQEIRSSPPEERELDVKYSTSQVSSLKEDSDVKEQSVLQSTVVNEVSDKDLIVETKMAKIEPMSPVEKIVSMENNSKFIEKDVEGVSWETEEATKAAPTSNFTVGSDGPPSFRSLSGEGGSGTGSLSRLQGLGRAARRHLSAILDEFWGHLYDFHGQLVAEARAKKLDQLFGTDQKSASSMKADSFGKDISSGYCMSPTAKGMDSQMTSSLYDSLKQQRTPGSIDSLYGLQRGSSPSPLVNRMQMLGAYGNTTNNNNAYELSERRYSSLRAPSSSEGWEHQQPATVHGYQMKSYVDNLAKERLEALQSRGEIPTSRSMALGTLSYTQQLALALKQKSQNGLTPGPAPGFENFAGSRSISRQSERSYYGVPSSGNTDTVGAAVANEKKYSSMPDISGLSMSARNMHLPNNKSGYWDPSSGGGGYGASYGRLSNESSLYSNLGSRVGVPSTYDDISQSRGGYRDAYSLPQSATTGTGSLWSRQPFEQFGVAERNGAVGEELRNRSNPINIDNNASSNVDAEAKLLQSFRHCILKLIKLEGSEWLFGQSDGVDEELIDRVAAREKFIYEAEAREINQVGHMGEPLISSVPNCGDGCVWRADLIVSFGVWCIHRVLDLSLMESRPELWGKYTYVLNRLQGVIDPAFSKLRTPMTPCFCLQIPASHQRASPTSANGMLPPAAKPAKGKCTTAVTLLDLIKDVEMAISCRKGRTGTAAGDVAFPKGKENLASVLKRYKRRLSNKPVGMNQDGPGSRKNVTAYGSLG

SEQ ID NO: 5 - protein, 628 aa, Arabidopsis thaliana
MMFNEMGMCGNMDFFSSGSLGEVDFCPVPQAEPDSIVEDDYTDDEIDVDELERRMWRDKMRLKRLKEQDKGKEGVDAAKQRQSQEQARRKKMSRAQDGILKYMLKMMEVCKAQGFVYGIIPENGKPVTGASDNLREWWKDKVRFDRNGPAAITKYQAENNIPGIHEGNNPIGPTPHTLQELQDTTLGSLLSALMQHCDPPQRRFPLEKGVPPPWWPNGKEDWWPQLGLPKDQGPAPYKKPHDLKKAWKVGVLTAVIKHMFPDIAKIRKLVRQSKCLQDKMTAKESATWLAIINQEESLARELYPESCPPLSLSGGSCSLLMNDCSQYDVEGFEKESHYEVEELKPEKVMNSSNFGMVAKMHDFPVKEEVPAGNSEFMRKRKPNRDLNTIMDRTVFTCENLGCAHSEISRGFLDRNSRDNHQLACPHRDSRLPYGAAPSRFHVNEVKPVVGFPQPRPVNSVAQPIDLTGIVPEDGQKMISELMSMYDRNVQSNQTSMVMENQSVSLLQPTVHNHQEHLQFPGNMVEGSFFEDLNIPNRANNNNSSNNQTFFQGNNNNNNVFKFDTADHNNFEAAHNNNNNSSGNRFQLVFDSTPFDMASFDYRDDMSMPGVVGTMDGMQQKQQDVSIWF

SEQ ID NO: 6 - protein, 584 aa, Arabidopsis thaliana
MMMFNEMGMYGNMDFFSSSTSLDVCPLPQAEQEPVVEDVDYTDDEMDVDELEKRMWRDKMRLKRLKEQQSKCKEGVDGSKQRQSQEQARRKKMSRAQDGILKYMLKMMEVCKAQGFVYGIIPEKGKPVTGASDNLREWWKDKVRFDRNGPAAIAKYQSENNISGGSNDCNSLVGPTPHTLQELQDTTLGSLLSALMQHCDPPQRRFPLEKGVSPPWWPNGNEEWWPQLGLPNEQGPPPYKKPHDLKKAWKVGVLTAVIKHMSPDIAKIRKLVRQSKCLQDKMTAKESATWLAIINQEEVVARELYPESCPPLSSSSSLGSGSLLINDCSEYDVEGFEKEQHGFDVEERKPEIVMMHPLASFGVAKMQHFPIKEEVATTVNLEFTRKRKQNNDMNVMVMDRSAGYTCENGQCPHSKMNLGFQDRSSRDNHQMVCPYRDNRLAYGASKFHMGGMKLVVPQQPVQPIDLSGVGVPENGQKMITELMAMYDRNVQSNQTPPTLMENQSMVIDAKAAQNQQLNFNSGNQMFMQQGTNNGVNNRFQMVFDSTPFDMAAFDYRDDWQTGAMEGMGKQQQQQQQQQDVSIWF

SEQ ID NO: 7 - protein, 360 aa, Homo sapiens
MSQERPTFYRQELNKTIWEVPERYQNLSPVGSGAYGSVCAAFDTKTGLRVAVKKLSRPFQSIIHAKRTYRELRLLKHMKHENVIGLLDVFTPARSLEEFNDVYLVTHLMGADLNNIVKCQKLTDDHVQFLIYQILRGLKYIHSADIIHRDLKPSNLAVNEDCELKILDFGLARHTDDEMTGYVATRWYRAPEIMLNWMHYNQTVDIWSVGCIMAELLTGRTLFPGTDHIDQLKLILRLVGTPGAELLKKISSESARNYIQSLTQMPKMNFANVFIGANPLAVDLLEKMLVLDSDKRITAAQALAHAYFAQYHDPDDEPVADPYDQSFESRDLLIDEWKSLTYDEVISFVPPPLDQEEMES

SEQ ID NO: 8 - protein, 376 aa, Arabidopsis thaliana
MSAESCFGSSGDQSSSKGVATHGGSYVQYNVYGNLFEVSRKYVPPLRPIGRGAYGIVCAATNSETGEEVAIKKIGNAFDNIIDAKRTLREIKLLKHMDHENVIAVKDIIKPPQRENFNDVYIVYELMDTDLHQIIRSNQPLTDDHCRFFLYQLLRGLKYVHSANVLHRDLKPSNLLLNANCDLKLGDFGLARTKSETDFMTEYVVTRWYRAPELLLNCSEYTAAIDIWSVGCILGETMTREPLFPGKDYVHQLRLITELIGSPDDSSLGFLRSDNARRYVRQLPQYPRQNFAARFPNMSAGAVDLLEKMLVFDPSRRITVDEALCHPYLAPLHDINEEPVCVRPFNFDFEQPTLTEENIKELIYRETVKFNPQDSV

SEQ ID NO: 9 - protein, 395 aa, Arabidopsis thaliana
MDGGSGQPAADTEMTEAPGGFPAAAPSPQMPGIENIPATLSHGGRFIQYNIFGNIFEVTAKYKPPIMPIGKGAYGIVCSAMNSETNESVAIKKIANAFDNKIDAKRTLREIKLLRHMDHENIVAIRDIIPPPLRNAFNDVYIAYELMDTDLHQIIRSNQALSEEHCQYFLYQILRGLKYIHSANVLHRDLKPSNLLLNANCDLKICDFGLARVTSESDFMTEYVVTRWYRAPELLLNSSDYTAAIDVWSVGCIFMELMDRKPLFPGRDHVHQLRLLMELIGTPSEEELEFLNENAKRYIRQLPPYPRQSITDKFPTVHPLAIDLIEKMLTFDPRRRITVLDALAHPYLNSLHDISDEPECTIPFNFDFENHALSEEQMKELIYREALAFNPEYQQ

SEQ ID NO: 10 - protein, 510 aa, Arabidopsis thaliana
MDPHKKVALETEFFTEYGEASRYQIQEVIGKGSYGVVASAIDTHSGEKVAIKKINDVFEHVSDATRILREIKLLRLLRHPDIVEIKHVMLPPSRREFRDIYVVFELMESDLHQVIKANDDLTPEHYQFFLYQLLRGLKFIHTANVFHRDLKPKNILANSDCKLKICDFGLARVSFNDAPSAIFWTDYVATRWYRAPELCGSFFSKYTPAIDIWSIGCIFAEMLTGKPLFPGKNVVHQLDIMTDLLGTPPPEAIARIRNEKARRYLGNMRRKPPVPFTHKFPHVDPLALRLLHRLLAFDPKDRPSAEEALADPYFYGLANVDREPSTQPIPKLEFEFERRKITKEDVRELIYREILEYHPQMLQEYLRGGEQTSFMYPSGVDRFKRQFAHLEENYGKGEKGSPLQRQHASLPRERVPAPKKENGSHNHDIENRSIASLVTTLESPPTSQHEGSDYRNGTSQTGYSARSLLKSASISASKCIGMKPRNKSEYGESNNDTVDALSQKVAALHT

SEQ ID NO: 11 - protein, 369 aa, Arabidopsis thaliana
MSIEKPFFGDDSNRGVSINGGRYVQYNVYGNLFEVSKKYVPPLRPIGRGASGIVCAAWNSETGEEVAIKKIGNAFGNIIDAKRTLREIKLLKHMDHDNVIAIIDIIRPPQPDNFNDVHIVYELMDTDLHHIIRSNQPLTDDHSRFFLYQLLRGLKYVHSANVLHRDLKPSNLLLNANCDLKIGDFGLARTKSETDFMTEYVVTRWYRAPELLLNCSEYTAAIDIWSVGCILGEIMTREPLFPGRDYVQQLRLITELIGSPDDSSLGFLRSDNARRYVRQLPQYPRQNFAARFPNMSVNAVDLLQKMLVFDPNRRITVDEALCHPYLAPLHEYNEEPVCVRPFHFDFEQPSLTEENIKELIYRESVKFNP

SEQ ID NO: 12 - protein, 372 aa, Arabidopsis thaliana
MSGESSSGSTEHCIKVVPTHGGRYVQYNVYGQLFEVSRKYVPPIRPIGRGACGIVCAAVNSVTGEKVAIKKIGNAFDNIIDAKRTLREIKLLRHMDHENVITIKDIVRPPQRDIFNDVYIVYELMDTDLQRILRSNQTLTSDQCRFLVYQLLRGLKYVHSANILHRDLRPSNVLLNSKNELKIGDFGLARTTSDTDFMTEYVVTRWYRAPELLLNCSEYTAAIDIWSVGCILGEIMTGQPLFPGKDYVHQLRLITELVGSPDNSSLGFLRSDNARRYVRQLPRYPKQQFAARFPKMPTTAIDLLERMLVFDPNRRISVDEALGHAYLSPHHDVAKEPVCSTPFSFDFEHPSCTEEHIKELIYKESVKFNPDH